SEQUENCE LISTING (92 sequences)

SEQ ID NO: 1 - DNA, 1908 bp, Lycopersicon esculentum
atggaagtctgcaattgtattgaaccgcaatggccagcggatgaattgttaatgaaataccaatacatctccgatttcttcattgcgattgcgtatttttcgattcctcttgagttgatttactttgtgaagaaatcagccgtgtttccgtatagatgggtacttgttcagtttggtgcttttatcgttctttatggagcaactcatcttattaacttatggactttcactacgcattcgagaaccgtggcgcttgtgatgactaccgcgaaggtgttgacagctgccgtgtcctgtatcacagctttgatgcttgttcacattattcctgatttgctaagtgttaaaacgcgagagttgttcttgaaaactcgagctgaagagcttgacaaggaaatgggcctaataataagacaagaagaaactggcagacatgtcaggatgctgactcatgagataagaagcacactcgacagacacacaatcttgaagactactcttgtggagctaggtaggaccttagacctggcagaatgtgctttgtggatgccatgccaaggaggcctgactttgcaactttcccataatttaaacaatctaatacctctgggatctactgtgccaattaatcttcctattatcaatgaaatttttagtagccctgaagcaatacaaattccacatacaaatcctttggcaaggatgaggaatactgttggtagatatattccaccagaagtagttgctgttcgtgtaccgcttttacacctctcaaattttactaatgactgggctgaactgtctactagaagttatgcggttatggttctggttctcccgatgaatggcttaagaaagtggcgtgaacatgagttagaacttgtgcaagttgtcgcagatcaggttgctgtcgctctttcacatgctgcaattttagaagattccatgcgagcccatgatcagctcatggaacagaatattgctttggatgtagctcgacaagaagcagagatggccatccgtgcacgtaacgacttccttgctgtgatgaaccatgaaatgagaacgcccatgcatgcagttattgctctgtgctctctgcttttagaaacagacttaactccagagcagagagttatgattgagaccatattgaagagcagcaatcttcttgcaacactgataaatgatgttctagatctttctagacttgaagatggtattcttgaactagaaaacggaacattcaatcttcatggcatcttaagagaggccgttaatttgataaagccaattgcatctttgaagaaattatctataactcttgctttggctctggatttacctattcttgctgtgggtgatgcaaaacgtcttatccaaactctcttaaacgtggcgggaaatgctgtgaagttcactaaagaaggacatatttcaattgaggcttcagttgccaaaccagagtatgcgagagattgtcatcctcctgaaatgttccctatgccaagtgatggccagttttatttgcgtgtccaggttagagatactgggtgtggaattagcccacaagatataccactagtattcaccaaatttgcagagtcacggcctacgtcaaatcgaagtactggaggggaaggtctagggcttgccatttgcagacgatttattcaacttatgaaaggtaacatttggattgagagtgagggccctggaaagggaaccactgtcacgtttgtagtgaaactcggaatctgtcaccatccaaatgcattacctctgctacctatgcctcccagaggcagattgaacaaaggtagcgatgatctcttcaggtatagacagttccgtggagatgatggtgggatgtctgtgaatgctcaacgctatcaaagaagtatgtag

SEQ ID NO: 2 - DNA, 402 bp, Oryza sativa
atggggatggcacagtcgtcttcgtcttcctcgcgcccctccgactccgagcagctagaggagcccagcaagccggtcatggcgctcgacaaggccaaggagatcgtcgcctcctcccccatcgtcgtcttcagcaagacttattgccctttctgcgcccgagtgaagcgattgctggcagagctggcagcaagttacaaggctgttgaattggatgtggaaagtgatgggtctgagctgcagtcagctcttgccgattggactggacagagaactgttccttgtgtcttcattaaagggaaacatattggtggctgtgacgataccatggcgatgcacaaaggagggaacttggtccctctgctgacggaggcaggagcaatcgccactccttccctgtag

SEQ ID NO: 3 - DNA, 717 bp, Oryza sativa
atgggcgaggaggcgccggaggagtacgagctgggcggcggggaggacgagcgggtgatggagtgggagacggggctgcccggcgccgacgagctgaccccgctgtcgcagccgctggtgccggcggggctggcggcggcgttccgcatcccgccggagcccgggcgcacgctgctcgacgtgcaccgcgcgtcggcggcgacggtgtcccggttgcggcgcgcgtcgtcgtcgtcgtcgagctcgttcccggcgttcgcgtcgaagggagcgggaacgggagcggacgaggcggagtcagggggaggcgcggatggggggaacgggaacaccaacaacagcagcagcaagagggcgcggctggtgtggacgccgcagctgcacaagaggttcgtggaggtggtggcgcacctggggatgaagaacgcggtgcccaagacgatcatgcagctgatgaacgtggagggcctcacccgggagaacgtcgccagccacctccagaagtatcgcctctacgtgaagcggatgcagggcctctccaacgagggcccttccccctccgaccacatcttcgcctccacccccgtcccccacgcctccctccacgaccaggttccttctccttaccacccccacccccaccaccactcctacaacaacgccgcctatgccgccaccgtctcctcctaccaccactaccaccacgccaaccactag

SEQ ID NO: 4 - DNA, 759 bp, Capsicum annuum
atgaatcaagatatggccttagaacagcttgacactacctttaacaaacacgatactccattagggaaatggaagtcaatgaacgatgaagttgaagagaatatttctggtggcttcgactgtaacatatgcctggattgtgtgcacgaacctgtgataactttatgcggtcatctttactgctggccttgcatttacaaatggatttatttccagagtgtttcttcagaaaattcggatcagcaacaaccgcaatgccctgtttgcaaggctgaagtctcagaaaaaaccttgattccactctatggacgcggtggtcaatctacaaaaccatccgaaggaaaggctccgaatcttggcatagtgatcccacaaaggccccctagtccaaggtgtggtggtcacttcttgttaccaactactgattcaaatccatcccagctacttcaacgacgaggttatcaacagcagtctcaaacacgtcaaccggcttatcagggtagctacatgtcttcgcccatgctcagccctggtggtgcgactgcgaatatgttacaacactccatgattggagaagtagcctatgcaagaatttttggcaactcatcaacaactatgtatacatatccaaactcttataatctagcaatcagcagtagcccaagaatgagaaggcaattatcacaggctgatagatcacttggcagaatatgttttttcctattttgttgctttgtcacatgtctaatcttgttttag

SEQ ID NO: 5 - DNA, 942 bp, Arabidopsis thaliana
atggacgagggagttatagcagtttccgccatggatgctttcgagaagcttgagaaagttggtgaagggacatacgggaaagtttacagagccagagagaaagctaccgggaaaatcgtcgctctaaagaagacgcgtctccatgaggacgaagaaggcgttccttccaccactctccgcgagatctccattttgcgaatgctcgctcgtgatcctcacgtcgtcaggttaatggatgttaagcaaggactaagcaaagaaggcaaaactgtactgtacctggtttttgaatacatggacactgatgtcaagaaattcatcagaagtttccgtagcactggcaagaacattccaacccaaactatcaagagcttgatgtatcaactatgcaaaggtatggcattctgccatggtcacgggatattgcacagagatctcaagcctcacaatctcttgatggatcccaagacaatgaggctcaaaatagcagatcttggtttagccagagccttcactctgccaatgaagaagtatacccatgagatattaactctttggtatagagctccagaggttcttcttggtgccacccattactctacagctgtggatatgtggtctgttggctgcatatttgctgaacttgtgaccaaccaagcaatctttcagggagactctgagctccaacagctcctccatattttcaagttgtttgggacacccaatgaagaaatgtggccaggagtgagcacactcaagaactggcatgaatacccacagtggaaaccatcgactctatcctctgctgttccaaacctcgacgaggctggagttgatcttctatctaaaatgctgcagtacgagccagcgaaacgaatctcagcaaagatggctatggagcatccttactttgatgatctgccagaaaagtcctctctctag

SEQ ID NO: 6 - DNA, 2958 bp, Arabidopsis thaliana
atgttcctatgcttttgcccttgccacgtacctatcatgagtcgcctttctccggccaccggaatctcttcccgcctccgcttctccattggtttgagttccgatggacgattaattcccttcggattccggtttcggagaaacgatgtcccgtttaaacgccgcttgagatttgtgatcagagcgcagctctctgaagctttttctcccgatttaggtttggattctcaggctgtgaaatcccgcgatacatcaaacttgccttggattggtccagttccaggggacattgctgaggttgaggcgtattgtagaatttttagatcagctgagcgactacatgtagcgttgatggagacactatgcaaccctgtgactggtgaatgtcgagtaccgtatgatttctcaccggaggaaaaaccattgttggaggacaaaatagtatcagtgcttggttgtatattatctcttttaaacaaaggaaggaaagaaattctctctgggaggtcatcttctatgaattcatttaatttggatgatgttggggttgcagaggagtcgcttccaccacttgctgttttcaggggtgaaatgaaacggtgttgtgaaagcttacacattgctcttgagaattatctgacgccggatgatgagagaagtggaattgtttggaggaaattacagaagcttaaaaatgtctgctacgacgctggttttccacgcagtgataactatccttgtcaaacactttttgcgaattgggaccctatttactcgtcaaatacgaaagaggatattgattcctacgagtctgagattgcattttggaggggaggacaggtaacccaagaaggattgaagtggttgatagaaaacggatttaaaacaattgttgacctgagagctgaaatcgttaaggatacattctaccagacggcacttgatgatgcaatttcccttgggaaaattacagtggtgcaaattccaattgatgtcaggatggctcctaaagctgagcaggtcgagctgtttgcttctattgtatcagatagcagcaaaagaccgatatatgttcacagtaaagaaggtgtttggagaacttctgcgatggtttctaggtggaagcagtacatgacacgaccgatcacgaaagaaattccagtttcagaagagtcaaagcgtcgggaggtttctgaaactaagcttggatcaaatgctgtagtatctggtaagggtgtacctgatgagcagactgataaagtctctgaaatcaatgaggttgatagtagatctgcttcaagccagagcaaggaatctggaaggtttgagggagatacatctgcatcagaatttaatatggtgagcgatcctcttaaatctcaagttccaccaggcaatattttttcaagaaaagaaatgtctaaattcctgaagagcaaaagtattgctcctgctggttatcttactaatccgtccaaaatattgggaacagtgccaactccacaattttcatatactggtgtgacaaacggaaatcagattgttgataaagattcgataagaagacttgcggagacaggaaactccaatgggacccttctacctacaagttctcaaagtttagattttggcaacgggaagttttcaaatggaaatgtgcatgcgtctgataacaccaacaaaagtatatcggacaacaggggaaatggcttctctgcagcccctattgctgtgcctcctagtgataacttaagtcgcgctgtaggatcccattcggttcgagagtctcagactcagagaaataatagtggttcctcctcggattccagtgatgatgaagctggagctattgagggaaatatgtgtgcttctgccactggtgtagtaagggtgcagtcgagaaagaaagcagagatgttcttagtccgaactgatggagtgtcttgtacaagggaaaaggtgacagaatcctctctggccttcacacatccaagtactcaacagcagatgcttctttggaaaactaccccaaaaactgtcttactgctgaagaagctcgggcaagaactgatggaggaagctaaagaggctgcctctttcttgtatcaccaagagaatatgaatgttctggttgaacctgaggtgcatgatgtatttgccaggattccagggtttggctttgtccagaccttctacattcaggacacgagcgatctccatgaaagggttgattttgtggcatgcttagggggggatggggtgatattacatgcatcaaacttgttcaaaggagccgtccctcccgttgtttcatttaatctggggtctcttggatttctcacttcacatccatttgaggacttcaggcaagacctcaaacgagtcatccatgggaataacacgctagatggggtttatataactcttcgaatgcgtcttcgttgcgaaatctatcgtaaaggcaaagcaatgcctggtaaagtgtttgatgttctgaacgagattgttgttgatcgaggatccaacccatacctttctaagatcgaatgttatgagcacgaccgtcttatcacgaaggtacaaggcgatggagttatagtagccactcctacaggaagtactgcttattctacagcagcaggaggttccatggtgcatccaaacgttccttgcatgctgttcactccaatctgcccacattccctgtcgttcagaccagttatacttccagattctgcaaaactcgagttaaagattccagatgatgctcgaagcaatgcatgggtttcgtttgatggaaagagaagacaacaactttcaaggggagattcggtgagaatatacatgagccaacatccactcccaactgtcaacaaatctgatcaaaccggtgattggtttagaagcttaatccgttgcttaaactggaacgagcgtcttgatcaaaaggctctctag

SEQ ID NO: 7 - DNA, 1137 bp, Oryza sativa
atggcgaccgccgttgcgtcccaggttgctgtctctgctccggctggctcggatcgcggcttgaggagttctgggatccagggtagcaacaatattagctttagcaacaaatcatgggttggcaccacattggcgtgggagagcaaggccacgcgaccgaggcatgcgaacaaggtgctctgcatgtcagttcagcaagcgagcgaaagcaaggttgctgtcaagcctcttgatttggagagtgctaacgagccgccgctcaacacatacaaaccaaaggagccttacaccgccacaattgtctcggttgagaggatcgtaggccccaaggctccaggagagacatgccacattgttattgatcatggtggcaatgtgccttactgggaggggcaaagctatggcattattcctccaggggagaacccgaagaagcctggtgcaccacataatgtccgtctttattcaattgcatctacaaggtatggagattcattcgatggaaggaccactagtttatgtgtgcgccgtgccgtttattatgatcctgaaactggcaaggaggacccctcaaaaaatggtgtctgcagtaacttcctatgtaattcaaaaccaggggacaaggttaaagtgacaggtccgtcaggcaaaataatgctcctgcctgaggaagatccaaatgcaactcacatcatgatagctactggcactggtgttgctccattccgtggctacctacgccgtatgttcatggaagatgtcccaaagtacagatttggtggcttggcctggctcttccttggtgtggctaacactgacagccttctctatgatgaagagttcacaagctaccttaagcagtatccagacaatttcaggtatgacaaagcgctaagcagggagcagaaaaacaagaacgctggcaagatgtatgtccaggacaagatcgaggagtacagcgacgagatcttcaagctcttggatggcggcgcgcacatctacttctgtggtttgaaggggatgatgcctgggattcaagacaccctcaagaaagtggcggagcagagaggggagagctgggagcagaagctatcccagctcaagaagaacaagcaatggcacgttgaggtctactag

SEQ ID NO: 8 - DNA, 1680 bp, Cyanidium caldarium
atgtgcggcatcctagccgtgctaggctctagcctgcccgttgaggagctgagggaactcgttaagtcctgcactaagaagctctaccatcgcggccctgacgaggagcaatacttcatcagcgaggacggctggtgcggcctcggcttcgcccgcctcaagatcgtggaccctgagcacggcgtgcagcccatgttcaacgaccagcgcaccgtgtggtccgtgaccaacggcgaactctacaaccacgaggaaatccgcaagaccgagcttaacaacatgaccctccactcccactccgactgcgagatcatgatcccgctctacgagaagtatgtgtcttcccagcgctacgaccacgacatccagtatgtgtacaacctcctgcgcggcgtgttcgcctcctgcctcgtggacctcaaacgcggcttcttcatggctggccgcgaccctatcggcgtgagggccctcttctacggcacctccaaggacggagccgtgtggttcgcctccgaggccaaggccatcgtggacgtatgcgactacgtgaccgccttcatacccggcaccttcgtgaagggctaccgtggaagggaacaagccttctccttcactcgttactacgagcctgtgtactggcacgaccactggatgcctgtgtctcctgtggactaccagctcctgcacgacaccttcgtcctctcctgcaagaggcgtctcatgagcgacgtccctatcggtgtcttcatcagcggtgggcttggttcgtctctggtcgccagcgtcgccaagaggcttcttgaccctaactacgacttccacagcttcgcctgcggtctggagggtgctccggacgtcgccgcagcgcagagggtcgctgacttccttgggaccaagcaccatgtcctgactttcactgtcgaggagggtatccaggctctggaccaagtcatctaccacctggagacttacgacgtcactactgtcagggcgagcacgccgatgtacctattgtctggtctttgcaagaaatacgtcaaggtcgttctgtctggtgaaggtgctgacgaaatcttcggtggatacctttacttccacaacgctccgaacgagatcgctttccaccaggaggtcgttcgtagagtcaaactgctctacacggctgatgtccttcgtggagatagagctacggcagcgcagtctcttgagcttcgtgttccgttcctggatagagatttccttgatgttgcgatgtcgattcatccgcgtgagaaggttacgtcgaaacacagaattgagaagtacattattcggtatgcgtttagtaaggagttctgtggcgaggagtatttgccagatgacatcctttggcggcagaaggaacagtttagtgatggtgttggttacagttggattgatgggttgaaggcgtattgtgagaaggcggtttcggatgcggatttgcagaatgcggcacaacggtttccacatgatacaccaacaacaaaggaagcgtacgtctatcgggcgatcttcgagaaacacttcggaaattgtaaagcagttcaaggtttacgggaatcagttgcacgatgggttccaatgtggtcagattcaacagatccatcaggacgagcacagaaggtccatgtagcagcatactcgaatggaggagattag

SEQ ID NO: 9 - DNA, 1551 bp, Arabidopsis thaliana
atgggattgggcggcgatcaatcctttgttcccgtcatggattccggccaagtccgcctcaaggagctcggctacaagcaagagctcaagcgcgatctctcggtcttctccaatttcgccatctccttctccatcatatcggtgctcactggtatcaccaccacctacaacaccggcttaagattcggcggcactgtcactctggtctacggatggttcctggccggctccttcacaatgtgcgttgggttatctatggccgagatctgctcctcttaccctacctccggtggtctctactactggagtgctatgctcgctggccctcgttgggctcctcttgcctcttggatgactggctggttcaacatcgttggtcagtgggcagtgacggccagcgttgacttctctctggcacagttgattcaggtgatcgtccttctctccaccggcggtagaaacggcggcggttataaaggatcagactttgttgtgattggtatccatggtgggatcctcttcatccacgctcttctcaacagcctccccatctccgtcttgtctttcattggacagcttgctgctctttggaatctcctcggggttttggtgctcatgattctgattcctttggtttctacggaaagagcaaccactaagtttgtctttaccaatttcaacactgataatggccttggcatcaccagctacgcttacatattcgttttgggactcctcatgagccagtacaccattacagggtatgatgcctctgcccacatgacagaagagacagtcgacgcagacaagaacgggcccagaggaataatcagtgcaattggtatatcaattctgtttggatggggttatatattgggcataagctatgccgtcacagacataccttctcttctgagtgagaccaacaactctggtgggtatgccattgctgagatcttctacttagctttcaagaataggtttgggagcggtactggtggaatcgtgtgcttaggcgttgttgcggttgctgtgtttttctgtggcatgagctctgtcaccagcaattccaggatggcgtatgcgttttcgagagatggagcgatgccaatgtcgccgttatggcacaaagtgaacagcagagaggtccccattaatgcggtttggctctctgctctcatatcattttgtatggccctcacctcactggggagcatagtggcgttccaagcaatggtgtcgatcgcaacgattggactgtacatagcatacgcaatcccgattatattgagagtgacgcttgcgcgcaacaccttcgtacctggaccattcagcctgggaaaatacggaatggtagtcgggtgggtggcagtcctgtgggttgtaaccatatcagtcctcttctccttacccgtggcatatcccataacagcagagacactcaactacactccggtggcagttgctggtttggtggccataaccctctcatattggcttttcagtgcccgccactggtttacgggtcccatctccaacattcttagctag

SEQ ID NO: 10 - DNA, 1362 bp, Arabidopsis thaliana
atggcttctttaatgttatctctcggttccacttctctgttaccgcgcgagattaacaaggataagctaaagcttggaacttctgcttcgaacccgttcctaaaagcaaagtcttttagcagagtgactatgacggttgcagtgaagccttctcgtttcgagggtataactatggctccaccagaccctattcttggagtcagtgaagcattcaaagctgacactaacgggatgaaactcaatcttggtgttggtgcttatcgtactgaggaactccagccttatgtgcttaatgttgttaaaaaggcggagaatttgatgttggagagaggagataacaaagagtatcttccaattgaggggttggcagcattcaacaaggctactgctgagttgctatttggagctggtcatcctgttattaaggaacaaagagtagcaacaattcagggtctttcgggaacaggttcactgcgattagcagcggctcttatagagcgttatttccctggagcaaaagttgtgatctcatcaccaacctggggtaatcacaagaatatcttcaatgatgccaaagttccgtggtccgaataccgctactatgatccaaaaacaattggtttggattttgagggaatgatagcagatataaaggaagctccagaaggatccttcatcttgcttcacggatgtgctcacaacccaactggaattgacccaacaccagaacagtgggtaaaaattgctgatgtcattcaggaaaagaaccatatcccatttttcgatgttgcataccagggctttgctagtggaagccttgatgaagatgcagcatctgtgagattatttgctgagcggggaatggagttttttgttgctcagtcatatagtaaaaatttaggtttgtatgcagaaagaattggggcaatcaatgtcgtgtgctcctcagctgatgctgctacaagggtcaagagccagttgaaaaggattgctcggcctatgtactcgaatccaccagttcatggggcgagaatcgtggccaatgtagtgggtgatgtaactatgttcagtgaatggaaagcagagatggaaatgatggcaggaagaataaagacggttagacaagagctgtatgatagcctcgtttcaaaagacaagagcgggaaggactggtccttcattctgaagcaaattggcatgttctctttcaccggcctaaacaaagctcagagcgataacatgacggacaaatggcatgtgtatatgactaaagacgggaggatatcattggccggattatctctggccaaatgcgagtatcttgctgatgcgatcatcgactcataccataacgtaagctag

SEQ ID NO: 11 - DNA, 1754 bp, Oryza sativa
atggcgacgaccgcgaccctccccttctcctgctcctccaccctccaaaccctaacccgcaccatccccctccgtctccgcctccaccgccgccgcttcctccaccacctcccctccctcgctgccctcccgaggctcccgctcccgcgacctcccctcctcccccacgcgcgccgccacgtctcggcgtccgcggcgcccaacggcgcgtcctccgagggggagtacgactacgacctcttcaccatcggcgccgggagcggcggggtccgggcctcgcgcttcgcctccacgctctacggcgcccgcgccgccgtctgcgagatgcccttcgccaccgtcgcctcggacgacctcggtggagtcggcggcacatgtgtgcttcgtgggtgtgttccaaagaaattattagtgtatggatccaagtactctcatgagtttgaagagtctcatggctttgggtgggtgtatgaaactgatccaaagcatgactggaacactctgattgccaacaaaaatacagagctgcagcgccttgttggcatttacaagaatattttaaacaactcaggagttactctaattgaaggccgtggaaagatagttgatccacatactgtaagtgtagatggaaagctctacactgctaggaacatacttatagctgttggtgggcgaccatcgatgccaaatatcccaggaatagagcatgttatagattctgatgctgcactggatctaccttcaaaacctgagaaaattgcaatagtgggaggtggatatattgctctggagtttgctggaattttcaatggcttaaaaagtgaggtacatgtgtttattcggcagaagaaagttttaagagggtttgatgaagaggtcagagatttcatcgctgaacagatgtctctaaggggcatcacatttcatactgaacagagtcctcaagctataaccaaatcaaatgatggtttgctatctctgaagacaaacaaagaaactattggtgggttctcacatgttatgttcgcaacaggtcgtaaaccaaacacaaagaaccttggactagaggaggttggggtgaaattggacaagaacggagcaataatggttgatgagtattctcgaacctcagttgattcaatttgggcagtgggagatgttactgatagggtcaacctgacaccagttgcacttatggaaggtggtgcatttgcaaaaacagtgtttggtgatgaacctaccaaaccagattacagagctgtaccatctgccgttttctcccaaccacccatcggacaagttggtcttactgaagagcaggctattgaggagtatggagatgttgatatctatacagcaaacttcaggccacttagggcaactctctctggattacctgatcgcattttcatgaaactcattgtgtgtgctacaacaaacaaagttgtaggagtacacatgtgtggtgaagacgcacctgagataattcagggagttgcaattgctgttaaagctgggctgacaaagcaagattttgatgccactattggcattcacccaacatctgcagaggaatttgtcacaatgagaaatgcaactagaaaagttcggagaagcacaacagatgaggtagaatctaaagataaggttgttactcagaactagtacagataggaggcattctcagagggacttctctacttccgagcagctgatttacactgggcggacaatttttttttgtagatcgc

SEQ ID NO: 12 - DNA, 1491 bp, Oryza sativa
atggctaggaagatgctcaaggacgaggaggtggaggtggccgtcaccgacggcgggagctacgactacgacctgttcgtgatcggcgccgggagcggcggcgtccggggctctcgcacctccgcgtccttcggggctaaggttgcgatttgcgagctcccgttccatcccatcagctcggattggcaaggagggcatggtgggacgtgtgtgatacgtggttgtgtgcctaaaaagatactggtgtatggttcatctttccgcggagaatttgaggatgcaaagaattttgggtgggaaatcaatggggacattaacttcaactggaaaaggctgctggaaaataagactcaagaaattgttagactaaatggagtataccagaggattcttggcaattctggtgtgacaatgattgaaggggcaggcagtttggttgatgctcatacagttgaagtcacaaagccagatggttcaaagcaaagatatacagcaaagcacatattgatagcaactggtagccgagctcaacgtgtcaacattcctgggaaggagttagctattacttcagatgaggccttaagtttggaggagctaccaaaacgtgctgtaatccttggtggcggatatattgctgttgaatttgcttctatatggaaagggatgggtgcgcacgtagacttgttttatcgaaaagagcttcctctaagaggtttcgatgatgagatgaggacggttgttgcaagtaaccttgagggaaggggaatcagattacatccagggacaaatctatctgagttgagtaaaacagccgatggcataaaagttgtcactgacaaaggagaggagatcattgcagatgttgttctgtttgctacaggtcgcacaccaaactcccagaggttgaacttggaagctgctggtgttgaagttgataatattggagctataaaggttgatgattattctcgtacatcagtcccaaatatatgggctgtgggtgatgtaacgaaccggataaatttaacacctgttgcactgatggaggctacctgcttttctaaaactgtgtttggtggccagccaactaaacctgattacagagatgtaccttgtgctgttttctccatcccaccactatcagtagtgggcttgagtgaacagcaggctttggaggaagccaagagcgatgttcttgtttacacttccagcttcaacccaatgaagaacagcatatccaaacggcaggagaagaccgtcatgaaactggtggttgattcagagactgataaagtacttggtgcatcaatgtgtggaccagatgcaccagagattatccagggtatggctgtagcgctgaagtgtggagccaccaaggcgacctttgacagcactgttggtattcacccgtctgctgctgaagagtttgtgacaatgcggaccttgaccaggcgcgtgagcccatcatccaagccaaagacaaacttgtag

SEQ ID NO: 13 - DNA, 1428 bp, Agrobacterium tumefaciens
atggagaggctcgccaaattgccggtcttctgggggctcgagggtaagcgcgtcgttctgacaggcggatcggacggagcagcctggaaggcggaactgttgttggcctgcggggcgcagcttgatctttattgcgaggaaagcgggctttcggaaagcctcgcgacgctcgtcgcaaaaagcccgatgctgacatggcatgaccgttgctgggatgcagatattttcaaaggggcggaactggcgctggcagattgcgaagctgaagaggaagccggaagattttatcatgccgcgcgggcagcaggcgtgccggtcaatgtcatcgacaagccggaattctgccagttccagtttggttcgatcgtcaatcgctcaccggtggtggtgtcgatctccaccgatggtgccgcgcccattctggcgcaggccatccgccggcgcatcgagacattgctgccgctttcgctcaaggactggggcgctcttgcccagacaattcgagagcgcgttaatctgcggcttgcgcctggcgcggcacggcgctctttctgggaaaagtttgtcgaccgggcttttaccgaaagactggacgagggtagcgaagaacggctgctgaaagatgtagcgacgcggacggggctggcagaatcgggacgtggttttgtgacgctggtgggcgcagggccaggtgatgccgagcttttaaccctgaaagcggtgcgtgccctgcaggcggccgatgtcatcctgttcgacgatctcgtctcagcggaggtgctggaactggcgcggcgggaggccaagcgcatgctggtcggcaagcgcggcggccgcgaaagctgcaaacaggaagacatcaacgacatgatgatccgcttcgccaaggccggtagacgggtggtgcggctgaaatccggcgatccgatgattttcgggcgcgccggcgaggagatcgcggcgctggaagccgaaaatatcccggtcgaggttgtgcccggcatcaccgccgcaagcgccatggcctcacgtctcggcgtttccctgacccatcgcgaccatgcccaatccgttcggttcgtcaccggacattcgcggcagggaaagctgccggaaaatatcgactggcagtccctgtcgaacccttcggtgaccacggtgttttacatgggcgggcgaaccgccgcggacatccagtcttgcctgctcgcccacggcatgcccgcctcgacccccgtggttgtgatgatttccgtcagccgggtgaatgaacaacgctggtgcggttcgctcgcgcaactggttgctgcagtcgagaggctgggcgtgaacgaacccgtgctgatcggtgtcggggatgcgttccgttccgcttctgtcaacggcggagaacagaccgctgctgcgccttttcaaaaagccggctag

SEQ ID NO: 14 - DNA, 1071 bp, Zea mays
atggcctgcctcaccgacctcgtcaacctcaacctctcggacaacaccgagaagatcatcgcggaatacatatggatcggtggatctggcatggatctcaggagcaaagcaaggaccctctccggcccggtgaccgatcccagcaagctgcccaagtggaactacgacggctccagcacgggccaggcccccggcgaggacagcgaggtcatcctgtacccgcaggccatcttcaaggacccattcaggaggggcaacaacatccttgtgatgtgcgattgctacaccccagccggcgagccaatccccaccaacaagaggtacaacgccgccaagatcttcagcagccctgaggtcgccgccgaggagccgtggtatggtattgagcaggagtacaccctcctccagaaggacaccaactggccccttgggtggcccatcggtggcttccccggccctcagggtccttactactgtggaatcggcgccgaaaagtcgttcggccgcgacatcgtggacgcccactacaaggcctgcttgtatgcgggcatcaacatcagtggcatcaacggggaggtgatgccagggcagtgggagttccaagtcgggccttccgtgggtatttcttcaggcgaccaggtctgggtcgctcgctacattcttgagaggatcacggagatcgccggtgtggtggtgacgttcgacccgaagccgatcccgggcgactggaacggcgccggcgcgcacaccaactacagcacggagtcgatgaggaaggagggcgggtacgaggtgatcaaggcggccatcgagaagctgaagctgcggcacagggagcacatcgcggcctacggcgagggcaacgagcgccggctcaccggcaggcacgagaccgccgacatcaacacgttcagctggggcgtggccaaccgcggcgcgtcggtgcgcgtgggccgggagacggagcagaacggcaagggctacttcgaggaccgccgcccggcgtccaacatggacccctacgtggtcacctccatgatcgccgagaccaccatcatctggaagccctag

SEQ ID NO: 15 - DNA, 1032 bp, Arabidopsis thaliana
atgttcggtagaggaccctcgaagaagagcgacaacactaagttctacgagatcttaggtgttcctaagagcgcttcaccagaagatctcaagaaagcttacaaaaaagccgctatcaagaatcatcctgataagggtggagatcccgagaagtttaaggagttagcacaagcttatgaagtgcttagtgacccggagaagcgtgagatttatgaccagtatggagaggatgcactcaaggaaggaatgggtggtggaggaggtggacatgatccatttgatattttctcatccttctttggtggaggcccctttggaggtaataccagccggcaaaggaggcagaggcgtggtgaggatgttgttcatcccttgaaggtatctcttgaggatgtgtaccttggtacaatgaagaagctttcactttctaggaatgctctctgctctaagtgtaacggaaagggatcaaaatctggagcctccttgaaatgtggagggtgtcagggatctggtatgaaggtgtctattaggcagcttggacctggaatgatccagcagatgcagcatgcatgtaatgaatgcaaagggacaggtgagaccatcaatgatcgggacaggtgtccacaatgcaaaggagacaaggtcattcctgagaagaaggtgcttgaagtgaatgtggagaagggaatgcaacacagtcagaagatcacatttgaaggacaagcagatgaagcgcctgacactgtcactggagatatagtgtttgtccttcagcagaaagagcacccaaagttcaagagaaagggagaagacctctttgtggagcacacactttctctaaccgaagctttgtgtggcttccaatttgttctgactcacttggatggcagaagtcttctcattaaatctaatcctggggaggtcgtgaaacctgattcatacaaggcaataagcgatgaaggaatgccgatataccagaggccattcatgaaggatgaggaggaaggcacaagctcaaagagaggcttatag

SEQ ID NO: 16 - DNA, 1725 bp, Zea mays
atggcagccgatccttcgtcctcttccacggggcaacaaacggcggacatccgcgcggccccgcccgaggactcgaggcagatggcaatgagcgggccgctcaatgtccggggcgaccggaggccgccgccgatgcagagggccttcagccggcaggtctcgctcggcagcggcgtgacggtgctgggcatggacagagcggggaggagcggtggcgcaaggggccaacggaccctcccccgcagtggtaggagcctcggggtgctcaaccacagcggcggcttggtccaggccgccggcgacggcgccgcgcgcagggttggcgacttcagcatgttccggaccaagtcgacgctgagcaagcagaactcgatgctgccgacgaggatcagggagtccgacctcgaactgcccacgcacgtcgaagacccgcagtccgccagcagcaggccggcggaggacccgctcaacaagagcgtccccgccggccgctacttcgcggcgctccgtggccctgagctcgacgaagtccgcgatactgaggacatcctgttgcccaaggacgaggtgtggccgttcctgctgcggttcccgatcggctgcttcggtgtgtgcctgggcctcggcagccaggccatcctgtggggcgcgctggcggcgagcccggcgatgcgcttcctccacgtcacgcccatgatcaacgtcgcgctgtggctgctggcggtcgccgtgctcgtcgcgacgtccgtcacctacgcgctcaagtgcatcttctacttcgaggccatccgacgcgagtacttccacccggtccgcgtcaatttcttcttcgcgccgtggatcgcggccatgttcgtgaccatcggcctaccccgctcctacgcgcccgagcggccgcacccggccgtgtggtgcgccttcgtcctgccgctcttcgcgctcgagctcaagatatacgggcagtggctgtccggcggcaagcggcggctgtgcaaggtggccaacccgtcgtcccacctctcggtggtgggcaacttcgtcggggccatactggcggcgagggtcgggtggacggaggccggcaagctcctgtgggccatcggggtcgcgcactacatcgtcgtgttcgtcacgctgtaccagcggctgcccaccaacgaggcgctgcccaaggagctgcacccggtgtactccatgttcatcgccacgccgtcggccgccagcctcgcctgggccgcgatctacggcagcttcgacgccgtggcgcgcaccttcttcttcatggccatcttcttgtacctgtccctcgtcgtgcgcatcaacttcttccgggggttccggttctccctcgcgtggtggtcgtacacgttccccatgaccacggcgtcgctggccaccgtcaagtatgccgaggctgtgccgtgcttcgcgagcagggccctcgcgctgagcctctccctcatgtcgtcgaccatggtgtcgatgctgctcgtgtcgacgctcctgcacgcgctcgtctggcgatcgctctttcccaacgacctggccatcgccatcaccaaggaccggcaaaacggcgcggtgaagccgaatgacagggggaagagggccagcaagagagtgcacgacatcaagcgatgggccaagcaggcgcccctctccctcgtgtcctcaatcaccaagagccactcggcggacaaggaggaagaagagagaactgaatag

SEQ ID NO: 17 - DNA, 738 bp, Zea mays
atggctcaggaggacgtgcacctggacgatgccggcctggcgctgtgcctgtccctccacggtaccagcagcagccggctgagcacggaggcgccgcgcacgctggagccgccgtcgctgacgctgagcatgccggacgaagcgaccgcgaccgcgaccggcgggtccggcggcagcggcggggccgcgcgcagcgtgtcgtcgcggtcagtggagggcgtgaagcgggagcgcgtggacgacgccgagggcgagcgggcgtcgtcgacggccgccgcggcgcgggtctgcgccggcgccgaggacgacgacgacgggagcacgcggaagaagctgaggctgaccaaggagcagtccaagctcctggaggaccgcttcaaggaccacagcaccctcaacccgaagcagaaaatcgcgttggcgaagcaactgaagctgaggccacggcaggtggaggtgtggttccaaaacaggcgagcaaggacgaagctgaagcagacggaggtggactgcgagctgctgaagcgctgctgcgagtcgctgagcgaggagaaccggcggctgcagcgggagctacaggagctccgcgcgctcaagctcgccggcccgcacccacaggcgccgtcgtcgtcgcccgccgccgcgacgcagggcgtgccggtgccggtgccgccgccgttgtacgtgcagatgcagatgcagctcagcagctgccgatgctgccggccgccacgctag

SEQ ID NO: 18 - DNA, 744 bp, Zea mays
atggagaaagaagaggggttcgggaagtcatggcttggcctggggatcggcggcggtggccgcgatctgaatctgatgaagcggagccgaccactacgaccggtgcggctggacctgctgttcccgccgagtgtggagggaggagaagctgccgcgaggagcaggaaggctggtgcaggtgcactgcggaatatgtcgttgaagcaggtcgcaggcgacgacgatggtgggcagtcgtcgcacggtggtccgagccccagcgacgacgacgacggcgcaggcgcgcggaagaagctccggctcaccacggagcagtccaagctgctcgaggacaccttccgcgcccacaacatactctcccacgctcagaagcatgaggtggcgcggcaggtggatctaagcgccaggcaggtggaagtgtggttccagaacaggagggcaagaacaaagctgaagcaaacggaggtggactgcgagaccctgaggcgctggcgcgagagcctggcagacgagaacctgcggctgaggctggagctggagcagctgcagcggtgggcgaccgccgccgctggtcagtcctccgcgtccccgtcgccggccacggccacggcgagcgtctgtccgtcgtgcgacaaggtcgtcgtcgtcaccgtgacgagctgtggggagacaagcggcaagagctccaccagcagctactcctccagtcctcctcttgacatgctcgatcgatcggttcaatag

SEQ ID NO: 19 - DNA, 888 bp, Zea mays
atgatgccccaggccagcgctagcctcgacctcggcctcagcctgggcctcaccctcacctcccagggcagcctctcctcctccaccaccaccgccggctcctcctccccctgggcagccgcgctcagctccgtcgtggccgacgtcgccagggcgcggggtgacgcgtacgcgcagcaccacgccggcgccgcgatgacgatgcgcgcgtccacgtcgcccgacagcggcgacaccaccaccgccaagagggagagggagggggagctcgagcgcaccggctccgccggaggcgtccgcagcgacgaggaggacggcgcggacggcggcgccggcgggcgcaagaagctcaggctctccaaggaccaggccgccgtcctcgaggagtgcttcaagacgcacagcacgctcaaccccaagcagaaggtgcagctggccaaccgcctgggcctccggccgcggcaggtggaggtgtggttccagaaccgccgcgcgcggaccaagctgaagcagacggaggtggactgcgagtacctcaagcgctggtgcgaccgcctcgccgacgagaacaagcgcctcgagaaggagctggccgacctcagggcgctcaaggccgcgccgccgtcgtcggccgccgcgcagcccgcctcggccgccgccaccctcacaatgtgcccgtcctgccgccgcgtcgcggccgccgctagccaccaccaccagccgcccccgccgcaatgccaccccaagcctaccgtcgccgccggtggcggcagcgtcgtgcccaggcccagccactgccagttcttcccggccgccgccgttgaccggacgagccagggcacgtggaacaccgccgcgccgccgctcgtcaccagagaactcttctag

SEQ ID NO: 20 - DNA, 588 bp, Oryza sativa
atgggggaggaggcggtggtgatggaggcgccgaggcccaagtcgccgccgaggtacccggacctgtgcggccggcggcggatgcagctggaggtgcagatcctgagccgcgagatcacgttcctcaaggatgagcttcacttccttgaaggagctcagcccgtttctcgttctggatgcattaaagagataaatgagtttgttggtacaaaacatgacccactaataccaacaaagagaaggaggcacagatcttgccgtctttttcggtggatcggatcaaaattgtgtatctgcatttcatgtctttgctactgttgcaagtgctcacccaagtgcaaaagaccaaggtgcctcaattgttcttgcagctcatgctgcgacgagccatgctgtaagccaaactgcagtgcgtgctgcgctgggtcatgctgtagtccagactgctgctcatgctgtaaacctaactgcagttgctgcaagaccccttcttgctgcaaaccgaactgctcgtgctcctgtccaagctgcagctcatgctgcgatacatcgtgctgcaaaccgagctgcacctgcttcaacatctga

SEQ ID NO: 21 - DNA, 1074 bp, Oryza sativa
atgcagaagcagcacgccgccgactcggccgcgctcgtcgcggccatgggcgaggtgcaccgcctccgcgtgcagctggccgcggcggcgcgcgccgaccgcaagcaggacgtggtggaggcgatggccaccatcgacgagctcagggtgaagctcaaggcgagcgaggaggccgaggcgcaggcgcgcgccttgcacgaggagtgcaagcagcagctggagacgagccgtgccaccatcgactcgctgctcacggacggctccaagctcatggactccttcagcctcgtggtcaaggagctcgaggagtcacgagccaaggtgaaggcactcgaggaggagatcgcggagacgtcggcggcaaaggccggcgagcgttgcaactgctcggcgtcggcgtcggcatcggaggtcgctgagctgaggtcggaattggagtccacggaggccaggttccaagaagagcgcatcctgagcacagtggagacgcagtgcgcctacgagctcatggaccagataaaaatggagtccgactcgcggcacggcaagctcgccgcggcgctcgagagcaccaagtccgaggtcatcttcctcaaggcgagcctcttcgacaaggactccgagctgcggcgcgccctggacgcgaacgagaagctccaatccgagacgagaacggacaacgagctgaaggagcagctgcagggcgcgctcctggagaacgggcagctgaagcgcgagctgcagcagcacacctccgagaagaaggcctcggcgaaggcgacggacgccgccgacgcggcggcggaggcggcgaagaagggggagatggaggccgagctgaggcgtctgcgggtgcaggccgagcagtggaggaaggccgccgagaccgccatggcgctgctcacggtgggcaagggcggcaacgggaaggtggtggaccggagcgagtcgcttgaaggaggcggcggcggcggcggcaagtacgccggcctgtgggacgagctcgacgacgacgcggcggccaggaagaacggcaacgtgctcaggcggatcagcggcatgtggaagaaatag

SEQ ID NO: 22 - DNA, 477 bp, Arabidopsis thaliana
atgggagagattgggtttacagagaagcaagaagctttggtgaaggaatcgtgggagatactgaaacaagacatccccaaatacagccttcacttcttctcacagatactggagatagcaccagcagcaaaaggcttgttctctttcctaagagactcagatgaagtccctcacaacaatcctaaactcaaagctcatgctgttaaagtcttcaagatgacatgtgaaacagctatacagctgagggaggaaggaaaggtggtagtggctgacacaaccctccaatatttaggctcaattcatctcaaaagcggcgttattgaccctcacttcgaggtggtgaaagaagctttgctaaggacattgaaagaggggttgggggagaaatacaatgaagaagtggaaggtgcttggtctcaagcttatgatcacttggctttagccatcaagaccgagatgaaacaagaagagtcatag

SEQ ID NO: 23 - DNA, 750 bp, Solanum lycopersicum
atggctggcggcgtagctattggaagttttagtgattcattcagcgttgtctctcttaagtcctatcttgccgaattcatctccacactcatctttgtcttcgccggagttggttccgccattgcttacggcaagttgacaacaaatgctgcacttgatccggctgggcttgtagctattgcagtttgccatggatttgctctattcgtagccgtttcgatttccgctaacatctccggtggtcatgttaaccctgcggtcacctgtggattaaccttcggcggacatattacctttatcactggctccttctacatgcttgctcaacttaccggtgcggctgtagcttgcttcctcctcaaattcgtcaccggaggatgtgctattccaacccatggagtgggagctggtgtgagcatactagaaggactcgtgatggaaataataatcacatttggtttagtttatactgtgttcgcaaccgccgctgacccgaagaagggttcattgggcacaattgcaccgattgcaattggtctcattgttggagctaatattttggctgccggaccattctccggtggatcaatgaacccagctcgttcatttggacctgcaatggttagtggtaactttgagggtttctggatctactggattggtccattagttggtggtagtttggctggtcttatttacacaaatgtgttcatgacacaagaacatgctcctttatccaatgagttctag

SEQ ID NO: 24 - DNA, 1221 bp, Arabidopsis thaliana
atgtcacttagcccgcgagttcagtccttaaaaccttccaagactatggttataaccgatcttgcagccactcttgttcaatccggtgttccggttattagactagctgcgggagaacccgatttcgacactcccaaagtcgtagctgaggctgggatcaacgcgattcgagaaggttttactaggtatacgttaaatgcaggtattacagaactcagagaagcaatatgtcgaaagctaaaagaggagaatggattgtcttatgcgcctgatcagatcttggttagtaatggagctaaacaaagtctcttacaagcagtgcttgcagtttgttctcctggagatgaagttataattcctgcaccgtattgggtgagttacacagaacaggcgagattggctgatgcaacgcccgtggttattcctaccaagatttctaacaattttttgttggatccaaaggatcttgagtctaaattgactgaaaaatctagacttcttattctctgctctccttccaaccctactggatctgtttaccccaagagtttgctcgaagagattgcacggatcattgctaagcatccaagacttctggtgctttcggatgaaatatatgaacacattatttatgcacctgcaacacacacaagctttgcttctttgcctgacatgtatgaaagaactttgacagtaaacggtttctcaaaggctttcgcaatgacgggttggaggcttggatatcttgctggtcctaaacatattgtggcagcttgcagtaaattacaaggccaggttagttcaggagctagtagcattgctcagaaagcaggtgttgctgcgcttgggttaggcaaagctggaggagaaacggttgcagagatggttaaagcttatagagaaagacgagatttcttggttaaaagcttaggtgatatcaaaggtgttaagatctctgaacctcagggagctttttatctctttattgacttcagtgcttactatggatcagaagctgaaggttttggtttgatcaatgattcgtcgtctcttgcactatactttcttgacaagtttcaggttgcaatggttcctggtgatgcttttggagatgatagttgtatccgaatatcttatgccacatctctcgacgttcttcaagcagctgttgagaagatcaggaaagcccttgagccactccgtgccactgtctccgtttag

SEQ ID NO: 25 - DNA, 849 bp, Gossypium raimondii
ggggagaaaaagttggctacaatttataatgtagtcgctgtcataagaggattggaggagccagatcgttatgttttgatggggaatcatagagatgcttggacatatggtgctgttgaccccaatagtgggactgcaacactccttgatattgctcgaagatatgcccttttgatgcgaaagggttggaatcctcggaggacaatcattttttgcagttgggatgctgaagaatttggaatgatcggttccacggagtgggttgagcagaaccttgtaaatcttggtgctaaagctgtggcatatcttaacgtagattgtgcggtgcaagggcctgggttttttgctggcgcaactcctcagctagataatcttatttttgaggtcacaaagaaggtccaggatcaggattcagaggttgtagctacaatatatgaaaaatggaaaaccatgaacggaaacaatattcaaagactcagtggcgtagattctgattttgcaccattcttgcaacatgccggggttccttctgtcgacatatattatggaagagatttccctgtatatcatactgcattcgattctttcaactggatgataaacaacgcagatccattcttttggcgtcatgtggctgtggctggagtttggggtcttctaggccttcaccttgctgatgatccagttctacctcttgattacctctcctatgctaaacagttgcaggtatggggttattctctccttgtgtttgtggatattgtcaagtgttcccaaccatttccactattactgttcttctacaaggttttggtggggctgttgattgttaacccttggttacaataa

SEQ ID NO: 26 - DNA, 1065 bp, Zea mays
atggagctggggctgagcctgggcgacgcggcagtgccggacgccggcagggcggctccggagctgggcctggggcttggggtcgggattggatccaacgccgccggaaccggcaggggaagcaaggcggcggggacgacgggaactactgggtggtgggcggcgccggccacaccggagtcggcagtgcggctcagcctcgtgtccagcctcggccttcagtggccacctccggacggcggcatctgtcatgtagggcgcgacgaggcgccggcgcgcggcttcgacgtgaaccgggcgccgtcggtggcggggagcgccctggcgctggaggatgacgaggaggagccgggcgccgcggcactgtcgtcgtcgcccaacgacagcgcgggctccttcccgctggacctgggaggcccacgcgcccacgccgagggcgccgcggcgcgggccggcggcgagcggtcctcgtctcgcgccagcgatgaggacgagggcgcgtccgcgcgcaagaagctgcgcctctccaaggagcagtctgcgttcctggaggagagcttcaaggagcacagcaccctcaaccctaagcagaaggcggcgctggcgaagcagctcaacctccggccgcgacaggtagaagtctggttccagaaccgccgagccaggacgaagctgaagcagacggaggtggactgcgagtacctgaagcgctgctgcgagacgctgacggaggagaaccggcggctgcacaaggagctcgcggagctgcgcgcgctcaagacggcgccgcccttcttcatgcgcctcccggccaccaccctctccatgtgcccctcctgcgagcgcgtcgcctccggccccagccctgcctccacctcggcacctgcgtcgtccacgccgcctgccacagccgccaccaccgccatctcgtacgctgcagcagccgccgcacccgtgcgagccgaccaccggccctcgtcgttcgccgcgctgttcgcggcgacccgcagcttcccgctggcgtcccagccgcggccgcccgcgccggcgagcaactgcctgtag

SEQ ID NO: 27 - DNA, 882 bp, Thellungiella halophila
atgctcaaggtccctgaacaccaagttgctggtcacattgccatagatgggaagcttggtccgctcgtagacgaccaaggccgattcttcaagccacttcaggatgatgctcgtggtgaaaacgaggctaagttctatgagtctttctcggcgaacaagaatgttccagatcacatccatagatacttcccggtgtatcacggtactcagttagtcgaagcatctgatggatctggcaagcttccacacatggttcttgaggatgttgtttccgagtactcaaatccgtcgataatggatgttaagattggatctagaacatggtatccggatgtgtcggaagaatacttcaagaaatgcataaagaaagatagagagaccactacggtttcgttggggttcagggtttcaggttttaagatttttgaccaccaagaatcgagtttttggagacccgagaagaaggttgttcttgggtacaaagtggatggtgctagattggctctgaagaagtttgtgtcatcgaactctcctgttgagtctaagtcaatgccaaactgtgcttttgcgtcagaggtttatggcggtcctaatgggatcttagcgcaattgttggagcttaaggcttggtttgaaacccaaacgatctaccatttcaattcttgctcgattctgatggtgtatgagaatgattcgatgttgatgaaaggaggggatgatgcgcagatgcctcgggcacaagtaaagctggtggatttcgctcatgttcttgatggaaatggtgtcatcgaccacaatttcttgggtggagtctgctctttcataaaattcatccaagatattcttgaaaccgacacttcccagcttgaaaacgggcactag

SEQ ID NO: 28 - DNA, 1569 bp, Sesbania rostrata
atgggatatgaaaccagaaggctctcagatgagtatgaggtttcagatgttctaggaagaggtggattttctgttgtcagaaaaggtaccaaaaaatcaagcagtgagaaaaccttagtagccatcaaaacactgagaaggttaggtgcctctaataacaacccttctggtttaccaaaaacaaaaggtggagagaaaagcatagcaactatgatggggttccccacatggagacaagtttcagtctcagatgccttgttgaccaatgagattcttgtcatgaggaggatagtggaaaatgtttcacctcaccccaatgtgattgacctctatgatgtgtatgaggactcaaatggggttcatcttgtgcttgagctttgttctggtggggaattgtttgataggattgtggcacaagataggtactcagagactgaagctgcagctgtggttcgccagatagcagcaggattagaggctattcataaagctaacattgttcatagggacttgaagcctgagaattgcctttttttggataccaggaaggactctcctctcaagatcatggactttgggttgagttctgttgaagaatttactgaccctgttgttggtttgtttggatccattgattatgtttcaccagaggctctttctcaaggaaagataactactaagagtgacatgtggtctctaggagtaattctatatatcttactctctgggtatccacctttcattgctccgtctaatcgccaaaaacaacaaatgatagtgaacgggaatttcagtttctatgagaagacttggaagggcatttcccaatcagcaaagcaattgatttcaagtcttctgactgttgatcctagcaagagacccagtgctcaacagcttctgagtcatccatgggttataggtgagaaagccaaagatgatcaaatggaccctgaaattgtctcaaggctgcagagctttaatgcaagacgcaaactgcgtgcagctgcaattgctagtgtttggagctccacagtcttcctcagaaccaaaaaactgagatccttggtaggaacccatgatctcaaagaagaggaaattgaaaacctcaggatacatttcaagaagatatgtgcaaatggagacaatgccactctctctgagtttgaggaggtgctgaaagcaatgaatatgccatcattgatccctctagcacctcgtatatttgacttgtttgacaacaaccgtgatggaacagttgacatgcgagagatactatgtgggttttctagtctcaagaactccaaaggagatgatgctctccgtttgtgcttccagatgtatgacacagatcgatccgggtgcatcacaaaggaagaagtagcatctatgctgagagctttgccagatgattgtcttccagctgatatcactgaacctggcaaattggatgagatatttgatttaatggatgcaaatagtgatggaaaagttacctttgatgaattcaaagctgctatgcagagagatagctctcttcaagatgtagtcctctcttctcttcgcccatag

SEQ ID NO: 29 - DNA, 276 bp, Arabidopsis thaliana
atgataaaactactatttacgtacatatgcacatacacatataaactatatgctctatatcatatggattacgcatgcgtgtgtatgtataaatataaaggcatcgtcacgcttcaagtttgtctcttttatattaaactgagagttttcctctcaaactttaccttttcttcttcgatcctagctcttaagaaccctaataattcattgatcaaaataatggcgattttgccggaaaactcttcaaacttggatcttactatctccgttccatag

SEQ ID NO: 30 - DNA, 1686 bp, Zea mays
atgctgtctgaagatttcatagtcgcagatattgctattcatcctagacatgctcggataatgaaaccacatcagttggaaggtttcaactttttggttaagaatttgattggagacaagcctggaggttgcattctagctcatgccccaggtacagggaaaacatttatgcttataagcttcattcagagcttcatggcaaggtatccatctgcaaggcctcttgttgtgctgcccaaagggattctaggtatatggaagacagaagttaaacgatggcaagtgcaggatataccagtgtacgatttttactctgtcaaggctgaaaaaagagtagaacagttgcaaatcctcaaatcttgggaagacaagatgagtatactatttcttggatacaagcagttctccacaatcgtcactgatgatgggggcagcaatgtcacagctgcatgtcgagacaggctgcttaaggttcccaaccttctgatacttgacgaaggacatacacctagaaatcgggagactaatgtactcgaatcactaaatagagtggaaacaccacgcaaagtggttctttcgggtacacttttccagaatcatgttgaagaagtgtttaatatcttgaatctcgttcgcccaaagtttctcaggatggaatcatcccgtcctactgccagacgtataatgagtcaagttgaaatagttggtagaagttcgaaagggcttgctgatggcgcattcactaaggcagttgaagaaaccttattgaatgatgagaacttcaagagaaaagctcatgttattagaggtcttagagaactaacaaaggatgttcttcactattataagggtgatatcttagatgaactacctggcttagtagacttcagtgtgtttttgaagctcacacccaaacagaaagacattatttataagttggaagcacatgatcggttcaaaagaaatgcagtagggagtgcactgtacattcatccatgtctttcagaactttcagaggttaatgctgagcatagggctaacacctttagagatgatttagttgatagtctggtagattctatcactgtgagagatggcgtgaaggccaattttttcatgaatatcctgtcacttgctaattctgcaggagagaaagtgctagctttcagtcaatatatatctcccatgattttctttgaaaggctgctggtgaagaagaaaggctggcatgtggggaaagagatctttatgatctctggtgatactagccaagaagacagagaattggcaacggaccattttaacaactctgctgacgcaaaaattatgtttggttctatcaaggcatgtggggagggtatctccctcgttggtgcgtcgagagttgtcattctggacgtacacctgaacccatctgtcacccgtcaagcgattgggcgtgcattcaggcccggacagcagaagaaggtgtttgtgtacaggcttgtagctgccgattctgacgaggtaaaggtccatgagacagcattcaagaaagaagtcataccgaagctgtggttcgaatggagcgagcactgtactacggaagacttcaaacttggtcaaattgatattgatgactctggtgacgaactgttggatactaaagcaatccgcaaggatatcaaagcgctgtatagaaggtag

SEQ ID NO: 31 - DNA, 873 bp, Arabidopsis thaliana
atggtcgtaacagctttgtggtgtgggattctcataagttctcaacaactctccttccacgttacttcaagcatagcaatttctcaagttttattcgtcagctcaatacttatatgggtttcctatgagtcttcagctataaagggattcagaaagattgatccagatagatgggaatttgcaaatgaagggtttttagcaggacaaaagcatctcttgaagaacatcaaaagaaggaggaacatgggtttgcagaatgtgaatcagcaaggatctgggatgtcatgtgttgaggttgggcaatacggtttcgacggggaggttgagaggttgaagagggatcatggtgtgcttgtagctgaggtagttaggttgaggcaacagcaacacagctccaagagtcaagttgcagctatggagcaacggttgcttgttactgagaagagacagcagcagatgatgacgttccttgccaaggcgttgaacaatccgaactttgttcagcagtttgcggttatgagtaaagagaagaagagtttgtttggtttggatgtggggaggaaacggaggcttacttctactccaagcttggggactatggaggagaatttgttacatgatcaagagtttgatagaatgaaggatgatatggaaatgttgttcgctgcagcaatcgatgatgaggcgaataattcgatgcctactaaggaggaacaatgtttggaggctatgaatgtgatgatgagagatggtaatttggaagcagcgttggatgtgaaagtggaagatttggttggttcgcctttggattgggacagccaagatctacatgacatggttgatcaaatgggttttcttggttcggaaccttaa

SEQ ID NO: 32 - DNA, 636 bp, Physcomitrella patens
atggctttgagtcagagttctacgtgtagtcaagtgagcggtcttgtagtgcacgccgatctggcccggccgcaatcgcctaagacacaggctccgatgagtgctgttcctgtcaaggcggacacggcgtttcaaggaactgcgctgcgatccgttggtcgtcagacgcgatccatggcggctcctaatgttgccttgaaggacctcgtggcatcgagagatgcggaggtaggctcctcagtgtcgaagttggttagtgaagggagcgaagatttggatagcattgctactacttccagcgacttgagtgaggttgtggatgtcgttgaggaagacgcgggtggggctaacattcgtgtgaggaaagcctctggaaaggcaggcactaggacctccaggaggcgggcgttggtgatgtgcttggcgttgggcatggtcaggccaatctctggcaatgccactggtgggttgcaggcaggaaatctgcgcaggacgacttccaccaatctccggcggtcggcttcctccagcttcactgtaagcggcaaccttcaaagccaagtgtctattgcatcttctctcaaggctgcgaatctgctggacgataagctcaagaacaacgttcctacgctttga

SEQ ID NO: 33 - DNA, 753 bp, Physcomitrella patens
atggcggacgaatacgggcgggaaaggattagagacgctgttgaagggcttggtgaggacggacctgtagtaggcggtgaggttacggaccgtggtttgtttggaagacacggaagacatcacggatacaacagcggatacagtgaagaagatgcgtttgcctctgagctcggaggtccatatggacgtcgacctccacctggagctgtggtttatgaaggagaaggtggctttggcgatggatatggacgtcgtcccccagtaatgccatatgagggagtaggaggaggctatggcggaggttatggaaatgaatatccaccagatgttgctggaggtggctatggccggcatggttatgcaggtgaggactatggtcgtcgtcctggtcctcccatgtacgtcgaagcgccggttgagaattccgatctcggcactggtttggtagactccaatatccgaactgagccagattacggcgctggctacggtcgtccggatggcactagcgcatacgaagttcagggacgtcacggtgggaagcacggccacttgagtaaagaggaacgagaggagcttgaggatgagcgcaagcacaagcattatgctgaagcggcggctgctgcggccctcggctacggactctacgagcgtcatgagaaaagagacgcggaggataggctggaagaactcggctacgattctgacggcaagaagaaacaaggccaccacttcttccgctccgattcctaa

SEQ ID NO: 34 - DNA, 633 bp, Physcomitrella patens
atggcgttaaattctctggcaagtacttctgtgatcagaggaattgctctgcctgctccgttttgtgattctacacagctgcggcgacaagctgcaagcccctttgtttcccgcccaaggtcgtatagaacggtcgtgcggagctcgaggttaccgctgaatccaaaggaggctcgagaaatggctgaaggtcgagaacctgagaggcagaatgaacgtggtggtaatggcggacccaaccccttcagatttttccagaatttcaaggacggcctatttcaggaccacaagagactacagaaggagaagagcctgcctaaaggcgacctcttgtacacggttgagaaaggcgatacattgtacgctatctctgaaagacacgaatgttctcttgagcttcttatggaggccaatggcattgaagatcctcacaacttaagcgttggacaggagatctggattccacggacttatcagattaagaagggtgacactttgtactcaatctcgaaacattatggcgtgagtattgaggctattcaggccgccaatggaatcgacgaccccaattttattcatgaaggagaccatatatgtcttccagaaaagactgctcacgaggactcagactga

SEQ ID NO: 35 - DNA, 1089 bp, Arabidopsis thaliana
atggataacttcttaccctttccctcttctaacgcaaactctgtccaagaactctctatggatcctaacaacaatcgctcgcacttcacaacagtccctacttatgatcatcatcaggctcagcctcatcacttcttgcctccgttttcatacccggtggagcagatggcggcggtgatgaatcctcagccggtttacttatcggagtgttatcctcagatcccggttacgcaaaccggaagtgaattcggttctctggttggtaatccttgtttgtggcaagagagaggtggttttcttgatccgcgtatgacgaagatggcaaggatcaacaggaaaaacgccatgatgagatcaagaaacaactctagccctaattctagtccaagtgagttggttgattcaaagagacagctgatgatgcttaacttgaaaaataacgtgcagatctccgacaagaaagatagctaccaacagtccacatttgataacaagaagcttagggttttgtgtgagaaggaattgaagaacagcgatgttgggtcactcgggaggatagttctaccaaagagagatgcagaagcaaatcttccgaagctatctgataaagaaggaatcgttgtacagatgagagatgttttctctatgcagtcttggtctttcaaatacaagttttggtccaataacaagagcagaatgtatgtcctcgagaacacaggagaatttgtgaagcaaaatggagctgagataggagactttttaacaatatacgaggacgaaagcaagaatctctacttcgccatgaatggaaattcgggaaaacaaaatgaaggaagagaaaatgagtcgagggaaaggaaccactacgaagaggcaatgcttgattacataccaagagacgaagaggaagcttccattgcaatgctcatcggaaatctaaacgatcactatcccatccctaacgatctcatggacctcaccactgaccttcagcaccatcaagccacgtcctcaatgacacctgaggatcacgcgtacgtgggttcatccgatgatcaggtgagctttaacgactttgagtggtggtga

SEQ ID NO: 36 - DNA, 1149 bp, Corynebacterium glutamicum
atgaccgcaacctacaccactgaaaccgccatcaatttcttgttcttgagcgaaccggacatgatcgcggccggagtcaaagacgtcgcgcaatgcgtcgatgtcatggaggaaacgctcgtgctcttggcgcagggcgactacaaaatggccggtttgaactccaactcgcatggcgcgatgatcaccttcccggaaaacccagaatttgaaggcatgcccaaggacggccccgaccgccgattcatggcgatgcccgcatacctcggcgggcgattcaaaaacaccggcgtgaagtggtacggatccaacgcggaaaacaaggcctcaggcttgcctcgctcgatccacaccttcgtcctcaacgacacggtcaccggtgcaccgaaggccatcatgtccgcgaacctgctgtccgcctaccgcaccggcgcggttcccggcgtgggcgtgaagcacttagcggtcgccgacgcgacaaccttggctgtcgtcggacctggtgtcatggcgaaaaccatcaccgaagcgtgcatcgcagagcgcccaggaatcaccaccatcaagatcaagggacgcagcgaacgcggcatcaacgcctttgcaacatgggcgttggaaaaattccccgagatcgaagtggtcgccgtcggatctgaagaagacgtggtcaaagacgccgacatcgtcatcgccgccaccaccacggacgccgccggctcctccgccttcccatacttcaaaaaagaatggctcaagccgggcgcattgctgctgcttccagccgccggtcgcttcgacgacgcttatttgcttgacgacgcccgcctcgttgttgactacatggggctctacgaagcctgggcagaagaatacggcccacaggcctaccaactactcggcattccaggaacccactggtacgacctggcgctgcaaggaaaactcgaccttgcaaagatttcccagattggcgatatctgctccggcaagctacccggacgcaccaacgatgaggaaatcatcctctattccgtcggcggcatgccagtagaagacgtcgcctgggcaacccaagtgtatgaaaacgccctggaaaaaggcgtcggcaccacattgaacctgtgggaatcacccgcactggcttga

SEQ ID NO: 37 - DNA, 2133 bp, Arabidopsis thaliana
atggcagccacgcttccactctgtgctgccctccgatctcccgtctcttcccggagattcgctccaattcacaaaaccgacgttccttttcagttcaatgtcgtcctttcaccgtttttcggttccgtcgctattggcggtagaattttcccgcgtttaccggcggcgaagcaggagactgatcaggatgaggttggatttgatcagcagccgtctcaggagcttgcgatagcgtcggcttgtttggttggtgttctcactggagttagtgtggttctattcaacaactgtgttcacttgcttcgagacttttcctgggatgggattcctgatcgtggagcttcgtggcttagagaggcaccgatcggttccaattggttgcgtgttatccttgttccgactatcggcggtttggtggtgagcatcctcaatcagcttcgagaatctgctggaaaatctactggagattctcattctagtctcgatcgcgtaaaggcagtgttgcgtcctttccttaagactgttgccgcatgtgtgacgcttgggactggaaattcgctggggccggaaggtccaagtgttgaaattggagcgtcaatcgctaaaggtgtgaattctctgttcaataaaagtcctcagactggcttctcacttcttgccgctggctcagctgctggcatttcctctgggttcaatgcagctgtggctggatgcttctttgcagttgaatccgttttgtggccttcttcatcaactgattcatcaacttcacttccaaacacaacttctatggttattcttagtgctgttactgcttctgtggtttccgaaatcggtctcggctctgaacctgcgtttaaggttcctgactatgacttccgctctcctggagaacttccactctatcttttattgggcgctctgtgtggcttggtctcgttggcattatctcgatgtacatcatccatgacatctgctgttgacagtcttaacaaggatgctgggataccaaaggctgtatttcctgtaatgggtggattaagtgttggtatcatagctttggtataccctgaagtattatactggggttttcagaatgtggatattttgttggagaaacgtccatttgtgaagggtctttcagctgatcttttgcttcagctggtagcggtcaagatagctgcaaccgcatggtgtcgggcttctggacttgtcggtggatactatgctccttctctctttattggcggggcagcaggaatggcctatggaaagtttattggacttgctttggctcagaaccctgatttcaatctctctatcttggaagtggcatctccacaagcttatggtctggttggaatggctgctacacttgcgggggtttgtcaagttcctcttacagcagtactactgctatttgaacttacacaggattatcgtatagtgttacctctactgggagctgtaggcatgtcttcatggattacatctggacaatcaaagagacaagaaactagagaaacaaaagaaactaggaaaagaaagagccaagaagctgtacagtctctgacgtcatctgatgatgaatcatcaacgaataacctttgtgaagttgaaagttctctttgccttgatgattctctcaaccaatctgaggagctgccgaagagtatttttgtttcagaagccatgcgaacaagatttgcgacagttatgatgagcacttctttggaagaggcattaactcgtatgctgatagagaaacaatcctgcgccttgattgttgatcctgacaatatctttctcggtatacttacactttcagacattcaggaattcagcaaagcaagaaaagaaggaaataatagacccaaggatatttttgttaatgacatctgttcgaggagtggaggaaaatgtaaagtgccatggactgttacacctgatatggatcttctcgctgcccaaacaatcatgaacaagcatgaactttctcatgttgcagtcgtttcaggcagcattgatgctcccagaatacaccctgttggggtcctggatagagaatgtatcactctaacacgcagggctctagcaaccagaatgtacctcctaaattcgctgtatctgtaa

SEQ ID NO: 38 - DNA, 750 bp, Arabidopsis thaliana
atggcgtcggcgtcctcgtctgacggagttgccggaaggattcagaacgcttctttggttcttgtctccgataacagttccacgcttgctgatatccgcaaagctgtggcaatgatgaagaacattgcagttcaattggagaaagaaaatcaaacggacaaggttaaggaccttgaaaattctgtggctgagttattggatttgcatagtgattgtaatcaccgttcgacagcaattcaatccgttgcaaatcggtaccaacccgtggaacaattaacggactttaaaaagttgcttgatgatgaattcacaaagctcaaggctacaccttcctcagtgccacaaaatgatcatttgatgcgccagttcagggaagcagtttggaatgttcatcatgcaggtgaaccaatgcctggtgacgatgatgaggacattgttatgaccagtactcagtgccctcttctaaacatgacatgtcccttgagcgggaagcctgtcactgaattagcagatccagttcgcagtatggattgcaggcacgtctatgaaaaatctgtaatcctgcattacatagtcaacaatccaaatgcgaattgtcctgtagcagggtgccgaggtaaactgcagaatagcaaagtgatttgtgatgcaatgttgaagtttgaaatagaggagatgcgctcgttgaacaaacaatctaatagggctgaagtgattgaagacttcacagaagatgtggatgaagattag

SEQ ID NO: 39 - DNA, 846 bp, Arabidopsis thaliana
atgtcaacctccgccgcttccttgtgttgttcatcaacccaggtcaatgggtttggtcttaggcctgaaaggtcgcttctttaccaacccacttccttttctttctccagaaggagaactcatggaattgtcaaggcctcatctcgggttgataggttttcgaaaagtgatatcattgtttctccctctattctctcggctaatttcgccaaattaggcgagcaggtaaaagcagtggagttggcaggttgtgattggattcatgttgatgtcatggacggtcgttttgttcccaacattactatcggacctctcgtggttgatgctttgcgccctgtgacagatcttcctttggatgttcatctgatgatagtggaacccgagcagagagtaccggatttcatcaaagcaggtgcagatattgtcagtgtacattgtgaacagcaatccaccatccatttgcatcgtaccgtcaatcaaataaaaagcttaggggctaaagctggagttgttctaaaccctggaaccccattgagtgcaatagaatatgtcttggatatggtggatctggtcttgatcatgtcggtcaaccctggttttggtggacagagctttattgaaagccaagtaaagaaaatctcggacttgaggaaaatgtgtgcagagaagggagtaaacccatggattgaagttgatggtggtgtcactccagcgaatgcgtacaaggttattgaggctggagcaaatgctctagtggctggatcagctgtatttggagctaaggactacgcagaagctataaaaggaattaaggccagcaaacgaccagcagctgtagctgtgtaa

SEQ ID NO: 40 - DNA, 1482 bp, Arabidopsis thaliana
atggttttgtctaagacagtttccgaatctgatgtctcaatccattcaacttttgcttctcgttacgtccgcaactctcttccacgattcgaaatgcctgagaactcaatcccaaaagaagcagcttaccaaatcatcaacgacgagctaatgctcgatggtaacccaaggctgaacctagcttccttcgtgaccacatggatggagccagaatgtgacaagctcatgatggagtccatcaacaagaactacgtcgacatggacgagtaccctgtcaccactgagcttcagaaccgatgtgttaacatgatagcacgtctcttcaacgcgccgcttggtgacggtgaagctgccgttggtgttggcaccgtcggatcgtcggaggcgattatgttggccggtttggcttttaagagacaatggcagaataagcgtaaggcccaagggcttccttatgataagcccaatatcgtaaccggtgctaatgtccaggtttgctgggagaaattcgcaaggtatttcgaagtggagcttaaggaagtgaacctaagagaagactattacgtgatggaccctgtaaaggcggtcgaaatggtagacgaaaacacaatttgtgtcgctgccatcctcggttcaacgttaaccggtgaattcgaagacgttaagctcctcaacgacctccttgtcgagaaaaacaagcaaaccggatgggacacgccaatacacgtggacgcagcgagtggtgggtttattgctccgttcttgtatccggagctggagtgggatttccggctaccgttggttaagagtattaatgtgagtggtcacaaatacggtttggtttacgccggtattggttgggttgtatggagaaccaaaaccgatttgcctgatgaacttatcttccatatcaattatcttggcgctgatcaaccaacctttacactcaacttctccaaaggttcaagtcaagtgattgctcagtactaccagctgattcgtcttggattcgagggttatcgcaatgtgatggataattgtcgggaaaacatgatggtactaagacaaggattagagaaaacgggacgttttaaaatcgtctccaaagaaaacggtgttccgttagtggcgttttctctcaaagatagtagccgccacaacgagttcgaggtggcccatacactccgtcgcttcggctggatcgttccggcctacacgatgcctgcggatgcgcagcatgtcactgtccttcgagttgttatccgagaagatttctctcgaaccttagccgagagattggtagctgatttcgagaaggttctacacgagctcgatacgcttccggcgagggttcacgccaagatggctaatggaaaagttaacggtgttaagaagacgccagaggagacgcagagagaagtcacggcctactggaagaagttgttggagactaagaagaccaacaagaacacaatttgctaa

SEQ ID NO: 41 - DNA, 351 bp, Zea mays
atgactgaaacaagagagatcaacgttttcatggccaagctcgctgagcaggctgaacgttacgatgagatggttgaagccatgaagaacgttgctgatttgggacaagaactcaccgttgaagagcgtaaccttctctccgttgcctacaagaacgtcattggtgcccgtagagcttcatggagaatcatcacctctattgagcaaaaggaagaatccaagggaaacaccgctcacgttgagagaatcaaggagtacagaaagaaggtcgagaacgaagtctccaagatctgcgctgatgtcctcggaaccctcgacaacaagttgattccaaacgctcaaaccacctag

SEQ ID NO: 42 - DNA, 1554 bp, Arabidopsis thaliana
atgtctccggaagcttacgttctgttctttaacagttttaacctcgtaaccttcgaagcctttgcttcagtctcacttatcatagccacagttgctttcttgctctcaccaggtgggctcgcatgggcctggaccgggtcatccaagagtcgggtttcgattccaggaccatctggttctctttccgtcttctccggctccaatccccaccgtgttctcgccgctcttgctaaacgcttcaaggcctctccgttgatggcgttctcagttgggttttcgcgtttcgttatctctagtgaaccggagacggctaaagagattttgagcagctctgcttttgctgaccggccggttaaggagtcagcttacgagcttttgtttcaccgtgccatgggattcgcaccgtatggtgagtattggaggaatctgaggagaatctcctccactcatcttttcagtccaagaagaatcgcgagttttgagggtgttagagttggcatcggtatgaagatggtcaagaagattaaaagccttgttacgtctgatgcttgtggtgaagttgaagtgaaaaagatcgttcactttggttctttgaataatgtaatgacgacagtgtttggtgaaagctacgattttgatgaagttaatggaaaagggtgttttttggagaggctggtgagtgaaggctacgagttgcttgggatttttaactggagtgatcacttttggtttcttcgttggtttgacttccaaggagtgaggaagaggtgtagagctttggtctctgaagtcaacacttttgtcggcggaataattgagaaacacaagatgaagaagggtaataatctcaatggagaggaaaatgacttcgttgatgtcttgcttggcttgcaaaaggatgaaaagttgtctgattctgacatgattgctgttctttgggaaatgatatttagagggacagatacagttgcgattctagtggaatgggtgcttgcaagaatggttttgcatcaagacatccaagataaactctacagagagatagcttctgctacaagtaacaatattagatccttgtctgattccgacatcccaaaactgccgtaccttcaagctattgtcaaagaaaccctaaggctccacccccctggtccacttctctcttgggctcgtctcgctatccatgacgtccacgtaggtcctaaccttgtccctgctggaaccatagctatggtcaacatgtggtccatcacacacaacgctaaaatctggaccgaccctgaagcgtttatgcctgaaaggttcattagtgaggatgtgagcatcatgggctcggatcttagattggctccattcggatccggtcgtcgggtttgtcccggtaaagcaatgggtctagctactgttcatctctggattggtcaactaattcagaattttgaatgggtgaagggttcttgtgatgttgagctcgctgaggttctgaagctgtctatggagatgaagaatccgttgaagtgcaaggctgttccaaggaatgttggtttcgcttag

SEQ ID NO: 43 - DNA, 387 bp, Zea mays
atggcggcctccatgatctcctcgtcagctctggcggtggcgcctcagggcctgccgcccctcggccgccgcgcctcctccttcgccgtcgtctgctccaagaagaagatcaagaccgacaagccctacgggattgggggtggcctgaccgtcgacgtcgacgccaacgggagaaagggcaagggcaagggcgtgtaccagttcgtcgacaagtacggcgcgaacgtcgacggatacagcccaatctacaacgaggatgactggtctcccaccggcgacgtctacgtcggtggaaccactgggcttctgatctgggccgtcaccctcgctgggatcctcggcggcggcgccctcctcgtctacaacaccagcgccctctccggctaa

SEQ ID NO: 44 - DNA, 237 bp, Zea mays
atgggcggtctctccaccaagcttttcgtggtcctcctcctgctcgtttgttacaccgggacgcaaggcgggccggtgactatggtgtcggcgaggaagtgcgagtcgcagagcttccgcttcaagggaccttgctcgagggacgccaactgcgcaaacgtctgcctgaccgaaggtttcaccggcggcgtgtgcaagggcctacgccaccgctgcttctgcaccagggactgctag

SEQ ID NO: 45 - protein, 635 aa, Lycopersicon esculentum
MEVCNCIEPQWPADELLMKYQYISDFFIAIAYFSIPLELIYFVKKSAVFPYRWVLVQFGAFIVLYGATHLINLWTFTTHSRTVALVMTTAKVLTAAVSCITALMLVHIIPDLLSVKTRELFLKTRAEELDKEMGLIIRQEETGRHVRMLTHEIRSTLDRHTILKTTLVELGRTLDLAECALWMPCQGGLTLQLSHNLNNLIPLGSTVPINLPIINEIFSSPEAIQIPHTNPLARMRNTVGRYIPPEVVAVRVPLLHLSNFTNDWAELSTRSYAVMVLVLPMNGLRKWREHELELVQVVADQVAVALSHAAILEDSMRAHDQLMEQNIALDVARQEAEMAIRARNDFLAVMNHEMRTPMHAVIALCSLLLETDLTPEQRVMIETILKSSNLLATLINDVLDLSRLEDGILELENGTFNLHGILREAVNLIKPIASLKKLSITLALALDLPILAVGDAKRLIQTLLNVAGNAVKFTKEGHISIEASVAKPEYARDCHPPEMFPMPSDGQFYLRVQVRDTGCGISPQDIPLVFTKFAESRPTSNRSTGGEGLGLAICRRFIQLMKGNIWIESEGPGKGTTVTFVVKLGICHHPNALPLLPMPPRGRLNKGSDDLFRYRQFRGDDGGMSVNAQRYQRSM

SEQ ID NO: 46 - protein, 133 aa, Oryza sativa
MGMAQSSSSSSRPSDSEQLEEPSKPVMALDKAKEIVASSPIVVFSKTYCPFCARVKRLLAELAASYKAVELDVESDGSELQSALADWTGQRTVPCVFIKGKHIGGCDDTMAMHKGGNLVPLLTEAGAIATPSL

SEQ ID NO: 47 - protein, 238 aa, Oryza sativa
MGEEAPEEYELGGGEDERVMEWETGLPGADELTPLSQPLVPAGLAAAFRIPPEPGRTLLDVHRASAATVSRLRRASSSSSSSFPAFASKGAGTGADEAESGGGADGGNGNTNNSSSKRARLVWTPQLHKRFVEVVAHLGMKNAVPKTIMQLMNVEGLTRENVASHLQKYRLYVKRMQGLSNEGPSPSDHIFASTPVPHASLHDQVPSPYHPHPHHHSYNNAAYAATVSSYHHYHHANH

SEQ ID NO: 48 - protein, 252 aa, Capsicum annuum
MNQDMALEQLDTTFNKHDTPLGKWKSMNDEVEENISGGFDCNICLDCVHEPVITLCGHLYCWPCIYKWIYFQSVSSENSDQQQPQCPVCKAEVSEKTLIPLYGRGGQSTKPSEGKAPNLGIVIPQRPPSPRCGGHFLLPTTDSNPSQLLQRRGYQQQSQTRQPAYQGSYMSSPMLSPGGATANMLQHSMIGEVAYARIFGNSSTTMYTYPNSYNLAISSSPRMRRQLSQADRSLGRICFFLFCCFVTCLILF

SEQ ID NO: 49 - protein, 313 aa, Arabidopsis thaliana
MDEGVIAVSAMDAFEKLEKVGEGTYGKVYRAREKATGKIVALKKTRLHEDEEGVPSTTLREISILRMLARDPHVVRLMDVKQGLSKEGKTVLYLVFEYMDTDVKKFIRSFRSTGKNIPTQTIKSLMYQLCKGMAFCHGHGILHRDLKPHNLLMDPKTMRLKIADLGLARAFTLPMKKYTHEILTLWYRAPEVLLGATHYSTAVDMWSVGCIFAELVTNQAIFQGDSELQQLLHIFKLFGTPNEEMWPGVSTLKNWHEYPQWKPSTLSSAVPNLDEAGVDLLSKMLQYEPAKRISAKMAMEHPYFDDLPEKSSL

SEQ ID NO: 50 - protein, 985 aa, Arabidopsis thaliana
MFLCFCPCHVPIMSRLSPATGISSRLRFSIGLSSDGRLIPFGFRFRRNDVPFKRRLRFVIRAQLSEAFSPDLGLDSQAVKSRDTSNLPWIGPVPGDIAEVEAYCRIFRSAERLHVALMETLCNPVTGECRVPYDFSPEEKPLLEDKIVSVLGCILSLLNKGRKEILSGRSSSMNSFNLDDVGVAEESLPPLAVFRGEMKRCCESLHIALENYLTPDDERSGIVWRKLQKLKNVCYDAGFPRSDNYPCQTLFANWDPIYSSNTKEDIDSYESEIAFWRGGQVTQEGLKWLIENGFKTIVDLRAEIVKDTFYQTALDDAISLGKITVVQIPIDVRMAPKAEQVELFASIVSDSSKRPIYVHSKEGVWRTSAMVSRWKQYMTRPITKEIPVSEESKRREVSETKLGSNAVVSGKGVPDEQTDKVSEINEVDSRSASSQSKESGRFEGDTSASEFNMVSDPLKSQVPPGNIFSRKEMSKFLKSKSIAPAGYLTNPSKILGTVPTPQFSYTGVTNGNQIVDKDSIRRLAETGNSNGTLLPTSSQSLDFGNGKFSNGNVHASDNTNKSISDNRGNGFSAAPIAVPPSDNLSRAVGSHSVRESQTQRNNSGSSSDSSDDEAGAIEGNMCASATGVVRVQSRKKAEMFLVRTDGVSCTREKVTESSLAFTHPSTQQQMLLWKTTPKTVLLLKKLGQELMEEAKEAASFLYHQENMNVLVEPEVHDVFARIPGFGFVQTFYIQDTSDLHERVDFVACLGGDGVILHASNLFKGAVPPVVSFNLGSLGFLTSHPFEDFRQDLKRVIHGNNTLDGVYITLRMRLRCEIYRKGKAMPGKVFDVLNEIVVDRGSNPYLSKIECYEHDRLITKVQGDGVIVATPTGSTAYSTAAGGSMVHPNVPCMLFTPICPHSLSFRPVILPDSAKLELKIPDDARSNAWVSFDGKRRQQLSRGDSVRIYMSQHPLPTVNKSDQTGDWFRSLIRCLNWNERLDQKAL

SEQ ID NO: 51 - protein, 378 aa, Oryza sativa
MATAVASQVAVSAPAGSDRGLRSSGIQGSNNISFSNKSWVGTTLAWESKATRPRHANKVLCMSVQQASESKVAVKPLDLESANEPPLNTYKPKEPYTATIVSVERIVGPKAPGETCHIVIDHGGNVPYWEGQSYGIIPPGENPKKPGAPHNVRLYSIASTRYGDSFDGRTTSLCVRRAVYYDPETGKEDPSKNGVCSNFLCNSKPGDKVKVTGPSGKIMLLPEEDPNATHIMIATGTGVAPFRGYLRRMFMEDVPKYRFGGLAWLFLGVANTDSLLYDEEFTSYLKQYPDNFRYDKALSREQKNKNAGKMYVQDKIEEYSDEIFKLLDGGAHIYFCGLKGMMPGIQDTLKKVAEQRGESWEQKLSQLKKNKQWHVEVY

SEQ ID NO: 52 - protein, 559 aa, Cyanidium caldarium
MCGILAVLGSSLPVEELRELVKSCTKKLYHRGPDEEQYFISEDGWCGLGFARLKIVDPEHGVQPMFNDQRTVWSVTNGELYNHEEIRKTELNNMTLHSHSDCEIMIPLYEKYVSSQRYDHDIQYVYNLLRGVFASCLVDLKRGFFMAGRDPIGVRALFYGTSKDGAVWFASEAKAIVDVCDYVTAFIPGTFVKGYRGREQAFSFTRYYEPVYWHDHWMPVSPVDYQLLHDTFVLSCKRRLMSDVPIGVFISGGLGSSLVASVAKRLLDPNYDFHSFACGLEGAPDVAAAQRVADFLGTKHHVLTFTVEEGIQALDQVIYHLETYDVTTVRASTPMYLLSGLCKKYVKVVLSGEGADEIFGGYLYFHNAPNEIAFHQEVVRRVKLLYTADVLRGDRATAAQSLELRVPFLDRDFLDVAMSIHPREKVTSKHRIEKYIIRYAFSKEFCGEEYLPDDILWRQKEQFSDGVGYSWIDGLKAYCEKAVSDADLQNAAQRFPHDTPTTKEAYVYRAIFEKHFGNCKAVQGLRESVARWVPMWSDSTDPSGRAQKVHVAAYSNGGD

SEQ ID NO: 53 - protein, 516 aa, Arabidopsis thaliana
MGLGGDQSFVPVMDSGQVRLKELGYKQELKRDLSVFSNFAISFSIISVLTGITTTYNTGLRFGGTVTLVYGWFLAGSFTMCVGLSMAEICSSYPTSGGLYYWSAMLAGPRWAPLASWMTGWFNIVGQWAVTASVDFSLAQLIQVIVLLSTGGRNGGGYKGSDFVVIGIHGGILFIHALLNSLPISVLSFIGQLAALWNLLGVLVLMILIPLVSTERATTKFVFTNFNTDNGLGITSYAYIFVLGLLMSQYTITGYDASAHMTEETVDADKNGPRGIISAIGISILFGWGYILGISYAVTDIPSLLSETNNSGGYAIAEIFYLAFKNRFGSGTGGIVCLGVVAVAVFFCGMSSVTSNSRMAYAFSRDGAMPMSPLWHKVNSREVPINAVWLSALISFCMALTSLGSIVAFQAMVSIATIGLYIAYAIPIILRVTLARNTFVPGPFSLGKYGMVVGWVAVLWVVTISVLFSLPVAYPITAETLNYTPVAVAGLVAITLSYWLFSARHWFTGPISNILS

SEQ ID NO: 54 - protein, 453 aa, Arabidopsis thaliana
MASLMLSLGSTSLLPREINKDKLKLGTSASNPFLKAKSFSRVTMTVAVKPSRFEGITMAPPDPILGVSEAFKADTNGMKLNLGVGAYRTEELQPYVLNVVKKAENLMLERGDNKEYLPIEGLAAFNKATAELLFGAGHPVIKEQRVATIQGLSGTGSLRLAAALIERYFPGAKVVISSPTWGNHKNIFNDAKVPWSEYRYYDPKTIGLDFEGMIADIKEAPEGSFILLHGCAHNPTGIDPTPEQWVKIADVIQEKNHIPFFDVAYQGFASGSLDEDAASVRLFAERGMEFFVAQSYSKNLGLYAERIGAINVVCSSADAATRVKSQLKRIARPMYSNPPVHGARIVANVVGDVTMFSEWKAEMEMMAGRIKTVRQELYDSLVSKDKSGKDWSFILKQIGMFSFTGLNKAQSDNMTDKWHVYMTKDGRISLAGLSLAKCEYLADAIIDSYHNVS

SEQ ID NO: 55 - protein, 555 aa, Oryza sativa
MATTATLPFSCSSTLQTLTRTIPLRLRLHRRRFLHHLPSLAALPRLPLPRPPLLPHARRHVSASAAPNGASSEGEYDYDLFTIGAGSGGVRASRFASTLYGARAAVCEMPFATVASDDLGGVGGTCVLRGCVPKKLLVYGSKYSHEFEESHGFGWVYETDPKHDWNTLIANKNTELQRLVGIYKNILNNSGVTLIEGRGKIVDPHTVSVDGKLYTARNILIAVGGRPSMPNIPGIEHVIDSDAALDLPSKPEKIAIVGGGYIALEFAGIFNGLKSEVHVFIRQKKVLRGFDEEVRDFIAEQMSLRGITFHTEQSPQAITKSNDGLLSLKTNKETIGGFSHVMFATGRKPNTKNLGLEEVGVKLDKNGAIMVDEYSRTSVDSIWAVGDVTDRVNLTPVALMEGGAFAKTVFGDEPTKPDYRAVPSAVFSQPPIGQVGLTEEQAIEEYGDVDIYTANFRPLRATLSGLPDRIFMKLIVCATTNKVVGVHMCGEDAPEIIQGVAIAVKAGLTKQDFDATIGIHPTSAEEFVTMRNATRKVRRSTTDEVESKDKVVTQN

SEQ ID NO: 56 - protein, 496 aa, Oryza sativa
MARKMLKDEEVEVAVTDGGSYDYDLFVIGAGSGGVRGSRTSASFGAKVAICELPFHPISSDWQGGHGGTCVIRGCVPKKILVYGSSFRGEFEDAKNFGWEINGDINFNWKRLLENKTQEIVRLNGVYQRILGNSGVTMIEGAGSLVDAHTVEVTKPDGSKQRYTAKHILIATGSRAQRVNIPGKELAITSDEALSLEELPKRAVILGGGYIAVEFASIWKGMGAHVDLFYRKELPLRGFDDEMRTVVASNLEGRGIRLHPGTNLSELSKTADGIKVVTDKGEEIIADVVLFATGRTPNSQRLNLEAAGVEVDNIGAIKVDDYSRTSVPNIWAVGDVTNRINLTPVALMEATCFSKTVFGGQPTKPDYRDVPCAVFSIPPLSVVGLSEQQALEEAKSDVLVYTSSFNPMKNSISKRQEKTVMKLVVDSETDKVLGASMCGPDAPEIIQGMAVALKCGATKATFDSTVGIHPSAAEEFVTMRTLTRRVSPSSKPKTNL

SEQ ID NO: 57 - protein, 475 aa, Agrobacterium tumefaciens
MERLAKLPVFWGLEGKRVVLTGGSDGAAWKAELLLACGAQLDLYCEESGLSESLATLVAKSPMLTWHDRCWDADIFKGAELALADCEAEEEAGRFYHAARAAGVPVNVIDKPEFCQFQFGSIVNRSPVVVSISTDGAAPILAQAIRRRIETLLPLSLKDWGALAQTIRERVNLRLAPGAARRSFWEKFVDRAFTERLDEGSEERLLKDVATRTGLAESGRGFVTLVGAGPGDAELLTLKAVRALQAADVILFDDLVSAEVLELARREAKRMLVGKRGGRESCKQEDINDMMIRFAKAGRRVVRLKSGDPMIFGRAGEEIAALEAENIPVEVVPGITAASAMASRLGVSLTHRDHAQSVRFVTGHSRQGKLPENIDWQSLSNPSVTTVFYMGGRTAADIQSCLLAHGMPASTPVVVMISVSRVNEQRWCGSLAQLVAAVERLGVNEPVLIGVGDAFRSASVNGGEQTAAAPFQKAG

SEQ ID NO: 58 - protein, 356 aa, Zea mays
MACLTDLVNLNLSDNTEKIIAEYIWIGGSGMDLRSKARTLSGPVTDPSKLPKWNYDGSSTGQAPGEDSEVILYPQAIFKDPFRRGNNILVMCDCYTPAGEPIPTNKRYNAAKIFSSPEVAAEEPWYGIEQEYTLLQKDTNWPLGWPIGGFPGPQGPYYCGIGAEKSFGRDIVDAHYKACLYAGINISGINGEVMPGQWEFQVGPSVGISSGDQVWVARYILERITEIAGVVVTFDPKPIPGDWNGAGAHTNYSTESMRKEGGYEVIKAAIEKLKLRHREHIAAYGEGNERRLTGRHETADINTFSWGVANRGASVRVGRETEQNGKGYFEDRRPASNMDPYVVTSMIAETTIIWKP

SEQ ID NO: 59 - protein, 343 aa, Arabidopsis thaliana
MFGRGPSKKSDNTKFYEILGVPKSASPEDLKKAYKKAAIKNHPDKGGDPEKFKELAQAYEVLSDPEKREIYDQYGEDALKEGMGGGGGGHDPFDIFSSFFGGGPFGGNTSRQRRQRRGEDVVHPLKVSLEDVYLGTMKKLSLSRNALCSKCNGKGSKSGASLKCGGCQGSGMKVSIRQLGPGMIQQMQHACNECKGTGETINDRDRCPQCKGDKVIPEKKVLEVNVEKGMQHSQKITFEGQADEAPDTVTGDIVFVLQQKEHPKFKRKGEDLFVEHTLSLTEALCGFQFVLTHLDGRSLLIKSNPGEVVKPDSYKAISDEGMPIYQRPFMKDEEEGTSSKRGL

SEQ ID NO: 60 - protein, 574 aa, Zea mays
MAADPSSSSTGQQTADIRAAPPEDSRQMAMSGPLNVRGDRRPPPMQRAFSRQVSLGSGVTVLGMDRAGRSGGARGQRTLPRSGRSLGVLNHSGGLVQAAGDGAARRVGDFSMFRTKSTLSKQNSMLPTRIRESDLELPTHVEDPQSASSRPAEDPLNKSVPAGRYFAALRGPELDEVRDTEDILLPKDEVWPFLLRFPIGCFGVCLGLGSQAILWGALAASPAMRFLHVTPMINVALWLLAVAVLVATSVTYALKCIFYFEAIRREYFHPVRVNFFFAPWIAAMFVTIGLPRSYAPERPHPAVWCAFVLPLFALELKIYGQWLSGGKRRLCKVANPSSHLSVVGNFVGAILAARVGWTEAGKLLWAIGVAHYIVVFVTLYQRLPTNEALPKELHPVYSMFIATPSAASLAWAAIYGSFDAVARTFFFMAIFLYLSLVVRINFFRGFRFSLAWWSYTFPMTTASLATVKYAEAVPCFASRALALSLSLMSSTMVSMLLVSTLLHALVWRSLFPNDLAIAITKDRQNGAVKPNDRGKRASKRVHDIKRWAKQAPLSLVSSITKSHSADKEEEERTE

SEQ ID NO: 61 - protein, 245 aa, Zea mays
MAQEDVHLDDAGLALCLSLHGTSSSRLSTEAPRTLEPPSLTLSMPDEATATATGGSGGSGGAARSVSSRSVEGVKRERVDDAEGERASSTAAAARVCAGAEDDDDGSTRKKLRLTKEQSKLLEDRFKDHSTLNPKQKIALAKQLKLRPRQVEVWFQNRRARTKLKQTEVDCELLKRCCESLSEENRRLQRELQELRALKLAGPHPQAPSSSPAAATQGVPVPVPPPLYVQMQMQLSSCRCCRPPR

SEQ ID NO: 62 - protein, 247 aa, Zea mays
MEKEEGFGKSWLGLGIGGGGRDLNLMKRSRPLRPVRLDLLFPPSVEGGEAAARSRKAGAGALRNMSLKQVAGDDDGGQSSHGGPSPSDDDDGAGARKKLRLTTEQSKLLEDTFRAHNILSHAQKHEVARQVDLSARQVEVWFQNRRARTKLKQTEVDCETLRRWRESLADENLRLRLELEQLQRWATAAAGQSSASPSPATATASVCPSCDKVVVVTVTSCGETSGKSSTSSYSSSPPLDMLDRSVQ

SEQ ID NO: 63 - protein, 295 aa, Zea mays
MMPQASASLDLGLSLGLTLTSQGSLSSSTTTAGSSSPWAAALSSVVADVARARGDAYAQHHAGAAMTMRASTSPDSGDTTTAKREREGELERTGSAGGVRSDEEDGADGGAGGRKKLRLSKDQAAVLEECFKTHSTLNPKQKVQLANRLGLRPRQVEVWFQNRRARTKLKQTEVDCEYLKRWCDRLADENKRLEKELADLRALKAAPPSSAAAQPASAAATLTMCPSCRRVAAAASHHHQPPPPQCHPKPTVAAGGGSVVPRPSHCQFFPAAAVDRTSQGTWNTAAPPLVTRELF

SEQ ID NO: 64 - protein, 195 aa, Oryza sativa
MGEEAVVMEAPRPKSPPRYPDLCGRRRMQLEVQILSREITFLKDELHFLEGAQPVSRSGCIKEINEFVGTKHDPLIPTKRRRHRSCRLFRWIGSKLCICISCLCYCCKCSPKCKRPRCLNCSCSSCCDEPCCKPNCSACCAGSCCSPDCCSCCKPNCSCCKTPSCCKPNCSCSCPSCSSCCDTSCCKPSCTCFNI

SEQ ID NO: 65 - protein, 357 aa, Oryza sativa
MQKQHAADSAALVAAMGEVHRLRVQLAAAARADRKQDVVEAMATIDELRVKLKASEEAEAQARALHEECKQQLETSRATIDSLLTDGSKLMDSFSLVVKELEESRAKVKALEEEIAETSAAKAGERCNCSASASASEVAELRSELESTEARFQEERILSTVETQCAYELMDQIKMESDSRHGKLAAALESTKSEVIFLKASLFDKDSELRRALDANEKLQSETRTDNELKEQLQGALLENGQLKRELQQHTSEKKASAKATDAADAAAEAAKKGEMEAELRRLRVQAEQWRKAAETAMALLTVGKGGNGKVVDRSESLEGGGGGGGKYAGLWDELDDDAAARKNGNVLRRISGMWKK

SEQ ID NO: 66 - protein, 158 aa, Arabidopsis thaliana
MGEIGFTEKQEALVKESWEILKQDIPKYSLHFFSQILEIAPAAKGLFSFLRDSDEVPHNNPKLKAHAVKVFKMTCETAIQLREEGKVVVADTTLQYLGSIHLKSGVIDPHFEVVKEALLRTLKEGLGEKYNEEVEGAWSQAYDHLALAIKTEMKQEES

SEQ ID NO: 67 - protein, 249 aa, Solanum lycopersicum
MAGGVAIGSFSDSFSVVSLKSYLAEFISTLIFVFAGVGSAIAYGKLTTNAALDPAGLVAIAVCHGFALFVAVSISANISGGHVNPAVTCGLTFGGHITFITGSFYMLAQLTGAAVACFLLKFVTGGCAIPTHGVGAGVSILEGLVMEIIITFGLVYTVFATAADPKKGSLGTIAPIAIGLIVGANILAAGPFSGGSMNPARSFGPAMVSGNFEGFWIYWIGPLVGGSLAGLIYTNVFMTQEHAPLSNEF

SEQ ID NO: 68 - protein, 406 aa, Arabidopsis thaliana
MSLSPRVQSLKPSKTMVITDLAATLVQSGVPVIRLAAGEPDFDTPKVVAEAGINAIREGFTRYTLNAGITELREAICRKLKEENGLSYAPDQILVSNGAKQSLLQAVLAVCSPGDEVIIPAPYWVSYTEQARLADATPVVIPTKISNNFLLDPKDLESKLTEKSRLLILCSPSNPTGSVYPKSLLEEIARIIAKHPRLLVLSDEIYEHIIYAPATHTSFASLPDMYERTLTVNGFSKAFAMTGWRLGYLAGPKHIVAACSKLQGQVSSGASSIAQKAGVAALGLGKAGGETVAEMVKAYRERRDFLVKSLGDIKGVKISEPQGAFYLFIDFSAYYGSEAEGFGLINDSSSLALYFLDKFQVAMVPGDAFGDDSCIRISYATSLDVLQAAVEKIRKALEPLRATVSV

SEQ ID NO: 69 - protein, 282 aa, Gossypium raimondii
GEKKLATIYNVVAVIRGLEEPDRYVLMGNHRDAWTYGAVDPNSGTATLLDIARRYALLMRKGWNPRRTIIFCSWDAEEFGMIGSTEWVEQNLVNLGAKAVAYLNVDCAVQGPGFFAGATPQLDNLIFEVTKKVQDQDSEVVATIYEKWKTMNGNNIQRLSGVDSDFAPFLQHAGVPSVDIYYGRDFPVYHTAFDSFNWMINNADPFFWRHVAVAGVWGLLGLHLADDPVLPLDYLSYAKQLQVWGYSLLVFVDIVKCSQPFPLLLFFYKVLVGLLIVNPWLQ

SEQ ID NO: 70 - protein, 354 aa, Zea mays
MELGLSLGDAAVPDAGRAAPELGLGLGVGIGSNAAGTGRGSKAAGTTGTTGWWAAPATPESAVRLSLVSSLGLQWPPPDGGICHVGRDEAPARGFDVNRAPSVAGSALALEDDEEEPGAAALSSSPNDSAGSFPLDLGGPRAHAEGAAARAGGERSSSRASDEDEGASARKKLRLSKEQSAFLEESFKEHSTLNPKQKAALAKQLNLRPRQVEVWFQNRRARTKLKQTEVDCEYLKRCCETLTEENRRLHKELAELRALKTAPPFFMRLPATTLSMCPSCERVASGPSPASTSAPASSTPPATAATTAISYAAAAAAPVRADHRPSSFAALFAATRSFPLASQPRPPAPASNCL

SEQ ID NO: 71 - protein, 293 aa, Thellungiella halophila
MLKVPEHQVAGHIAIDGKLGPLVDDQGRFFKPLQDDARGENEAKFYESFSANKNVPDHIHRYFPVYHGTQLVEASDGSGKLPHMVLEDVVSEYSNPSIMDVKIGSRTWYPDVSEEYFKKCIKKDRETTTVSLGFRVSGFKIFDHQESSFWRPEKKVVLGYKVDGARLALKKFVSSNSPVESKSMPNCAFASEVYGGPNGILAQLLELKAWFETQTIYHFNSCSILMVYENDSMLMKGGDDAQMPRAQVKLVDFAHVLDGNGVIDHNFLGGVCSFIKFIQDILETDTSQLENGH

SEQ ID NO: 72 - protein, 522 aa, Sesbania rostrata
MGYETRRLSDEYEVSDVLGRGGFSVVRKGTKKSSSEKTLVAIKTLRRLGASNNNPSGLPKTKGGEKSIATMMGFPTWRQVSVSDALLTNEILVMRRIVENVSPHPNVIDLYDVYEDSNGVHLVLELCSGGELFDRIVAQDRYSETEAAAVVRQIAAGLEAIHKANIVHRDLKPENCLFLDTRKDSPLKIMDFGLSSVEEFTDPVVGLFGSIDYVSPEALSQGKITTKSDMWSLGVILYILLSGYPPFIAPSNRQKQQMIVNGNFSFYEKTWKGISQSAKQLISSLLTVDPSKRPSAQQLLSHPWVIGEKAKDDQMDPEIVSRLQSFNARRKLRAAAIASVWSSTVFLRTKKLRSLVGTHDLKEEEIENLRIHFKKICANGDNATLSEFEEVLKAMNMPSLIPLAPRIFDLFDNNRDGTVDMREILCGFSSLKNSKGDDALRLCFQMYDTDRSGCITKEEVASMLRALPDDCLPADITEPGKLDEIFDLMDANSDGKVTFDEFKAAMQRDSSLQDVVLSSLRP

SEQ ID NO: 73 - protein, 91 aa, Arabidopsis thaliana
MIKLLFTYICTYTYKLYALYHMDYACVCMYKYKGIVTLQVCLFYIKLRVFLSNFTFSSSILALKNPNNSLIKIMAILPENSSNLDLTISVP

SEQ ID NO: 74 - protein, 561 aa, Zea mays
MLSEDFIVADIAIHPRHARIMKPHQLEGFNFLVKNLIGDKPGGCILAHAPGTGKTFMLISFIQSFMARYPSARPLVVLPKGILGIWKTEVKRWQVQDIPVYDFYSVKAEKRVEQLQILKSWEDKMSILFLGYKQFSTIVTDDGGSNVTAACRDRLLKVPNLLILDEGHTPRNRETNVLESLNRVETPRKVVLSGTLFQNHVEEVFNILNLVRPKFLRMESSRPTARRIMSQVEIVGRSSKGLADGAFTKAVEETLLNDENFKRKAHVIRGLRELTKDVLHYYKGDILDELPGLVDFSVFLKLTPKQKDIIYKLEAHDRFKRNAVGSALYIHPCLSELSEVNAEHRANTFRDDLVDSLVDSITVRDGVKANFFMNILSLANSAGEKVLAFSQYISPMIFFERLLVKKKGWHVGKEIFMISGDTSQEDRELATDHFNNSADAKIMFGSIKACGEGISLVGASRVVILDVHLNPSVTRQAIGRAFRPGQQKKVFVYRLVAADSDEVKVHETAFKKEVIPKLWFEWSEHCTTEDFKLGQIDIDDSGDELLDTKAIRKDIKALYRR

SEQ ID NO: 75 - protein, 290 aa, Arabidopsis thaliana
MVVTALWCGILISSQQLSFHVTSSIAISQVLFVSSILIWVSYESSAIKGFRKIDPDRWEFANEGFLAGQKHLLKNIKRRRNMGLQNVNQQGSGMSCVEVGQYGFDGEVERLKRDHGVLVAEVVRLRQQQHSSKSQVAAMEQRLLVTEKRQQQMMTFLAKALNNPNFVQQFAVMSKEKKSLFGLDVGRKRRLTSTPSLGTMEENLLHDQEFDRMKDDMEMLFAAAIDDEANNSMPTKEEQCLEAMNVMMRDGNLEAALDVKVEDLVGSPLDWDSQDLHDMVDQMGFLGSEP

SEQ ID NO: 76 - protein, 211 aa, Physcomitrella patens
MALSQSSTCSQVSGLVVHADLARPQSPKTQAPMSAVPVKADTAFQGTALRSVGRQTRSMAAPNVALKDLVASRDAEVGSSVSKLVSEGSEDLDSIATTSSDLSEVVDVVEEDAGGANIRVRKASGKAGTRTSRRRALVMCLALGMVRPISGNATGGLQAGNLRRTTSTNLRRSASSSFTVSGNLQSQVSIASSLKAANLLDDKLKNNVPTL

SEQ ID NO: 77 - protein, 250 aa, Physcomitrella patens
MADEYGRERIRDAVEGLGEDGPVVGGEVTDRGLFGRHGRHHGYNSGYSEEDAFASELGGPYGRRPPPGAVVYEGEGGFGDGYGRRPPVMPYEGVGGGYGGGYGNEYPPDVAGGGYGRHGYAGEDYGRRPGPPMYVEAPVENSDLGTGLVDSNIRTEPDYGAGYGRPDGTSAYEVQGRHGGKHGHLSKEEREELEDERKHKHYAEAAAAAALGYGLYERHEKRDAEDRLEELGYDSDGKKKQGHHFFRSDS

SEQ ID NO: 78 - protein, 210 aa, Physcomitrella patens
MALNSLASTSVIRGIALPAPFCDSTQLRRQAASPFVSRPRSYRTVVRSSRLPLNPKEAREMAEGREPERQNERGGNGGPNPFRFFQNFKDGLFQDHKRLQKEKSLPKGDLLYTVEKGDTLYAISERHECSLELLMEANGIEDPHNLSVGQEIWIPRTYQIKKGDTLYSISKHYGVSIEAIQAANGIDDPNFIHEGDHICLPEKTAHEDSD

SEQ ID NO: 79 - protein, 362 aa, Arabidopsis thaliana
MDNFLPFPSSNANSVQELSMDPNNNRSHFTTVPTYDHHQAQPHHFLPPFSYPVEQMAAVMNPQPVYLSECYPQIPVTQTGSEFGSLVGNPCLWQERGGFLDPRMTKMARINRKNAMMRSRNNSSPNSSPSELVDSKRQLMMLNLKNNVQISDKKDSYQQSTFDNKKLRVLCEKELKNSDVGSLGRIVLPKRDAEANLPKLSDKEGIVVQMRDVFSMQSWSFKYKFWSNNKSRMYVLENTGEFVKQNGAEIGDFLTIYEDESKNLYFAMNGNSGKQNEGRENESRERNHYEEAMLDYIPRDEEEASIAMLIGNLNDHYPIPNDLMDLTTDLQHHQATSSMTPEDHAYVGSSDDQVSFNDFEWW

SEQ ID NO: 80 - protein, 382 aa, Corynebacterium glutamicum
MTATYTTETAINFLFLSEPDMIAAGVKDVAQCVDVMEETLVLLAQGDYKMAGLNSNSHGAMITFPENPEFEGMPKDGPDRRFMAMPAYLGGRFKNTGVKWYGSNAENKASGLPRSIHTFVLNDTVTGAPKAIMSANLLSAYRTGAVPGVGVKHLAVADATTLAVVGPGVMAKTITEACIAERPGITTIKIKGRSERGINAFATWALEKFPEIEVVAVGSEEDVVKDADIVIAATTTDAAGSSAFPYFKKEWLKPGALLLLPAAGRFDDAYLLDDARLVVDYMGLYEAWAEEYGPQAYQLLGIPGTHWYDLALQGKLDLAKISQIGDICSGKLPGRTNDEEIILYSVGGMPVEDVAWATQVYENALEKGVGTTLNLWESPALA

SEQ ID NO: 81 - protein, 710 aa, Arabidopsis thaliana
MAATLPLCAALRSPVSSRRFAPIHKTDVPFQFNVVLSPFFGSVAIGGRIFPRLPAAKQETDQDEVGFDQQPSQELAIASACLVGVLTGVSVVLFNNCVHLLRDFSWDGIPDRGASWLREAPIGSNWLRVILVPTIGGLVVSILNQLRESAGKSTGDSHSSLDRVKAVLRPFLKTVAACVTLGTGNSLGPEGPSVEIGASIAKGVNSLFNKSPQTGFSLLAAGSAAGISSGFNAAVAGCFFAVESVLWPSSSTDSSTSLPNTTSMVILSAVTASVVSEIGLGSEPAFKVPDYDFRSPGELPLYLLLGALCGLVSLALSRCTSSMTSAVDSLNKDAGIPKAVFPVMGGLSVGIIALVYPEVLYWGFQNVDILLEKRPFVKGLSADLLLQLVAVKIAATAWCRASGLVGGYYAPSLFIGGAAGMAYGKFIGLALAQNPDFNLSILEVASPQAYGLVGMAATLAGVCQVPLTAVLLLFELTQDYRIVLPLLGAVGMSSWITSGQSKRQETRETKETRKRKSQEAVQSLTSSDDESSTNNLCEVESSLCLDDSLNQSEELPKSIFVSEAMRTRFATVMMSTSLEEALTRMLIEKQSCALIVDPDNIFLGILTLSDIQEFSKARKEGNNRPKDIFVNDICSRSGGKCKVPWTVTPDMDLLAAQTIMNKHELSHVAVVSGSIDAPRIHPVGVLDRECITLTRRALATRMYLLNSLYL

SEQ ID NO: 82 - protein, 249 aa, Arabidopsis thaliana
MASASSSDGVAGRIQNASLVLVSDNSSTLADIRKAVAMMKNIAVQLEKENQTDKVKDLENSVAELLDLHSDCNHRSTAIQSVANRYQPVEQLTDFKKLLDDEFTKLKATPSSVPQNDHLMRQFREAVWNVHHAGEPMPGDDDEDIVMTSTQCPLLNMTCPLSGKPVTELADPVRSMDCRHVYEKSVILHYIVNNPNANCPVAGCRGKLQNSKVICDAMLKFEIEEMRSLNKQSNRAEVIEDFTEDVDED

SEQ ID NO: 83 - protein, 281 aa, Arabidopsis thaliana
MSTSAASLCCSSTQVNGFGLRPERSLLYQPTSFSFSRRRTHGIVKASSRVDRFSKSDIIVSPSILSANFAKLGEQVKAVELAGCDWIHVDVMDGRFVPNITIGPLVVDALRPVTDLPLDVHLMIVEPEQRVPDFIKAGADIVSVHCEQQSTIHLHRTVNQIKSLGAKAGVVLNPGTPLSAIEYVLDMVDLVLIMSVNPGFGGQSFIESQVKKISDLRKMCAEKGVNPWIEVDGGVTPANAYKVIEAGANALVAGSAVFGAKDYAEAIKGIKASKRPAAVAV

SEQ ID NO: 84 - protein, 493 aa, Arabidopsis thaliana
MVLSKTVSESDVSIHSTFASRYVRNSLPRFEMPENSIPKEAAYQIINDELMLDGNPRLNLASFVTTWMEPECDKLMMESINKNYVDMDEYPVTTELQNRCVNMIARLFNAPLGDGEAAVGVGTVGSSEAIMLAGLAFKRQWQNKRKAQGLPYDKPNIVTGANVQVCWEKFARYFEVELKEVNLREDYYVMDPVKAVEMVDENTICVAAILGSTLTGEFEDVKLLNDLLVEKNKQTGWDTPIHVDAASGGFIAPFLYPELEWDFRLPLVKSINVSGHKYGLVYAGIGWVVWRTKTDLPDELIFHINYLGADQPTFTLNFSKGSSQVIAQYYQLIRLGFEGYRNVMDNCRENMMVLRQGLEKTGRFKIVSKENGVPLVAFSLKDSSRHNEFEVAHTLRRFGWIVPAYTMPADAQHVTVLRVVIREDFSRTLAERLVADFEKVLHELDTLPARVHAKMANGKVNGVKKTPEETQREVTAYWKKLLETKKTNKNTIC

SEQ ID NO: 85 - protein, 116 aa, Zea mays
MTETREINVFMAKLAEQAERYDEMVEAMKNVADLGQELTVEERNLLSVAYKNVIGARRASWRIITSIEQKEESKGNTAHVERIKEYRKKVENEVSKICADVLGTLDNKLIPNAQTT

SEQ ID NO: 86 - protein, 517 aa, Arabidopsis thaliana
MSPEAYVLFFNSFNLVTFEAFASVSLIIATVAFLLSPGGLAWAWTGSSKSRVSIPGPSGSLSVFSGSNPHRVLAALAKRFKASPLMAFSVGFSRFVISSEPETAKEILSSSAFADRPVKESAYELLFHRAMGFAPYGEYWRNLRRISSTHLFSPRRIASFEGVRVGIGMKMVKKIKSLVTSDACGEVEVKKIVHFGSLNNVMTTVFGESYDFDEVNGKGCFLERLVSEGYELLGIFNWSDHFWFLRWFDFQGVRKRCRALVSEVNTFVGGIIEKHKMKKGNNLNGEENDFVDVLLGLQKDEKLSDSDMIAVLWEMIFRGTDTVAILVEWVLARMVLHQDIQDKLYREIASATSNNIRSLSDSDIPKLPYLQAIVKETLRLHPPGPLLSWARLAIHDVHVGPNLVPAGTIAMVNMWSITHNAKIWTDPEAFMPERFISEDVSIMGSDLRLAPFGSGRRVCPGKAMGLATVHLWIGQLIQNFEWVKGSCDVELAEVLKLSMEMKNPLKCKAVPRNVGFA

SEQ ID NO: 87 - protein, 128 aa, Zea mays
MAASMISSSALAVAPQGLPPLGRRASSFAVVCSKKKIKTDKPYGIGGGLTVDVDANGRKGKGKGVYQFVDKYGANVDGYSPIYNEDDWSPTGDVYVGGTTGLLIWAVTLAGILGGGALLVYNTSALSG

SEQ ID NO: 88 - protein, 78 aa, Zea mays
MGGLSTKLFVVLLLLVCYTGTQGGPVTMVSARKCESQSFRFKGPCSRDANCANVCLTEGFTGGVCKGLRHRCFCTRDC

SEQ ID NO: 89 - DNA, 661 bp, Artificial sequence
  synthetic sequence for suppression of AMP1
aggagttgcgccagcaaaaaacccaggcccttgcaccgcacaatctacgttaagatatgccacagctttagcaccaagatttacaaggttctgctcaacccactccgtggaaccgatcattccaaattcttcagcatcccaactgcaaaaaatgattgtcctccgagggttccaaccctttcgcatcaaaagggcatatcttcgagcaatatcaaggagtgttgcagtcccactattggggtcaacagcaccataagtactgcgatcgcgttaacgctttatcacgataccttctaccacatatcactaacaacatcaacactcatcactctcgacgacatccactcgatcactactctcacacgaccgattaactcctcatccacgcggccgcctgcaggagcatggtgctgttgaccccaatagtgggactgcaacactccttgatattgctcgaagatatgcccttttgatgcgaaagggttggaaccctcggaggacaatcattttttgcagttgggatgctgaagaatttggaatgatcggttccacggagtgggttgagcagaaccttgtaaatcttggtgctaaagctgtggcatatcttaacgtagattgtgcggtgcaagggcctgggttttttgctggcgcaactccttag

SEQ ID NO: 90 - DNA, 11722 bp, Artificial sequence
  Artificial plasmid for corn transformation
cgcgcctgcctgcaggtactcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttagaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaagaaagaaaaaaaagaaaaagaaaaaacagcaggtgggtccgggtcgtgggggccggaaacgcgaggaggatcgcgagccagcgacgaggagcttaggcctcatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgaggcctcatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgaagctccctccctccgcttccaaagaaacgccccccatcgccactatatacatacccccccctctcctcccatccccccaacccttctagaaccatcttccacacactcaagccacactattggagaacacacagggacaacacaccataagatccaagggaggcctccgccgccgccggtaaccaccccgcccctctcctctttctttctccgtttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccgtgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaagcggaccggtcgcgcctcagcagtcgctgtcgttaacccagcggtactcgctgaggcgatcgcgggcccggtaccctgcaatgtgaccctagacttgtccatcttctggattggccaacttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattattattattattttgatgttcaatgaagttttttttaatttcatatgaagtatacaaaaattcttcatagatttttgtttctatgccgtagttatctttaatatatttgtggttgaagaaatttattgctagaaacgaatggattgtcaatttttttttaaagcaaatatatatgaaattatactgtatattattttagtcatgattaaaatgtggccttaattgaatcatctttctcattcattttttcaaaagcatatcaggatgattgatatttatctattttaaaaattaatttaagggttcaaattaaatttaacttaaaagtgtcctaaccgtagttaaaggtttactttaaaaaaatactatgaaaaatctaatcttctatgaatcgacctgcaggatttaaatccatcgttctggggcctaacgggccaagcttactcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttagaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcctcccccctccccctccgccgccgccgcgccggtaaccaccccgcccctctcctctttctttctccgtttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccgtgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaagtgatcaaccatggcgcaagttagcagaatctgcaatggtgtgcagaacccatctcttatctccaatctctcgaaatccagtcaacgcaaatctcccttatcggtttctctgaagacgcagcagcatccacgagcttatccgatttcgtcgtcgtggggattgaagaagagtgggatgacgttaattggctctgagcttcgtcctcttaaggtcatgtcttctgtttccacggcgtgcatgcttcacggtgcaagcagccggcccgcaaccgcccgcaaatcctctggcctttccggaaccgtccgcattcccggcgacaagtcgatctcccaccggtccttcatgttcggcggtctcgcgagcggtgaaacgcgcatcaccggccttctggaaggcgaggacgtcatcaatacgggcaaggccatgcaggcgatgggcgcccgcatccgtaaggaaggcgacacctggatcatcgatggcgtcggcaatggcggcctcctggcgcctgaggcgccgctcgatttcggcaatgccgccacgggctgccgcctgacgatgggcctcgtcggggtctacgatttcgacagcaccttcatcggcgacgcctcgctcacaaagcgcccgatgggccgcgtgttgaacccgctgcgcgaaatgggcgtgcaggtgaaatcggaagacggtgaccgtcttcccgttaccttgcgcgggccgaagacgccgacgccgatcacctaccgcgtgccgatggcctccgcacaggtgaagtccgccgtgctgctcgccggcctcaacacgcccggcatcacgacggtcatcgagccgatcatgacgcgcgatcatacggaaaagatgctgcagggctttggcgccaaccttaccgtcgagacggatgcggacggcgtgcgcaccatccgcctggaaggccgcggcaagctcaccggccaagtcatcgacgtgccgggcgacccgtcctcgacggccttcccgctggttgcggccctgcttgttccgggctccgacgtcaccatcctcaacgtgctgatgaaccccacccgcaccggcctcatcctgacgctgcaggaaatgggcgccgacatcgaagtcatcaacccgcgccttgccggcggcgaagacgtggcggacctgcgcgttcgctcctccacgctgaagggcgtcacggtgccggaagaccgcgcgccttcgatgatcgacgaatatccgattctcgctgtcgccgccgccttcgcggaaggggcgaccgtgatgaacggtctggaagaactccgcgtcaaggaaagcgaccgcctctcggccgtcgccaatggcctcaagctcaatggcgtggattgcgatgagggcgagacgtcgctcgtcgtgcgtggccgccctgacggcaaggggctcggcaacgcctcgggcgccgccgtcgccacccatctcgatcaccgcatcgccatgagcttcctcgtcatgggcctcgtgtcggaaaaccctgtcacggtggacgatgccacgatgatcgccacgagcttcccggagttcatggacctgatggccgggctgggcgcgaagatcgaactctccgatacgaaggctgcctgatgagctcgaattcccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggggatgggggatccactagtgatatccgtcgactggtacctacgcgtagctagcccgtgaagtttctcatctaagcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctgatccatgtagatttcccggacatgaagccatttacaattgaatatatcctgccgccgctgccgctttgcacccggtggagcttgcatgttggtttctacgcagaactgagccggttaggcagataatttccattgagaactgagccatgtgcaccttccccccaacacggtgagcgacggggcaacggagtgatccacatgggacttttcctagcttggctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccggccgcggtgtctcgcacacggcttcgacggcgtttctggcgcgtttgcagggccatagacggccgccagcccagcggcgagggcaaccagcccggtgagcgtcggaaagggtcgatcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggtcgggagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataatgtctaacaattcgttcaagccgacgccgcttcgcggcgcggcttaactcaagcgttagatgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgcacggaatgccaagcactcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgg

SEQ ID NO: 91 - DNA, 9769 bp, Artificial sequence
  Artificial plasmid for soybean transformation
ggtccgatgtgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatggtccgattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggaccaggtggtaccggcgcgcctcagcagtcgctgtcgttaacccagcggtactcgctgaggcgatcgcgggccctgatcacctgtcgtacagtatttctacatttgatgtgtgatttgtgaagaacatcaaacaaaacaagcactggctttaatatgatgataagtattatggtaattaattaattggcaaaaacaacaatgaagctaaaattttatttattgagccttgcggttaatttcttgtgatgatctttttttttattttctaattatatatagtttcctttgctttgaaatgctaaaggtttgagagagttatgctctttttttcttcctctttcttttttaactttatcatacaaattttgaataaaaatgtgagtacattgagctcatttaaataagcttgatggggatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggagtgcttggcattccgtgcgataatgacttctgttcaaccacccaaacgtcggaaagcctgacgacggagcagcattccaaaaagatcccttggctcgtctgggtcggctagaaggtcgagtgggctgctgtggcttgatccctcaacgcggtcgcggacgtagcgcagcgccgaaaaatcctcgatcggaggaccgaaggagctaaccgcttttttgcacaacatgggggatcatgtaactcgccttgatcgttgggaaccggagctgaatgaagccataccaaacgacgagcgtgacaccacgatgcctgcagcatctaacgcttgagttaagccgcgccgcgaagcggcgtcggcttgaacgaattgttagacattatttgccgactaccttggtgatctcgcctttcacgtagtgaacaaattcttccaactgatctgcgcgcgaggccaagcgatcttcttgtccaagataagcctgcctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactcctgaattaagccgcgccgcgaagcggtgtcggcttgaatgaattgttaggcgtcatcctgtgctcccgacctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgatcgaccctttccgacgctcaccgggctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagccaagctaggaaaagtcccatgtggatcactccgttgccccgtcgctcaccgtgttggggggaaggtgcacatggctcagttctcaatggaaattatctgcctaaccggctcagttctgcgtagaaaccaacatgcaagctccaccgggtgcaaagcggcagcggcggcaggatatattcaattgtaaatggcttcatgtccgggaaatctacatggatcagcaatgagtatgatggtcaatatggagaaaaagaaagagtaattaccaattttttttcaattcaaaaatgtagatgtccgcagcgttattataaaatgaaagtacattttgataaaacgacaaattacgatccgtcgtatttataggcgaaagcaataaacaaattattctaattcggaaatctttatttcgacgtgtctacattcacgtccaaatgggggcttagatgagaaacttcacgatcgatgcggccaccactcgagaagcttactagtcaacaattggccaatctttgttctaaattgctaataaacgaccatttccgtcaattctccttggttgcaacagtctacccgtcaaatgtttactaatttataagtgtgaagtttgaattatgaaagacgaaatcgtattaaaaattcacaagaataaacaactccatagattttcaaaaaaacagtcacgagaaaaaaaccacagtccgtttgtctgctcttctagtttttattatttttctattaatagttttttgttatttcgagaataaaatttgaacgatgtccgaaccacaaaagccgagccgataaatcctaagccgagcctaactttagccgtaaccatcagtcacggctcccgggctaattcatttgaaccgaatcataatcaacggtttagatcaaactcaaaacaatctaacggcaacatagacgcgtcggtgagctaaaaagagtgtgaaagccaggtcaccatagcattgtctctcccagattttttatttgggaaataatagaagaaatagaaaaaaataaaagagtgagaaaaatcgtagagctatatattcgcacatgtactcgtttcgctttccttagtgttagctgctgccgctgttgtttctcctccatttctctatctttctctctcgctgcttctcgaatcttctgtatcatcttcttcttcttcaaggtgagtctctagatccgttcgcttgattttgctgctcgttagtcgttattgttgattctctatgccgatttcgctagatctgtttagcatgcgttgtggttttatgagaaaatctttgttttgggggttgcttgttatgtgattcgatccgtgcttgttggatcgatctgagttaattcttaaggtttatgtgttagatctatggagtttgaggattcttctcgcttctgtcgatctctcgctgttatttttgtttttttcagtgaagtgaagttgtttagttcgaaatgacttcgtgtatgctcgattgatctggttttaatcttcgatctgttaggtgttgatgtttacaagtgaattctagtgttttctcgttgagatctgtgaagtttgaacctagttttctcaataatcaacatatgaagcgatgtttgagtttcaataaacgctgctaatcttcgaaactaagttgtgatctgattcgtgtttacttcatgagcttatccaattcatttcggtttcattttactttttttttagtgaaccatggcgcaagttagcagaatctgcaatggtgtgcagaacccatctcttatctccaatctctcgaaatccagtcaacgcaaatctcccttatcggtttctctgaagacgcagcagcatccacgagcttatccgatttcgtcgtcgtggggattgaagaagagtgggatgacgttaattggctctgagcttcgtcctcttaaggtcatgtcttctgtttccacggcgtgcatgcttcatggagcttcatctaggccagctactgccaggaagtctagcgggctcagtggcaccgtgcgcatccctggcgataaaagtatttcacacaggagcttcatgttcggaggacttgctagtggagagacgagaatcactggtttgcttgagggcgaagatgttatcaacaccggtaaggcgatgcaagcaatgggtgccagaatccgaaaagagggcgatacgtggatcatcgacggtgttggtaacggaggattgctcgctcccgaagcgccacttgactttgggaacgcagctacggggtgccgtcttactatgggactggtaggcgtgtatgactttgactctaccttcatcggtgacgcgagcctcactaagagaccaatgggacgagtgctgaatcccctgagggagatgggtgtccaggtgaaatctgaggatggtgatcgtcttccggttactctgcgaggccccaagacccccacgccaatcacgtacagggttccgatggcgtcagcacaggtcaagtcagcggtactcctggcgggcctcaacacacctggaatcacaaccgtgattgaacccatcatgactagagaccacacggagaagatgttgcagggtttcggcgctaatctaacggtcgaaaccgacgccgacggcgtgaggacaatccgcttggagggcagaggtaaactgactggccaagtcatcgatgtgcctggagatccctcgtccacagcgtttcccctcgtagctgcgttgctcgtccctggatctgatgtgacgatcctgaatgtcctcatgaatccaactagaaccggcctcatcctcacattgcaggagatgggtgctgacatcgaggttatcaatcctaggttggcaggtggagaggatgtggccgatctgcgcgtgcgttctagtacactcaaaggcgtgaccgtccctgaggatcgcgctccatccatgatcgacgagtaccccattctcgccgttgctgctgcgtttgccgagggcgcaactgtaatgaacggccttgaggagttgagggttaaggagagtgacaggctgtccgcggtggcgaatggcctgaagctaaacggcgtggactgcgacgaaggtgaaacgtcccttgtagtccgtggtcgcccagacgggaaggggttggggaatgcttcgggagctgctgtggcgacgcaccttgatcatagaatcgccatgtcatttctggtgatgggacttgtctccgagaatccggtgaccgttgacgatgctaccatgatcgccacctcctttcctgagttcatggacctcatggcaggcttgggggccaagatcgagctgtctgatactaaggccgcttgaattcccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggggatcccacgtgcggaccgcctgcaggccgcgttatcaagctaactgca

SEQ ID NO: 92 - DNA, 8504 bp, Artificial sequence
  Artificial plasmid for cotton transformation
aggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgcacggaatgccaagcactcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatccccatcaagcttggccagcttctgcaggtccgattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatggtccgattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacagaaaaatttgctacattgtttcacaaacttcaaatattattcatttatttgtcagctttcaaactctttgtttcttgtttgttgattagatctggtaccctcagcagtcgctgtgcgatcgccagcggtactcgctgaggtcgacgtagttagttaattcagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacgcggccgccactcgagtggaagctagctttccgatcctacctgtcacttcatcaaaaggacagtagaaaaggaaggtggcacctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgatacttccactgacgtaagggatgacgcacaatcccactatccttcgcaagacccttcctctatataaggaagttcatttcatttggagaggacacgctgaaatcaccagtctctctctacaagatcggggatctctagctagacgatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgatccccaattcccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggggatcgggccactcgagtggtggccgcatcgatcgtgaagtttctcatctaagcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctgatccatgtagatttcccggacatgaagccatttacaattgaatatatcctgccgccgctgccgctttgcacccggtggagcttgcatgttggtttctacgcagaactgagccggttaggcagataatttccattgagaactgagccatgtgcaccttccccccaacacggtgagcgacggggcaacggagtgatccacatgggacttttcctagcttggctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccggccgcggtgtctcgcacacggcttcgacggcgtttctggcgcgtttgcagggccatagacggccgccagcccagcggcgagggcaaccagcccggtgagcgtcggaaagggtcgatcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggtcgggagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataatgtctaacaattcgttcaagccgacgccgcttcgcggcgcggcttaactcaagcgttagatgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcg

(19) United States
(12) Patent Application Publication
(10) Pub. No.: US 2013/0145493 A1
(43) Pub. Date: Jun. 6, 2013

(54) Transgenic Plants with Enhanced Agronomic Traits

(71) Applicants: Edwards Allen, O'Fallon, MO (US); Veena S. Anil, Bangalore (IN); Amarjit Basra, Chesterfield, MO (US); Christopher T. Bauer, Edwardsville, IL (US); Prasanna R. Bhat, Bangalore (IN); Raghunatha P. Chari, Andhar Pradesh (IN); Jaishree Chittoor, Wildwood, MO (US); Paul Chomet, Mystic, CT (US); Karen K. Gabbert, St. Louis, MO (US); Cara Griffith, Catawissa, MO (US); Bill Hendrix, West Sacramento, CA (US); Alberto Iandolino, Davis, CA (US); Hongwu Jia, Grover, MO (US); Kevin R. Kosola, Wildwood, MO (US); Saritha V. Kuriakose, Kottayam (IN); Paul J. Loida, Kirkwood, MO (US); Linda L. Lutfiyya, St. Louis, MO (US); Mingsheng Peng, Wildwood, MO (US); Ying Peng, Davis, CA (US); Bellur Narasimha Prasad, Virupakshapura (IN); Monnanda S. Rajani, Chesterfield, MO (US); Monica P. Ravanello, Fairfield, CA (US); Donald Todd, St. Louis, MO (US); Srikanth Babu Venkatachalayya, Bangalore (IN); Tyanamagondlu V. Venkatesh, St. Louis, MO (US); K. R. Vidya, Malleswaram (IN); Huai Wang, Chesterfield, MO (US)

(72) Inventors: Edwards Allen, O'Fallon, MO (US); Veena S. Anil, Bangalore (IN); Amarjit Basra, Chesterfield, MO (US); Christopher T. Bauer, Edwardsville, IL (US); Prasanna R. Bhat, Bangalore (IN); Raghunatha P. Chari, Andhar Pradesh (IN); Jaishree Chittoor, Wildwood, MO (US); Paul Chomet, Mystic, CT (US); Karen K. Gabbert, St. Louis, MO (US); Cara Griffith, Catawissa, MO (US); Bill Hendrix, West Sacramento, CA (US); Alberto Iandolino, Davis, CA (US); Hongwu Jia, Grover, MO (US); Kevin R. Kosola, Wildwood, MO (US); Saritha V. Kuriakose, Kottayam (IN); Paul J. Loida, Kirkwood, MO (US); Linda L. Lutfiyya, St. Louis, MO (US); Mingsheng Peng, Wildwood, MO (US); Ying Peng, Davis, CA (US); Bellur Narasimha Prasad, Virupakshapura (IN); Monnanda S. Rajani, Chesterfield, MO (US); Monica P. Ravanello, Fairfield, CA (US); Donald Todd, St. Louis, MO (US); Srikanth Babu Venkatachalayya, Bangalore (IN); Tyanamagondlu V. Venkatesh, St. Louis, MO (US); K. R. Vidya, Malleswaram (IN); Huai Wang, Chesterfield, MO (US)

(21) Appl. No.: 13/520,822

(22) PCT Filed: Jan. 12, 2011
(86) PCT No.: PCT/US11/20918

§ 371 Date: Nov. 6, 2012

Related U.S. Application Data
(60) Provisional application No. 61/294,369, filed on Jan. 12, 2010.
(60) Provisional application No. 61/313,170, filed on Mar. 12, 2010.
(60) Provisional application No. 61/346,724, filed on May 20, 2010.

(51) Int. Cl. C12N 15/82

(52) U.S. Cl. CPC C12N 15/8273; C12N 15/8271
USPC 800/275; 800/298; 800/320.1; 800/312; 800/314; 800/306; 800/320.3; 800/320.2; 435/320.1; 435/419

(57) ABSTRACT

This invention provides transgenic plant cells with recombinant DNA for expression of proteins that are useful for imparting enhanced agronomic trait(s) to transgenic crop plants. This invention also provides transgenic plants and progeny seed comprising the transgenic plant cells where the plants are selected for having an enhanced trait selected from the group of traits consisting of enhanced water use efficiency, enhanced cold tolerance, increased yield, enhanced nitrogen use efficiency, enhanced seed protein and enhanced seed oil. Also disclosed are methods for manufacturing transgenic seed and plants with enhanced traits

### CROSS REFERENCE TO RELATED APPLICATIONS

This application claims benefit to U.S. provisional application Ser. No. 61/294,369, filed Jan. 12, 2010, U.S. provisional application Ser. No. 61/313,170, filed Mar. 12, 2010 and U.S. provisional application Ser. No. 61/346,724, filed May 20, 2010, all of which are herein incorporated by reference.

### INCORPORATION OF SEQUENCE LISTING

The sequence listing file named “38_{—}21_{—}57089_A_PCT_seq_listing.txt”, which is 240,706 bytes (measured in MS-WINDOWS) which is filed electronically herewith and which was created on Jan. 11, 2011 is incorporated herein by reference.

### FIELD OF THE INVENTION

Disclosed herein are recombinant DNA useful for providing enhanced traits to transgenic plants, seeds, pollen, plant cells and plant nuclei of such transgenic plants, methods of making and using such recombinant DNA, plants, seeds, pollen, plant cells and plant nuclei. Also disclosed are methods of producing hybrid corn seed comprising such recombinant DNA.

All genetic resources disclosed herein were directly obtained from sources that are currently common to the United States; the ancestral sources of each specific genetic material is unknown.

### SUMMARY OF THE INVENTION

Yet another aspect of this invention provides recombinant DNA constructs comprising polynucleotides characterized by reference to SEQ ID NO:1-44 and the cognate proteins with amino acid sequences having reference to SEQ ID NO:45-88. The recombinant DNA constructs are useful for providing enhanced traits when stably integrated into the chromosomes and expressed in the nuclei of transgenic plants cells. In some aspects of the invention the recombinant DNA constructs, when expressed in a plant cell, provide for expression of cognate proteins. In those aspects of the invention, the recombinant DNA constructs for expressing cognate proteins are characterized by cognate amino acid sequences having a sequence selected from SEQ ID NOs: 45-68, and 70-88; having at least 90% identity over at least 90% of the length of a sequence selected from the group consisting of SEQ ID NOs: 45-68, and 70-88 or that are homologous to a sequence selected from the group consisting of SEQ ID NOs: 45-68, and 70-88.

In other aspects of the invention the recombinant DNA constructs provide for suppression of a native protein. In those other aspects of the invention the recombinant DNA constructs are characterized as being constructed with sense-oriented and anti-sense-oriented polynucleotides, e.g. polynucleotides derived from genes having SEQ ID NO: 25 or homologous genes. When the recombinant DNA construct is expressed in corn plants, the endogenous protein is the corn homolog of SEQ ID NO:69; when the recombinant DNA construct is expressed in soybean plants, the endogenous protein is a soybean homolog of SEQ ID NO: 69; and when the recombinant DNA construct is expressed in a plant other than a corn or a soybean plant, the endogenous protein is the other plant's endogenous protein that has an amino acid sequence homologous to SEQ ID NO: 69.

In practical aspects of this invention the recombinant DNA constructs of the invention are stably integrated into the chromosome of a plant cell nucleus.

This invention also provides transgenic plant cells comprising the stably integrated recombinant DNA constructs of the invention, transgenic plants and seeds comprising a plurality of such transgenic plant cells and transgenic pollen of such plants. Such transgenic plants are selected from a population of transgenic plants regenerated from plant cells transformed with recombinant DNA constructs by screening transgenic plants for an enhanced trait as compared to control plants. The enhanced trait is one or more of enhanced water use efficiency, enhanced cold tolerance, increased yield, enhanced nitrogen use efficiency, enhanced seed protein and enhanced seed oil.

In another aspect of the invention the plant cells, plants, seeds, and pollen further comprise DNA expressing a protein that provides tolerance from exposure to an herbicide applied at levels that are lethal to a wild type plant cell.

This invention also provides methods for manufacturing non-natural, transgenic seed that can be used to produce a crop of transgenic plants with an enhanced trait resulting from expression of a stably-integrated recombinant DNA construct. More specifically, the method comprises (a) screening a population of plants for an enhanced trait and a recombinant DNA construct, where individual plants in the population can exhibit the trait at a level less than, essentially the same as or greater than the level that the trait is exhibited in control plants, (b) selecting from the population one or more plants that exhibit the trait at a level greater than the level that said trait is exhibited in control plants, (c) collecting seed from a selected plant, (d) verifying that the recombinant DNA is stably integrated in said selected plants, (e) analyzing tissue of a selected plant to determine the production or suppression of a protein having the function of a protein encoded by nucleotides in a sequence of one of SEQ ID NOs: 1-44. In one aspect of the invention, the plants in the population further comprise DNA expressing a protein that provides tolerance to exposure to a herbicide applied at levels that are lethal to wild type plant cells and the selecting is affected by treating the population with the herbicide, e.g. a glyphosate, dicamba, or glufosinate compound. In another aspect of the invention the plants are selected by identifying plants with the enhanced trait. The methods are especially useful for manufacturing corn, soybean, cotton, canola, alfalfa, wheat, rice, sugarcane or sugar beet seed.

Another aspect of the invention provides a method of producing hybrid corn seed comprising acquiring hybrid corn seed from a herbicide tolerant corn plant which also has stably-integrated, recombinant DNA construct comprising a promoter that is (a) functional in plant cells and (b) is operably linked to DNA that encodes or suppresses a protein having the function of a protein encoded by nucleotides in a sequence of one of SEQ ID NOs: 1-44. The methods further comprise producing corn plants from said hybrid corn seed, wherein a fraction of the plants produced from said hybrid corn seed is homozygous for said recombinant DNA, a fraction of the plants produced from said hybrid corn seed is hemizygous for said recombinant DNA, and a fraction of the plants produced from said hybrid corn seed has none of said recombinant DNA; selecting corn plants which are homozygous and hemizygous for said recombinant DNA by treating with an herbicide; collecting seed from herbicide-treated-surviving corn plants and planting said seed to produce further progeny corn plants; repeating the selecting and collecting steps at least once to produce an inbred corn line; and crossing the inbred corn line with a second corn line to produce hybrid seed.

Another aspect of the invention provides a method of selecting a plant comprising plant cells of the invention by using an immunoreactive antibody to detect the presence or absence of protein expressed or suppressed by recombinant DNA in seed or plant tissue. Yet another aspect of the invention provides anti-counterfeit milled seed having, as an indication of origin, plant cells of this invention.

Still other aspects of this invention relate to transgenic plants with enhanced water use efficiency or enhanced nitrogen use efficiency. For instance, this invention provides methods of growing a corn, cotton, soybean, or canola crop without irrigation water comprising planting seed having plant cells of the invention which are selected for enhanced water use efficiency. Alternatively methods comprise applying reduced irrigation water, e.g. providing up to 300 millimeters of ground water during the production of a corn crop. This invention also provides methods of growing a corn, cotton, soybean or canola crop without added nitrogen fertilizer comprising planting seed having plant cells of the invention which are selected for enhanced nitrogen use efficiency.

### DETAILED DESCRIPTION OF THE INVENTION

In the attached sequence listing:

SEQ ID NO:1-44 are nucleotide sequences of the coding strand of DNA for “genes” used in the recombinant DNA imparting an enhanced trait in plant cells, i.e. each represents a coding sequence for a protein;

SEQ ID NO: 45-88 are amino acid sequences of the cognate protein of the “genes” with nucleotide coding sequences 1-44;

SEQ ID NO: 89 is a DNA sequence which, when expressed in plant cells, suppresses the expression of AMP1 (SEQ ID NO: 69).

SEQ ID NO: 90 is a nucleotide sequence of a base plasmid vector useful for corn transformation;

SEQ ID NO: 91 is a nucleotide sequence of a base plasmid vector useful for soybean and canola transformation;

SEQ ID NO: 92 is a nucleotide sequence of a base plasmid vector useful for cotton transformation;

As used herein a “plant cell” means a plant cell that is transformed with stably-integrated, non-natural, recombinant DNA, e.g. by Agrobacterium-mediated transformation or by bombardment using microparticles coated with recombinant DNA or other means. A plant cell of this invention can be an originally-transformed plant cell that exists as a microorganism or as a progeny plant cell that is regenerated into differentiated tissue, e.g. into a transgenic plant with stably-integrated, non-natural recombinant DNA, or seed or pollen derived from a progeny transgenic plant.

As used herein a “transgenic plant” means a plant whose genome has been altered by the stable integration of recombinant DNA. A transgenic plant includes a plant regenerated from an originally-transformed plant cell and progeny transgenic plants from later generations or crosses of a transformed plant.

As used herein “recombinant DNA” means DNA which has been a genetically engineered and constructed outside of a cell including DNA containing naturally occurring DNA or cDNA or synthetic DNA.

As used herein a “homolog” means a protein in a group of proteins that perform the same biological function, i.e. proteins that provide a common enhanced trait in transgenic plants of this invention. Homologs are expressed by homologous genes. With reference to homologous genes, homologs include orthologs, i.e. genes expressed in different species that evolved from a common ancestral genes by speciation and encode proteins retain the same function, but do not include paralogs, i.e. genes that are related by duplication but have evolved to encode proteins with different functions. Homologous genes include naturally occurring alleles and artificially-created variants. Degeneracy of the genetic code provides the possibility to substitute at least one base of the protein encoding sequence of a gene with a different base without causing the amino acid sequence of the polypeptide produced from the gene to be changed. When optimally aligned, homolog proteins have at least 60% identity, 65% identity, 70% identity, 75% identity, 80%, identity, 85% identity, 90% identity, 95, 96, 97, 98, or 99% identity over the full length of a protein identified as being associated with imparting an enhanced trait when expressed in plant cells.

Homologs are identified by comparison of amino acid sequence, e.g. manually or by use of a computer-based tool using known homology-based search algorithms such as the suite of BLAST programs available from NCBI. A local sequence alignment program, e.g. BLAST, can be used to search a database of sequences to find similar sequences, and the summary Expectation value (E-value) used to measure the sequence base similarity. Because a protein hit with the best E-value for a particular organism may not necessarily be an ortholog, i.e. have the same function, or be the only ortholog, a reciprocal query is used to filter hit sequences with significant E-values for ortholog identification. The reciprocal query entails search of the significant hits against a database of amino acid sequences from the base organism that are similar to the sequence of the query protein. A hit can be identified as an ortholog, when the reciprocal query's best hit is the query protein itself or a protein encoded by a duplicated gene after speciation. A further aspect of the homologs encoded by DNA useful in the transgenic plants of the invention are those proteins that differ from a disclosed protein as the result of deletion or insertion of one or more amino acids in a native sequence.

Percent identity describes the extent to which the sequences of DNA or protein segments are invariant in an alignment of sequences, for example nucleotide sequences or amino acid sequences. An alignment of sequences is created by manually aligning two sequences, e.g. a stated sequence, as provided herein, as a reference, and another sequence, to produce the highest number of matching elements, e.g. individual nucleotides or amino acids, while allowing for the introduction of gaps into either sequence. An “identity fraction” for a sequence aligned with a reference sequence is the number of matching elements, divided by the full length of the reference sequence, not including gaps introduced by the alignment process into the reference sequence. “Percent identity” (“% identity”) as used herein is the identity fraction times 100.

As used herein “promoter” means regulatory DNA for initializing transcription. A “plant promoter” is a promoter capable of initiating transcription in plant cells whether or not its origin is a plant cell, e.g. is it well known that Agrobacterium promoters are functional in plant cells. Thus, plant promoters include promoter DNA obtained from plants, plant viruses and bacteria such as Agrobacterium and Bradyrhizobium bacteria. Examples of promoters under developmental control include promoters that preferentially initiate transcription in certain tissues, such as leaves, roots, or seeds. Such promoters are referred to as “tissue preferred”. Promoters that initiate transcription only in certain tissues are referred to as “tissue specific”. A “cell type” specific promoter primarily drives expression in certain cell types in one or more organs, for example, vascular cells in roots or leaves. An “inducible” or “repressible” promoter is a promoter which is under environmental control. Examples of environmental conditions that may effect transcription by inducible promoters include anaerobic conditions, or certain chemicals, or the presence of light. Tissue specific, tissue preferred, cell type specific, and inducible promoters constitute the class of “non-constitutive” promoters. A “constitutive” promoter is a promoter which is active under most conditions.

As used herein “operably linked” means the association of two or more DNA fragments in a recombinant DNA construct so that the function of one, e.g. protein-encoding DNA, is controlled by the other, e.g. a promoter.

As used herein “expressed” means produced, e.g. a protein is expressed in a plant cell when its cognate DNA is transcribed to mRNA that is translated to the protein.

As used herein “suppressed” means decreased, e.g. a protein is suppressed in a plant cell when there is a decrease in the amount and/or activity of the protein in the plant cell. The presence or activity of the protein can be decreased by any amount up to and including a total loss of protein expression and/or activity.

“Arabidopsis” means plants of Arabidopsis thaliana.

As used herein a “control plant” means a plant that does not contain the recombinant DNA that imparts an enhanced trait. A control plant is used to identify and select a transgenic plant that has an enhanced trait. A suitable control plant can be a non-transgenic plant of the parental line used to generate a transgenic plant, i.e. devoid of recombinant DNA. A suitable control plant may in some cases be a progeny of a hemizygous transgenic plant line that does not contain the recombinant DNA, known as a negative segregant.

As used herein an “enhanced trait” means a characteristic of a transgenic plant that includes, but is not limited to, an enhance agronomic trait characterized by enhanced plant morphology, physiology, growth and development, yield, nutritional enhancement, disease or pest resistance, or environmental or chemical tolerance. In more specific aspects of this invention enhanced trait is selected from group of enhanced traits consisting of enhanced water use efficiency, enhanced cold tolerance, increased yield, enhanced nitrogen use efficiency, enhanced seed protein and enhanced seed oil. In an important aspect of the invention the enhanced trait is enhanced yield including increased yield under non-stress conditions and increased yield under environmental stress conditions. Stress conditions may include, for example, drought, shade, fungal disease, viral disease, bacterial disease, insect infestation, nematode infestation, cold temperature exposure, heat exposure, osmotic stress, reduced nitrogen nutrient availability, reduced phosphorus nutrient availability and high plant density. “Yield” can be affected by many properties including without limitation, plant height, pod number, pod position on the plant, number of internodes, incidence of pod shatter, grain size, efficiency of nodulation and nitrogen fixation, efficiency of nutrient assimilation, resistance to biotic and abiotic stress, carbon assimilation, plant architecture, resistance to lodging, percent seed germination, seedling vigor, and juvenile traits. Yield can also be affected by efficiency of germination (including germination in stressed conditions), growth rate (including growth rate in stressed conditions), ear number, seed number per ear, seed size, composition of seed (starch, oil, protein) and characteristics of seed fill.

Increased yield of a transgenic plant of the present invention can be measured in a number of ways, including test weight, seed number per plant, seed weight, seed number per unit area (i.e. seeds, or weight of seeds, per acre), bushels per acre, tons per acre, or kilo per hectare. For example, corn yield may be measured as production of shelled corn kernels per unit of production area, for example in bushels per acre or metric tons per hectare, often reported on a moisture adjusted basis, for example at 15.5 percent moisture. Increased yield may result from improved utilization of key biochemical compounds, such as nitrogen, phosphorous and carbohydrate, or from improved responses to environmental stresses, such as cold, heat, drought, salt, and attack by pests or pathogens. Recombinant DNA used in this invention can also be used to provide plants having improved growth and development, and ultimately increased yield, as the result of modified expression of plant growth regulators or modification of cell cycle or photosynthesis pathways. Also of interest is the generation of transgenic plants that demonstrate enhanced yield with respect to a seed component that may or may not correspond to an increase in overall plant yield. Such properties include enhancements in seed oil, seed molecules such as protein and starch, oil components as may be manifest by an alterations in the ratios of seed components.

Recombinant DNA constructs are assembled using methods well known to persons of ordinary skill in the art and typically comprise a promoter operably linked to DNA, the expression of which provides the enhanced agronomic trait. Other construct components may include additional regulatory elements, such as 5′ leaders and introns for enhancing transcription, 3′ untranslated regions (such as polyadenylation signals and sites), DNA for transit or signal peptides.

Numerous promoters that are active in plant cells have been described in the literature. These include promoters present in plant genomes as well as promoters from other sources, including nopaline synthase (NOS) promoter and octopine synthase (OCS) promoters carried on tumor-inducing plasmids of Agrobacterium tumefaciens and the CaMV35S promoters from the cauliflower mosaic virus as disclosed in U.S. Pat. Nos. 5,164,316 and 5,322,938. Useful promoters derived from plant genes are found in U.S. Pat. No. 5,641,876 which discloses a rice actin promoter, U.S. Pat. No. 7,151,204 which discloses a maize chloroplast aldolase promoter and a maize aldolase (FDA) promoter, and US Patent Application Publication 2003/0131377 A1 which discloses a maize nicotianamine synthase promoter. These and numerous other promoters that function in plant cells are known to those skilled in the art and available for use in recombinant polynucleotides of the present invention to provide for expression of desired genes in transgenic plant cells.

Furthermore, the promoters may be altered to contain multiple “enhancer sequences” to assist in elevating gene expression. Such enhancers are known in the art. By including an enhancer sequence with such constructs, the expression of the selected protein may be enhanced. These enhancers often are found 5′ to the start of transcription in a promoter that functions in eukaryotic cells, but can often be inserted upstream (5′) or downstream (3′) to the coding sequence. In some instances, these 5′ enhancing elements are introns. Particularly useful as enhancers are the 5′ introns of the rice actin 1 (see U.S. Pat. No. 5,641,876) and rice actin 2 genes, the maize alcohol dehydrogenase gene intron, the maize heat shock protein 70 gene intron (U.S. Pat. No. 5,593,874) and the maize shrunken 1 gene. See also US Patent Application Publication 2002/0192813A1 which discloses 5′, 3′ and intron elements useful in the design of effective plant expression vectors.

In other aspects of the invention, sufficient expression in plant seed tissues is desired to affect improvements in seed composition. Exemplary promoters for use for seed composition modification include promoters from seed genes such as napin as disclosed in U.S. Pat. No. 5,420,034, maize L3 oleosin as disclosed in U.S. Pat. No. 6,433,252), zein Z27 as disclosed by Russell et al. (1997) Transgenic Res. 6(2):157-166), globulin 1 as disclosed by Belanger et al (1991) Genetics 129:863-872), glutelin 1 as disclosed by Russell (1997) supra), and peroxiredoxin antioxidant (Per1) as disclosed by Stacy et al. (1996) Plant Mol Biol. 31(6):1205-1216.

Recombinant DNA constructs useful in this invention will also generally include a 3′ element that typically contains a polyadenylation signal and site. Well-known 3′ elements include those from Agrobacterium tumefaciens genes such as nos 3, tml 3, tmr 3, tms 3, ocs 3, tr7 3, for example disclosed in U.S. Pat. No. 6,090,627; 3′ elements from plant genes such as wheat (Triticum aesevitum) heat shock protein 17 (Hsp17 3′), a wheat ubiquitin gene, a wheat fructose-1,6-biphosphatase gene, a rice glutelin gene, a rice lactate dehydrogenase gene and a rice beta-tubulin gene, all of which are disclosed in US Patent Application Publication 2002/0192813 A1; and the pea (Pisum sativum) ribulose biphosphate carboxylase gene (rbs 3′), and 3′ elements from the genes within the host plant.

Constructs and vectors may also include a transit peptide for targeting of a gene to a plant organelle, particularly to a chloroplast, leucoplast or other plastid organelle. For descriptions of the use of chloroplast transit peptides see U.S. Pat. No. 5,188,642 and U.S. Pat. No. 5,728,925. For description of the transit peptide region of an Arabidopsis EPSPS gene useful in the present invention, see Klee, H. J. et al (MGG (1987) 210:437-442).

Recombinant DNA constructs for gene suppression can be designed for any of a number the well-known methods for suppressing transcription of a gene, the accumulation of the mRNA corresponding to that gene or preventing translation of the transcript into protein. Posttranscriptional gene suppression can be practically effected by transcription of RNA that forms double-stranded RNA (dsRNA) having homology to mRNA produced from a gene targeted for suppression.

Gene suppression can also be achieved by insertion mutations created by transposable elements may also prevent gene function. For example, in many dicot plants, transformation with the T-DNA of Agrobacterium may be readily achieved and large numbers of transformants can be rapidly obtained. Also, some species have lines with active transposable elements that can efficiently be used for the generation of large numbers of insertion mutations, while some other species lack such options. Mutant plants produced by Agrobacterium or transposon mutagenesis and having altered expression of a polypeptide of interest can be identified using the polynucleotides of the present invention. For example, a large population of mutated plants may be screened with polynucleotides encoding the polypeptide of interest to detect mutated plants having an insertion in the gene encoding the polypeptide of interest.

Transgenic plants may comprise a stack of one or more polynucleotides disclosed herein resulting in the production or suppression of multiple polypeptide sequences. Transgenic plants comprising stacks of polynucleotide sequences can be obtained by either or both of traditional breeding methods or through genetic engineering methods. These methods include, but are not limited to, breeding individual lines each comprising a polynucleotide of interest, transforming a transgenic plant comprising a gene disclosed herein with a subsequent gene, and co-transformation of genes into a single plant cell. Co-transformation of genes can be carried out using single transformation vectors comprising multiple genes or genes carried separately on multiple vectors.

Transgenic plants comprising or derived from plant cells of this invention transformed with recombinant DNA can be further enhanced with stacked traits, e.g. a crop plant having an enhanced trait resulting from expression of DNA disclosed herein in combination with herbicide and/or pest resistance traits. For example, genes of the current invention can be stacked with other traits of agronomic interest, such as a trait providing herbicide resistance, or insect resistance, such as using a gene from Bacillus thuringensis to provide resistance against lepidopteran, coliopteran, homopteran, hemiopteran, and other insects. Herbicides for which transgenic plant tolerance has been demonstrated and the method of the present invention can be applied include, but are not limited to, glyphosate, dicamba, glufosinate, sulfonylurea, bromoxynil and norflurazon herbicides. Polynucleotide molecules encoding proteins involved in herbicide tolerance are well-known in the art and include, but are not limited to, a polynucleotide molecule encoding 5-enolpyruvylshikimate-3-phosphate synthase (EPSPS) disclosed in U.S. Pat. Nos. 5,094,945; 5,627,061; 5,633,435 and 6,040,497 for imparting glyphosate tolerance; polynucleotide molecules encoding a glyphosate oxidoreductase (GOX) disclosed in U.S. Pat. Nos. 5,463,175 and a glyphosate-N-acetyl transferase (GAT) disclosed in US Patent Application Publication 2003/0083480 A 1 also for imparting glyphosate tolerance; dicamba monooxygenase disclosed in US Patent Application Publication 2003/0135879 A 1 for imparting dicamba tolerance; a polynucleotide molecule encoding bromoxynil nitrilase (Bxn) disclosed in U.S. Pat. No. 4,810,648 for imparting bromoxynil tolerance; a polynucleotide molecule encoding phytoene desaturase (crtI) described in Misawa et al, (1993) Plant J. 4:833-840 and in Misawa et al, (1994) Plant J. 6:481-489 for norflurazon tolerance; a polynucleotide molecule encoding acetohydroxyacid synthase (AHAS, aka ALS) described in Sathasiivan et al. (1990) Nucl. Acids Res. 18:2188-2193 for imparting tolerance to sulfonylurea herbicides; polynucleotide molecules known as bar genes disclosed in DeBlock, et al. (1987) EMBO J. 6:2513-2519 for imparting glufosinate and bialaphos tolerance; polynucleotide molecules disclosed in US Patent Application Publication 2003/010609 A1 for imparting N-amino methyl phosphonic acid tolerance; polynucleotide molecules disclosed in U.S. Pat. No. 6,107,549 for imparting pyridine herbicide resistance; molecules and methods for imparting tolerance to multiple herbicides such as glyphosate, atrazine, ALS inhibitors, isoxoflutole and glufosinate herbicides are disclosed in U.S. Pat. No. 6,376,754 and US Patent Application Publication 2002/0112260. Molecules and methods for imparting insect/nematode/virus resistance are disclosed in U.S. Pat. Nos. 5,250,515; 5,880,275; 6,506,599; 5,986,175 and US Patent Application Publication 2003/0150017 A1.

### Plant Cell Transformation Methods

Numerous methods for transforming chromosomes in a plant cell nucleus with recombinant DNA are known in the art and are used in methods of preparing a transgenic plant cell nucleus cell, and plant. Two effective methods for such transformation are Agrobacterium-mediated transformation and microprojectile bombardment. Microprojectile bombardment methods are illustrated in U.S. Pat. Nos. 5,015,580 (soybean); 5,550,318 (corn); 5,538,880 (corn); 5,914,451 (soybean); 6,160,208 (corn); 6,399,861 (corn); 6,153,812 (wheat) and 6,365,807 (rice) and Agrobacterium-mediated transformation is described in U.S. Pat. Nos. 5,159,135 (cotton); 5,824,877 (soybean); 5,463,174 (canola); 5,591,616 (corn); 5,846,797 (cotton); 6,384,301 (soybean), 7,026,528 (wheat) and 6,329,571 (rice), US Patent Application Publication 2004/0087030 A 1 (cotton), and US Patent Application Publication 2001/0042257 A1 (sugar beet), all of which are incorporated herein by reference for enabling the production of transgenic plants. Transformation of plant material is practiced in tissue culture on a nutrient media, i.e. a mixture of nutrients that will allow cells to grow in vitro. Recipient cell targets include, but are not limited to, meristem cells, hypocotyls, calli, immature embryos and gametic cells such as microspores, pollen, sperm and egg cells. Callus may be initiated from tissue sources including, but not limited to, immature embryos, hypocotyls, seedling apical meristems, microspores and the like. Cells containing a transgenic nucleus are grown into transgenic plants.

In addition to direct transformation of a plant material with a recombinant DNA, a transgenic plant cell nucleus can be prepared by crossing a first plant having cells with a transgenic nucleus with recombinant DNA with a second plant lacking the transgenic nucleus. For example, recombinant DNA can be introduced into a nucleus from a first plant line that is amenable to transformation to transgenic nucleus in cells that are grown into a transgenic plant which can be crossed with a second plant line to introgress the recombinant DNA into the second plant line. A transgenic plant with recombinant DNA providing an enhanced trait, e.g. enhanced yield, can be crossed with transgenic plant line having other recombinant DNA that confers another trait, for example herbicide resistance or pest resistance, to produce progeny plants having recombinant DNA that confers both traits. Typically, in such breeding for combining traits the transgenic plant donating the additional trait is a male line and the transgenic plant carrying the base traits is the female line. The progeny of this cross will segregate such that some of the plants will carry the DNA for both parental traits and some will carry DNA for one parental trait; such plants can be identified by markers associated with parental recombinant DNA, e.g. marker identification by analysis for recombinant DNA or, in the case where a selectable marker is linked to the recombinant, by application of the selecting agent such as a herbicide for use with a herbicide tolerance marker, or by selection for the enhanced trait. Progeny plants carrying DNA for both parental traits can be crossed back into the female parent line multiple times, for example usually 6 to 8 generations, to produce a progeny plant with substantially the same genotype as one original transgenic parental line but for the recombinant DNA of the other transgenic parental line

In the practice of transformation DNA is typically introduced into only a small percentage of target plant cells in any one transformation experiment. Marker genes are used to provide an efficient system for identification of those cells that are stably transformed by receiving and integrating a recombinant DNA molecule into their genomes. Preferred marker genes provide selective markers which confer resistance to a selective agent, such as an antibiotic or a herbicide. Any of the herbicides to which plants of this invention may be resistant are useful agents for selective markers. Potentially transformed cells are exposed to the selective agent. In the population of surviving cells will be those cells where, generally, the resistance-conferring gene is integrated and expressed at sufficient levels to permit cell survival. Cells may be tested further to confirm stable integration of the exogenous DNA. Commonly used selective marker genes include those conferring resistance to antibiotics such as kanamycin and paromomycin (nptII), hygromycin B (aph IV), spectinomycin (aadA) and gentamycin (aac3 and aacC4) or resistance to herbicides such as glufosinate (bar or pat), dicamba (DMO) and glyphosate (aroA or EPSPS). Examples of such selectable markers are illustrated in U.S. Pat. Nos. 5,550,318; 5,633,435; 5,780,708 and 6,118,047. Markers which provide an ability to visually screen transformants can also be employed, for example, a gene expressing a colored or fluorescent protein such as a luciferase or green fluorescent protein (GFP) or a gene expressing a beta-glucuronidase or uidA gene (GUS) for which various chromogenic substrates are known.

Plant cells that survive exposure to the selective agent, or plant cells that have been scored positive in a screening assay, may be cultured in regeneration media and allowed to mature into plants. Developing plantlets regenerated from transformed plant cells can be transferred to plant growth mix, and hardened off, for example, in an environmentally controlled chamber at about 85% relative humidity, 600 ppm CO₂, and 25-250 microeinsteins m⁻² s⁻¹ of light, prior to transfer to a greenhouse or growth chamber for maturation. Plants are regenerated from about 6 weeks to 10 months after a transformant is identified, depending on the initial tissue, and plant species. Plants may be pollinated using conventional plant breeding methods known to those of skill in the art and seed produced, for example self-pollination is commonly used with transgenic corn. The regenerated transformed plant or its progeny seed or plants can be tested for expression of the recombinant DNA and selected for the presence of enhanced agronomic trait.

### Transgenic Plants and Seeds

Transgenic plants derived from transgenic plant cells having a transgenic nucleus of this invention are grown to generate transgenic plants having an enhanced trait as compared to a control plant and produce transgenic seed and haploid pollen of this invention. Such plants with enhanced traits are identified by selection of transformed plants or progeny seed for the enhanced trait. For efficiency a selection method is designed to evaluate multiple transgenic plants (events) comprising the recombinant DNA, for example multiple plants from 2 to 20 or more transgenic events. Transgenic plants grown from transgenic seed provided herein demonstrate improved agronomic traits that contribute to increased yield or other trait that provides increased plant value, including, for example, improved seed quality. Of particular interest are plants having enhanced water use efficiency, enhanced cold tolerance, increased yield, enhanced nitrogen use efficiency, enhanced seed protein and enhanced seed oil.

Table 1 provides a list of protein encoding DNA (“genes”) that are useful as recombinant DNA for production of transgenic plants with enhanced agronomic trait, the elements of Table 1 are described by reference to:
“PEP SEQ ID NO” identifies an amino acid sequence from SEQ ID NO: 45 to 88.
“NUC SEQ ID NO” identifies a DNA sequence from SEQ ID NO:1 to 44.
“Gene ID” refers to an arbitrary identifier.
“Gene Name” denotes a common name for the protein encoded by the recombinant DNA preceded by the abbreviated genus and species as fully defined in the sequence listing. The + or − preceding the gene name indicates whether the protein is expressed (+) or suppressed (−) in plants to provide an enhanced trait.

TABLE 1

| NUC | PEP | | |
|---|---|---|---|
| SEQ ID | SEQ ID | | |
| NO | NO | Gene ID | Gene Name |
| 1 | 45 | Mnom002981 | +Le.Etr1/NR |
| 2 | 46 | Mnom002989- | +Os.CPYC type |
| | | Mnom002990 | glutaredoxin (plastid form) |
| 3 | 47 | Mnom003067 | +Os.G1435 like 2 |
| 4 | 48 | Mnom003088 | +Ca.RAM1H1 |
| 5 | 49 | Mnom003090 | +At.cdc2 |
| 6 | 50 | Mnom003093 | +At.NADK2(NAD kinase 2) |
| 7 | 51 | Mnom003205 | +Os.Ferredoxin-NADP |
| | | | reductase, root isozyme |
| 8 | 52 | Mnom003219 | +Cc.Asparagine |
| | | | synthetase codon |
| | | | optimized |
| 9 | 53 | Mnom003220 | +At.Bidirectional |
| | | | Aminoacid Transporter 1 |
| | | | (BAT1) |
| 10 | 54 | Mnom003227 | +At.Aspartate |
| | | | aminotransferase |
| | | | Chloroplastic |
| 11 | 55 | Mnom003241 | +Os.glutathione reductase |
| | | | (GR2) like 2 sequence |
| 12 | 56 | Mnom003242 | +Os.glutathione reductase |
| | | | (GR2) like 1 |
| 13 | 57 | Mnom003243 | +At.siroheme synthase |
| 14 | 58 | Mnom003259 | +Zm.GIn1-3 |
| 15 | 59 | Mnom003266 | +At.DjA3 |
| 16 | 60 | Mnom003270 | +Zm.SLAC1 |
| 17 | 61 | Mnom003328 | +Zm.G393-2 |
| 18 | 62 | Mnom003331 | +Zm.HDZIPII-1 |
| 19 | 63 | Mnom003333 | +Zm.G398-3 |
| 20 | 64 | Mnom003444 | +Os.dep1 (Dense and |
| | | | erect panicle 1) |
| 21 | 65 | Mnom003545 | +Os.SKIPa (Ski-interacting |
| | | | protein a) |
| 22 | 66 | Mnom003601 | +At.GLB2 |
| 23 | 67 | Mnom003625 | +SI.Delta-tonoplast |
| | | | intrinsic protein |
| 24 | 68 | Mnom003228 | +At.Prokaryotic-type AAT |
| | | | Cytosolic |
| 25 | 69 | Mnom003308 | -Gr.AMP1 |
| 26 | 70 | Mnom003326 | +Zm.G395 |
| 27 | 71 | Mnom003654 | +Th.IPK2 |
| 28 | 72 | Mnom003658 | +Sr.CCaMK(Calcium |
| | | | calmodulin dependent |
| | | | protein kinase) |
| 29 | 73 | 1141368:1 | +At.G1543_NterminalSeq( |
| | | | 1.273) |
| 30 | 74 | 1124488:1 | +Zm.G2041_Truncated |
| 31 | 75 | Mnom003787, | +At.HSF2 |
| | | Mnom003792 | |
| 32 | 76 | Mnom003818 | +Pp. |
| | | | PHYPADRAFT_161210 |
| | | | Putative serine lysine |
| | | | rich |
| 33 | 77 | Mnom003819, | +Pp.PHYPADRAFT_1636 |
| | | Mnom003822 | 20 |
| 34 | 78 | Mnom003820 | +Pp. |
| | | | PHYPADRAFT_171344 |
| | | | Lys - M domain containing |
| | | | protein |
| 35 | 79 | Mnom003838 | +At.Lec2 |
| 36 | 80 | Mnom003902, | +Cg. |
| | | Mnom003907 | PHE0007661_predicted |
| | | | ornithine |
| | | | cyclodeaminase |
| 37 | 81 | Mnom003906 | +At. ChLoride Channel e |
| | | | (CICe) |
| 38 | 82 | Mnom003960 | +At.MMS21 |
| 39 | 83 | Mnom004035, | +At.CGPG838 |
| | | Mnom004036, | putative ribulose-5- |
| | | Mnom004052, | phosphate-3-epimerase |
| | | Mnom004053 | |
| 40 | 84 | Mnom004037- | +At. GAD4 (glutamate |
| | | Mnom004038, | decarboxylase4 ) |
| | | Mnom004054- | |
| | | Mnom004055 | |
| 41 | 85 | Mnom004043, | +Zm.PHE0006532_corn |
| | | Mnom004060 | 14-3-3 13 N-terminus |
| 42 | 86 | Mnom004112 | +At.KLUH |
| 43 | 87 | PHE0014906 | +Zm.PsbR |
| 44 | 88 | PHE0002227 | +Zm. protease inhibitor |
| | | | like 2 |

Selection Methods for Transgenic Plants with Enhanced Agronomic Trait

Within a population of transgenic plants each regenerated from a plant cell having a nucleus with recombinant DNA many plants that survive to fertile transgenic plants that produce seeds and progeny plants will not exhibit an enhanced agronomic trait. Selection from the population is necessary to identify one or more transgenic plant cells having a transgenic nucleus that can provide plants with the enhanced trait. Transgenic plants having enhanced traits are selected from populations of plants regenerated or derived from plant cells transformed as described herein by evaluating the plants in a variety of assays to detect an enhanced trait, e.g. enhanced water use efficiency, enhanced cold tolerance, increased yield, enhanced nitrogen use efficiency, enhanced seed protein and enhanced seed oil. These assays also may take many forms including, but not limited to, direct screening for the trait in a greenhouse or field trial or by screening for a surrogate trait. Such analyses can be directed to detecting changes in the chemical composition, biomass, physiological properties, morphology of the plant. Changes in chemical compositions such as nutritional composition of grain can be detected by analysis of the seed composition and content of protein, free amino acids, oil, free fatty acids, starch or tocopherols. Changes in biomass characteristics can be made on greenhouse or field grown plants and can include plant height, stem diameter, root and shoot dry weights; and, for corn plants, ear length and diameter. Changes in physiological properties can be identified by evaluating responses to stress conditions, for example assays using imposed stress conditions such as water deficit, nitrogen deficiency, cold growing conditions, pathogen or insect attack or light deficiency, or increased plant density. Changes in morphology can be measured by visual observation of tendency of a transformed plant with an enhanced agronomic trait to also appear to be a normal plant as compared to changes toward bushy, taller, thicker, narrower leaves, striped leaves, knotted trait, chlorosis, albino, anthocyanin production, or altered tassels, ears or roots. Other selection properties include days to pollen shed, days to silking, leaf extension rate, chlorophyll content, leaf temperature, stand, seedling vigor, internode length, plant height, leaf number, leaf area, tillering, brace roots, stay green, stalk lodging, root lodging, plant health, barreness/prolificacy, green snap, and pest resistance. In addition, phenotypic characteristics of harvested grain may be evaluated, including number of kernels per row on the ear, number of rows of kernels on the ear, kernel abortion, kernel weight, kernel size, kernel density and physical grain quality.

Assays for screening for a desired trait are readily designed by those practicing in the art. The following illustrates useful screening assays for corn traits using hybrid corn plants. The assays can be readily adapted for screening other plants such as canola, cotton and soybean either as hybrids or inbreds.

Transgenic corn plants having nitrogen use efficiency are identified by screening in fields with three levels of nitrogen (N) fertilizer being applied, e.g. low level (0 N), medium level (80 lb/ac) and high level (180 lb/ac). Plants with enhanced nitrogen use efficiency provide higher yield as compared to control plants.

Transgenic corn plants having enhanced yield are identified by screening using progeny of the transgenic plants over multiple locations with plants grown under optimal production management practices and maximum weed and pest control. A useful target for improved yield is a 5% to 10% increase in yield as compared to yield produced by plants grown from seed for a control plant. Selection methods may be applied in multiple and diverse geographic locations, for example up to 16 or more locations, over one or more planting seasons, for example at least two planting seasons, to statistically distinguish yield improvement from natural environmental effects.

Transgenic corn plants having enhanced water use efficiency are identified by screening plants in an assay where water is withheld for a period to induce stress followed by watering to revive the plants. For example, a useful selection process imposes 3 drought/re-water cycles on plants over a total period of 15 days after an initial stress free growth period of 11 days. Each cycle consists of 5 days, with no water being applied for the first four days and a water quenching on the 5th day of the cycle. The primary phenotypes analyzed by the selection method are the changes in plant growth rate as determined by height and biomass during a vegetative drought treatment.

Transgenic corn plants having enhanced cold tolerance are identified by screening plants in a cold germination assay and/or a cold tolerance field trial. In a cold germination assay trays of transgenic and control seeds are placed in a growth chamber at 9.7° C. for 24 days (no light). Seeds having higher germination rates as compared to the control are identified as having enhanced cold tolerance. In a cold tolerance field trial plants with enhanced cold tolerance are identified from field planting at an earlier date than conventional Spring planting for the field location. For example, seeds are planted into the ground around two weeks before local farmers begin to plant corn so that a significant cold stress is exerted onto the crop, named as cold treatment. Seeds also are planted under local optimal planting conditions such that the crop has little or no exposure to cold condition, named as normal treatment. At each location, seeds are planted under both cold and normal conditions preferably with multiple repetitions per treatment.

Transgenic corn plants having seeds with increased protein and/or oil levels are identified by analyzing progeny seed for protein and/or oil. Near-infrared transmittance spectrometry is a non-destructive, high-throughput method that is useful to determine the composition of a bulk seed sample for properties listed in table 2.

TABLE 2

| | |
|---|---|
| Typical sample(s): | Whole grain corn and soybean seeds |
| Typical analytical range: | Corn - moisture 5-15%, oil 5-20%, |
| | protein 5-30%, starch 50-75%, and density |
| | 1.0-1.3%. |
| | Soybean - moisture 5-15%, oil 15-25%, |
| | and protein 35-50%. |

Although the plant cells and methods of this invention can be applied to any plant cell, plant, seed or pollen, e.g. any fruit, vegetable, grass, tree or ornamental plant, the various aspects of the invention are preferably applied to corn, soybean, cotton, canola, alfalfa, wheat, rice, sugarcane, and sugar beet plants. In many cases the invention is applied to corn plants that are inherently resistant to disease from the Mal de Rio Cuarto virus or the Puccina sorghi fungus or both.

Testing for Enhanced Traits in a Model Organism

Arabidopsis thaliana is used a model for genetics and metabolism in plants. A two-step screening process was employed which included two passes of trait characterization to ensure that the trait modification was dependent on expression of the recombinant DNA, but not due to the chromosomal location of the integration of the transgene. Twelve independent transgenic lines for each recombinant DNA construct were established and assayed for the transgene expression levels. Five transgenic lines with high transgene expression levels were used in the first pass screen to evaluate the transgene's function in T2 transgenic plants. Subsequently, three transgenic events, which had been shown to have one or more enhanced traits, were further evaluated in the second pass screen to confirm the transgene's ability to impart an enhanced trait. Recombinant DNA encoding At.GLB2 (SEQ ID NO: 66) or Cg.PHE0007661_predicted ornithine cyclodeaminase (SEQ ID NO: 80) enhanced growth and development at early stages as identified by a PP screen (as further defined below) for early plant growth and development in Arabidopsis.

PP-Enhancement of Early Plant Growth and Development:

It has been known in the art that to minimize the impact of disease on crop profitability, it is important to start the season with healthy and vigorous plants. This means avoiding seed and seedling diseases, leading to increased nutrient uptake and increased yield potential. Traditionally early planting and applying fertilizer are the methods used for promoting early seedling vigor. In early development stage, plant embryos establish only the basic root-shoot axis, a cotyledon storage organ(s), and stem cell populations, called the root and shoot apical meristems that continuously generate new organs throughout post-embryonic development. “Early growth and development” used herein encompasses the stages of seed imbibition through the early vegetative phase. Plants testing positive in this assay have advantages in one or more processes including, but not limited to, germination, seedling vigor, root growth and root morphology under non-stressed conditions. The transgenic plants starting from a more robust seedling are less susceptible to the fungal and bacterial pathogens that attach germinating seeds and seedling. Furthermore, seedlings with advantage in root growth are more resistant to drought stress due to extensive and deeper root architecture. Therefore, it can be recognized by those skilled in the art that genes conferring the growth advantage in early stages to plants can also be used to generate transgenic plants that are more resistant to various stress conditions due to enhanced early plant development. As demonstrated from the model plant screen, embodiments of transgenic plants with trait-improving recombinant DNA identified in the early plant development screen can grow better under non-stress conditions and/or stress conditions providing a higher yield potential as compared to control plants.

The following examples are included to demonstrate aspects of the invention, those of skill in the art should, in light of the present disclosure, appreciate that many changes can be made in the specific aspects which are disclosed and still obtain a like or similar results without departing from the spirit and scope of the invention.

### Example 1

### Plant Expression Constructs

This example illustrates the construction of plasmids for transferring recombinant DNA into a plant cell nucleus that can be regenerated into transgenic plants.

### A. Plant Expression Constructs for Corn Transformation

A base corn transformation vector pMON93039, as set forth in SEQ ID NO:90, illustrated in Table 3, is fabricated for use in preparing recombinant DNA for Agrobacterium-mediated transformation into corn tissue.

TABLE 3

| | | | Coordinates |
|---|---|---|---|
| | | | of SEQ ID |
| Function | Name | Annotation | NO: 90 |
| Agrobacterium | B-AGRtu.right border | Agro right border sequence, | 11364-11720 |
| T-DNA transfer | | essential for transfer of T-DNA. | |
| Gene of interest | E-Os.Act1 | Upstream promoter region of the | 19-775 |
| expression | | rice actin 1 gene | |
| cassette | E-CaMV.35S.2xA1-B3 | Duplicated35S A1-B3 domain | 788-1120 |
| | | without TATA box | |
| | P-Os.Act1 | Promoter region of the rice actin 1 | 1125-1204 |
| | | gene | |
| | L-Ta.Lhcb1 | 5′ untranslated leader of wheat | 1210-1270 |
| | | major chlorophyll a/b binding | |
| | | protein | |
| | I-Os.Act1 | First intron and flanking UTR exon | 1287-1766 |
| | | sequences from the rice actin 1 | |
| | | gene | |
| | T-St.Pis4 | 3′ non-translated region of the | 1838-2780 |
| | | potato proteinase inhibitor II gene | |
| | | which functions to direct | |
| | | polyadenylation of the mRNA | |
| Plant selectable | P-Os.Act1 | Promoter from the rice actin 1 gene | 2830-3670 |
| marker | | | |
| expression | L-Os.Act1 | First exon of the rice actin 1 gene | 3671-3750 |
| cassette | | | |
| | I-Os.Act1 | First intron and flanking UTR exon | 3751-4228 |
| | | sequences from the rice actin 1 | |
| | | gene | |
| | TS-At.ShkG-CTP2 | Transit peptide region of | 4238-4465 |
| | | Arabidopsis EPSPS | |
| | CR-AGRtu.aroA- | Coding region for bacterial strain | 4466-5833 |
| | CP4.nat | CP4 native aroA gene. | |
| | T-AGRtu.nos | A 3′ non-translated region of the | 5849-6101 |
| | | nopaline synthase gene of | |
| | | Agrobacterium tumefaciens Ti | |
| | | plasmid which functions to direct | |
| | | polyadenylation of the mRNA. | |
| Agrobacterium | B-AGRtu.left border | Agro left border sequence, essential | 6168-6609 |
| T-DNA transfer | | for transfer of T-DNA. | |
| Maintenance in | OR-Ec.oriV-RK2 | The vegetative origin of replication | 6696-7092 |
| E. coli | | from plasmid RK2. | |
| | CR-Ec.rop | Coding region for repressor of | 8601-8792 |
| | | primer from the ColE1 plasmid. | |
| | | Expression of this gene product | |
| | | interferes with primer binding at the | |
| | | origin of replication, keeping | |
| | | plasmid copy number low. | |
| | OR-Ec.ori-ColE1 | The minimal origin of replication | 9220-9808 |
| | | from the E. coli plasmid ColE1. | |
| | P-Ec.aadA-SPC/STR | Promoter for Tn7 | 10339-10380 |
| | | adenylyltransferase (AAD(3″)) | |
| | CR-Ec.aadA- | Coding region for Tn7 | 10381-11169 |
| | SPC/STR | adenylyltransferase (AAD(3″)) | |
| | | conferring spectinomycin and | |
| | | streptomycin resistance. | |
| | T-Ec.aadA-SPC/STR | 3′ UTR from the Tn7 | 11170-11227 |
| | | adenylyltransferase (AAD(3″)) | |
| | | gene of E. coli. | |

To construct transformation vectors for expressing a protein identified in Table 1, primers for PCR amplification of the protein coding nucleotides are designed at or near the start and stop codons of the coding sequence, in order to eliminate most of the 5′ and 3′ untranslated regions. The protein coding nucleotides are inserted into the base vector in the gene of interest expression cassette at an insertion site, i.e. between the intron element (coordinates 1287-1766) and the polyadenylation element (coordinates 1838-2780).

To construct transformation vectors for suppressing a protein identified in Table 1, the amplified protein coding nucleotides are assembled in a sense and antisense arrangement and inserted into the base vector at the insertion site in the gene of interest expression cassette to provide transcribed RNA that will form a double-stranded RNA for RNA interference suppression of the protein. More specifically, the sense and anti-sense DNA is derived from an endogenous corn gene that expresses the corn homolog of SEQ ID NO:69.

### B. Plant Expression Constructs for Soy and Canola Transformation

Vectors for use in transformation of soybean and canola tissue are prepared having the elements of expression vector pMON82053 (SEQ ID NO: 91) as shown in Table 4 below.

TABLE 4

| | | | Coordinates |
|---|---|---|---|
| | | | of SEQ ID |
| Function | Name | Annotation | NO: 91 |
| Agrobacterium | B-AGRtu.left border | Agro left border sequence, essential | 6144-6585 |
| T-DNA transfer | | for transfer of T-DNA. | |
| Plant selectable | P-At.Act7 | Promoter from the Arabidopsis | |
| marker expression | | actin 7 gene | |
| cassette | L-At.Act7 | 5′UTR of Arabidopsis Act7 gene | |
| | I-At.Act7 | Intron from the | 6624-7861 |
| | | Arabidopsis actin gene | |
| | TS-At.ShkG-CTP2 | Transit peptide region of | 7864-8091 |
| | | Arabidopsis EPSPS | |
| | CR-AGRtu.aroA- | Synthetic CP4 coding region with | 8092-9459 |
| | CP4.nno_At | dicot preferred codon usage. | |
| | T-AGRtu.nos | A 3′ non-translated region of | 9466-9718 |
| | | the nopaline synthase gene of | |
| | | Agrobacterium tumefaciens Ti | |
| | | plasmid which functions to direct | |
| | | polyadenylation of the mRNA. | |
| Gene of interest | P-CaMV.35S-enh | Promoter for 35S RNA from CaMV | 1-613 |
| expression cassette | | containing a duplication of | |
| | | the -90 to -350 region. | |
| | T-Gb.E6-3b | 3′ untranslated region from the fiber | 688-1002 |
| | | protein E6 gene of sea-island cotton. | |
| Agrobacterium | B-AGRtu.right | Agro right border sequence, essential | 1033-1389 |
| T-DNA transfer | border | for transfer of T-DNA. | |
| Maintenance in | OR-Ec.oriV-RK2 | The vegetative origin of replication | 5661-6057 |
| E. coli | | from plasmid RK2. | |
| | CR-Ec.rop | Coding region for repressor of primer | 3961-4152 |
| | | from the ColE1 plasmid. Expression | |
| | | of this gene product interferes with | |
| | | primer binding at the origin of | |
| | | replication, keeping plasmid copy | |
| | | number low. | |
| | OR-Ec.ori-ColE1 | The minimal origin of replication | 2945-3533 |
| | | from the E. coli plasmid ColEl. | |
| | P-Ec.aadA-SPC/STR | Promoter for Tn7 | 2373-2414 |
| | | adenylyltransferase | |
| | | (AAD(3″)) | |
| | CR-Ec.aadA- | Coding region for Tn7 | 1584-2372 |
| | SPC/STR | adenylyltransferase (AAD(3″)) | |
| | | conferring spectinomycin and | |
| | | streptomycin resistance. | |
| | T-Ec.aadA-SPC/STR | 3′ UTR from the Tn7 | 1526-1583 |
| | | adenylyltransferase | |
| | | (AAD(3″)) gene of E. coli. | |

To construct transformation vectors for expressing a protein identified in Table 1, primers for PCR amplification of the protein coding nucleotides are designed at or near the start and stop codons of the coding sequence, in order to eliminate most of the 5′ and 3′ untranslated regions. The protein coding nucleotides are inserted into the base vector in the gene of interest expression cassette at an insertion site, i.e. between the promoter element (coordinates 1-613) and the polyadenylation element (coordinates 688-1002).

To construct transformation vectors for suppressing a protein identified in Table 1, the amplified protein coding nucleotides are assembled in a sense and antisense arrangement and inserted into the base vector at the insertion site in the gene of interest expression cassette to provide transcribed RNA that will form a double-stranded RNA for RNA interference suppression of the protein. More specifically, for soybean the sense and anti-sense DNA is derived from a soybean homolog of SEQ ID NO: 69, and for canola the sense and anti-sense DNA is derived from an endogenous canola gene that encodes the canola homolog of SEQ ID NO: 69.

### C. Cotton Transformation Vector

Plasmids for use in transformation of cotton tissue are prepared with elements of expression vector pMON99053 (SEQ ID NO: 92) as shown in Table 5 below.

TABLE 5

| | | | Coordinates |
|---|---|---|---|
| | | | of SEQ ID |
| Function | Name | Annotation | NO: 92 |
| Agrobacterium | B-AGRtu.right border | Agro right border sequence, | 1-357 |
| T-DNA transfer | | essential for transfer of T-DNA. | |
| Gene of interest | Exp-CaMV.35S- | Enhanced version of the 35S RNA | 388-1091 |
| expression | enh+Ph.DnaK | promoter from CaMV plus the | |
| cassette | | petunia hsp70 5′ untranslated region | |
| | T-Ps.RbcS2-E9 | The 3' non-translated region of the | 1165-1797 |
| | | pea RbcS2 gene which functions to | |
| | | direct polyadenylation of the | |
| | | mRNA. | |
| Plant selectable | Exp-CaMV.35S | Promoter and 5′ untranslated region | 1828-2151 |
| marker | | from the 35S RNA of CaMV | |
| expression | CR-Ec.nptII-Tn5 | Coding region for neomycin | 2185-2979 |
| cassette | | phosphotransferase gene from | |
| | | transposon Tn5 which confers | |
| | | resistance to neomycin and | |
| | | kanamycin. | |
| | T-AGRtu.nos | A 3′ non-translated region of the | 3011-3263 |
| | | nopaline synthase gene of | |
| | | Agrobacterium tumefaciens Ti | |
| | | plasmid which functions to direct | |
| | | polyadenylation of the mRNA. | |
| Agrobacterium | B-AGRtu.left border | Agro left border sequence, | 3309-3750 |
| T-DNA transfer | | essential for transfer of T-DNA. | |
| Maintenance in | OR-Ec.oriV-RK2 | The vegetative origin of replication | 3837-4233 |
| E. coli | | from plasmid RK2. | |
| | CR-Ec.rop | Coding region for repressor of | 5742-5933 |
| | | primer from the ColE1 plasmid. | |
| | | Expression of this gene product | |
| | | interferes with primer binding at | |
| | | theorigin of replication, keeping | |
| | | plasmid copy number low. | |
| | OR-Ec.ori-ColE1 | The minimal origin of replication | 6361-6949 |
| | | from the E. coli plasmid ColE1. | |
| | P-Ec.aadA-SPC/STR | Promoter for Tn7 | 7480-7521 |
| | | adenylyltransferase (AAD(3″)) | |
| | CR-Ec.aadA-SPC/STR | Coding region for Tn7 | 7522-8310 |
| | | adenylyltransferase (AAD(3″)) | |
| | | conferring spectinomycin and | |
| | | streptomycin resistance. | |
| | T-Ec.aadA-SPC/STR | 3′ UTR from the Tn7 | 8311-8368 |
| | | adenylyltransferase (AAD(3″)) | |
| | | gene of E. coli. | |

To construct transformation vectors for expressing a protein identified in Table 1, primers for PCR amplification of the protein coding nucleotides are designed at or near the start and stop codons of the coding sequence, in order to eliminate most of the 5′ and 3′ untranslated regions. The protein coding nucleotides are inserted into the base vector in the gene of interest expression cassette at an insertion site, i.e. between the promoter element (coordinates 388-1091) and the polyadenylation element (coordinates 1165-1797).

To construct transformation vectors for suppressing a protein identified in Table 1, the amplified protein coding nucleotides are assembled in a sense and antisense arrangement and inserted into the base vector at the insertion site in the gene of interest expression cassette to provide transcribed RNA that will form a double-stranded RNA for RNA interference suppression of the protein. More specifically, the sense and anti-sense DNA is derived from an endogenous cotton gene that encodes SEQ ID NO: 69.

### Example 2

### Corn Transformation

This example illustrates transformation methods useful in producing a transgenic nucleus in a corn plant cell, and the plants, seeds and pollen produced from a transgenic cell with such a nucleus having an enhanced trait, i.e. enhanced water use efficiency, enhanced cold tolerance, increased yield, enhanced nitrogen use efficiency, enhanced seed protein and enhanced seed oil. A plasmid vector is prepared by cloning the DNA of SEQ ID NO:1 into the gene of interest expression cassette in the base vector for use in corn transformation of corn tissue provided in Example 1, Table 3.

For Agrobacterium-mediated transformation of corn embryo cells corn plants of a readily transformable line are grown in the greenhouse and ears are harvested when the embryos are 1.5 to 2.0 mm in length. Ears are surface sterilized by spraying or soaking the ears in 80% ethanol, followed by air drying. Immature embryos are isolated from individual kernels on surface sterilized ears. Prior to inoculation of maize cells, Agrobacterium cells are grown overnight at room temperature. Immature maize embryo cells are inoculated with Agrobacterium shortly after excision, and incubated at room temperature with Agrobacterium for 5-20 minutes. Immature embryo plant cells are then co-cultured with Agrobacterium for 1 to 3 days at 23° C. in the dark. Co-cultured embryos are transferred to selection media and cultured for approximately two weeks to allow embryogenic callus to develop. Embryogenic callus is transferred to culture medium containing 100 mg/L paromomycin and subcultured at about two week intervals. Transformed plant cells are recovered 6 to 8 weeks after initiation of selection.

For Agrobacterium-mediated transformation of maize callus immature embryos are cultured for approximately 8-21 days after excision to allow callus to develop. Callus is then incubated for about 30 minutes at room temperature with the Agrobacterium suspension, followed by removal of the liquid by aspiration. The callus and Agrobacterium are co-cultured without selection for 3-6 days followed by selection on paromomycin for approximately 6 weeks, with biweekly transfers to fresh media. Paromomycin resistant calli are identified about 6-8 weeks after initiation of selection.

To regenerate transgenic corn plants a callus of transgenic plant cells resulting from transformation and selection is placed on media to initiate shoot development into plantlets which are transferred to potting soil for initial growth in a growth chamber at 26° C. followed by a mist bench before transplanting to 5 inch pots where plants are grown to maturity. The regenerated plants are self-fertilized and seed is harvested for use in one or more methods to select seeds, seedlings or progeny second generation transgenic plants (R2 plants) or hybrids, e.g. by selecting transgenic plants exhibiting an enhanced trait as compared to a control plant.

The above process is repeated to produce multiple events of transgenic corn plant cells that are transformed with recombinant DNA from each of the genes identified in Table 1. Events are designed to produce in the transgenic cells one of the proteins identified in Table 1, except the corn homolog of SEQ ID NO: 69, which is suppressed. Progeny transgenic plants and seed of the transformed plant cells are screened for enhanced water use efficiency, enhanced cold tolerance, increased yield, enhanced nitrogen use efficiency, enhanced seed protein and enhanced seed oil. From each group of multiple events of transgenic plants with a specific recombinant DNA from Table 1 the event that produces the greatest enhancement in yield, water use efficiency, nitrogen use efficiency, enhanced cold tolerance, enhanced seed protein and enhanced seed oil is identified and progeny seed is selected for commercial development.

### Example 3

### Soybean Transformation

This example illustrates plant transformation useful in producing a transgenic nucleus in a soybean plant cell, and the plants, seeds and pollen produced from a transgenic cell with such a nucleus having an enhanced trait, i.e. enhanced water use efficiency, enhanced cold tolerance, increased yield, enhanced nitrogen use efficiency, enhanced seed protein and enhanced seed oil.

For Agrobacterium mediated transformation, soybean seeds are imbided overnight and the meristem explants excised. The explants are placed in a wounding vessel. Soybean explants and induced Agrobacterium cells from a strain containing plasmid DNA with the gene of interest cassette and a plant selectable marker cassette are mixed no later than 14 hours from the time of initiation of seed imbibition, and wounded using sonication. Following wounding, explants are placed in co-culture for 2-5 days at which point they are transferred to selection media for 6-8 weeks to allow selection and growth of transgenic shoots. Resistant shoots are harvested approximately 6-8 weeks and placed into selective rooting media for 2-3 weeks. Shoots producing roots are transferred to the greenhouse and potted in soil. Shoots that remain healthy on selection, but do not produce roots are transferred to non-selective rooting media for an additional two weeks. Roots from any shoots that produce roots off selection are tested for expression of the plant selectable marker before they are transferred to the greenhouse and potted in soil.

The above process is repeated to produce multiple events of transgenic soybean plant cells that are transformed with recombinant DNA from each of the genes identified in Table 1. Events are designed to produce in the transgenic cells one of the proteins identified in Table 1, except the soybean homolog of SEQ ID NOs: 69, which is suppressed. Progeny transgenic plants and seed of the transformed plant cells are screened for enhanced water use efficiency, enhanced cold tolerance, increased yield, enhanced seed protein and enhanced seed oil. From each group of multiple events of transgenic plants with a specific recombinant DNA from Table 1 the event that produces the greatest enhancement in yield, water use efficiency, nitrogen use efficiency, enhanced cold tolerance, enhanced seed protein and enhanced seed oil is identified and progeny seed is selected for commercial development.

### Example 4

### Cotton Transgenic Plants with Enhanced Agronomic Traits

This example illustrates plant transformation useful in producing a transgenic nucleus in a cotton plant cell, and the plants, seeds and pollen produced from a transgenic cell with such a nucleus having an enhanced trait, i.e. enhanced water use efficiency, increased yield, enhanced nitrogen use efficiency and enhanced seed oil.

Transgenic cotton plants containing each recombinant DNA having a sequence of SEQ ID NO: 1 through SEQ ID NO: 44 are obtained by transforming with recombinant DNA from each of the genes identified in Table 1 using Agrobacterium-mediated transformation. The above process is repeated to produce multiple events of transgenic cotton plant cells that are transformed with recombinant DNA from each of the genes identified in Table 1. Events are designed to produce in the transgenic cells one of the proteins identified in Table 1, except the cotton gene encoding the protein of SEQ ID NO: 69, which is suppressed.

From each group of multiple events of transgenic plants with a specific recombinant DNA from Table 1 the event that produces the greatest enhancement in yield, water use efficiency, nitrogen use efficiency, enhanced cold tolerance, enhanced seed protein and enhanced seed oil is identified and progeny seed is selected for commercial development.

Progeny transgenic plants are selected from a population of transgenic cotton events under specified growing conditions and are compared with control cotton plants. Control cotton plants are substantially the same cotton genotype but without the recombinant DNA, for example, either a parental cotton plant of the same genotype that was not transformed with the identical recombinant DNA or a negative isoline of the transformed plant. Additionally, a commercial cotton cultivar adapted to the geographical region and cultivation conditions, i.e. cotton variety ST474, cotton variety FM 958, and cotton variety Siokra L-23, are used to compare the relative performance of the transgenic cotton plants containing the recombinant DNA.

Transgenic cotton plants with enhanced yield and water use efficiency are identified by growing under variable water conditions. Specific conditions for cotton include growing a first set of transgenic and control plants under “wet” conditions, i.e. irrigated in the range of 85 to 100 percent of evapotranspiration to provide leaf water potential of −14 to −18 bars, and growing a second set of transgenic and control plants under “dry” conditions, i.e. irrigated in the range of 40 to 60 percent of evapotranspiration to provide a leaf water potential of −21 to −25 bars. Pest control, such as weed and insect control is applied equally to both wet and dry treatments as needed. Data gathered during the trial includes weather records throughout the growing season including detailed records of rainfall; soil characterization information; any herbicide or insecticide applications; any gross agronomic differences observed such as leaf morphology, branching habit, leaf color, time to flowering, and fruiting pattern; plant height at various points during the trial; stand density; node and fruit number including node above white flower and node above crack boll measurements; and visual wilt scoring. Cotton boll samples are taken and analyzed for lint fraction and fiber quality. The cotton is harvested at the normal harvest timeframe for the trial area. Enhanced water use efficiency is indicated by increased yield, improved relative water content, enhanced leaf water potential, increased biomass, enhanced leaf extension rates, and improved fiber parameters.

### Example 5

### Canola Transformation

This example illustrates plant transformation useful in producing the transgenic canola plants of this invention and the production and identification of transgenic seed for transgenic canola having enhanced water use efficiency, enhanced cold tolerance, increased yield, enhanced nitrogen use efficiency, enhanced seed protein and enhanced seed oil.

Tissues from in vitro grown canola seedlings are prepared and inoculated with overnight-grown Agrobacterium cells containing plasmid DNA with the gene of interest cassette and a plant selectable marker cassette. Following co-cultivation with Agrobacterium, the infected tissues are allowed to grow on selection to promote growth of transgenic shoots, followed by growth of roots from the transgenic shoots. The selected plantlets are then transferred to the greenhouse and potted in soil. Molecular characterizations are performed to confirm the presence of the gene of interest, and its expression in transgenic plants and progenies. Progeny transgenic plants are selected from a population of transgenic canola events under specified growing conditions and are compared with control canola plants. Control canola plants are substantially the same canola genotype but without the recombinant DNA, for example, either a parental canola plant of the same genotype that is not transformed with the identical recombinant DNA or a negative isoline of the transformed plant.

Transgenic canola plant cells are transformed with each of the recombinant DNA identified in Table 1. The above process is repeated to produce multiple events of transgenic canola plant cells that are transformed with recombinant DNA from each of the genes identified in Table 1. Events are designed to produce in the transgenic cells one of the proteins identified in Table 1, except the canola homolog of the protein of SEQ ID NO: 69, which is suppressed. Progeny transgenic plants and seed of the transformed plant cells are screened for enhanced water use efficiency, enhanced cold tolerance, increased yield, enhanced seed protein and enhanced seed oil. From each group of multiple events of transgenic plants with a specific recombinant DNA from Table 1 the event that produces the greatest enhancement in yield, water use efficiency, nitrogen use efficiency, enhanced cold tolerance, enhanced seed protein and enhanced seed oil is identified and progeny seed is selected for commercial development.

### Example 6

### Homolog Identification

This example illustrates the identification of homologs of proteins encoded by the DNA identified in Table 1 which is used to provide transgenic seed and plants having enhanced agronomic traits. From the sequence of the homologs, homologous DNA sequence can be identified for preparing additional transgenic seeds and plants of this invention with enhanced agronomic traits.

An “All Protein Database” is constructed of known protein sequences using a proprietary sequence database and the National Center for Biotechnology Information (NCBI) non-redundant amino acid database (nr.aa). For each organism from which a polynucleotide sequence provided herein is obtained, an “Organism Protein Database” is constructed of known protein sequences of the organism; it is a subset of the All Protein Database based on the NCBI taxonomy ID for the organism.

The All Protein Database is queried using amino acid sequences provided herein as SEQ ID NO: 45 through SEQ ID NO: 88 using NCBI “blastp” program with E-value cutoff of 1e-8. Up to 1000 top hits are kept, and separated by organism names. For each organism other than that of the query sequence, a list is kept for hits from the query organism itself with a more significant E-value than the best hit of the organism. The list contains likely duplicated genes of the polynucleotides provided herein, and is referred to as the Core List. Another list is kept for all the hits from each organism, sorted by E-value, and referred to as the Hit List.

The Organism Protein Database is queried using polypeptide sequences provided herein as SEQ ID NO: 45 through SEQ ID NO: 88 using NCBI “blastp” program with E-value cutoff of 1e-4. Up to 1000 top hits are kept. A BLAST searchable database is constructed based on these hits, and is referred to as “SubDB”. SubDB is queried with each sequence in the Hit List using NCBI “blastp” program with E-value cutoff of 1e-8. The hit with the best E-value is compared with the Core List from the corresponding organism. The hit is deemed a likely ortholog if it belongs to the Core List, otherwise it is deemed not a likely ortholog and there is no further search of sequences in the Hit List for the same organism.

Recombinant DNA constructs are prepared using the DNA encoding each of the identified homologs and the constructs are used to prepare multiple events of transgenic corn, soybean, canola and cotton plants as illustrated in Examples 2-5. Plants are regenerated from the transformed plant cells and used to produce progeny plants and seed that are screened for enhanced water use efficiency, enhanced cold tolerance, increased yield, enhanced nitrogen use efficiency, enhanced seed protein and enhanced seed oil. From each group of multiple events of transgenic plants with a specific recombinant DNA for a homolog the event that produces the greatest enhancement in yield, water use efficiency, nitrogen use efficiency, enhanced cold tolerance, enhanced seed protein and enhanced seed oil is identified and progeny seed is selected for commercial development.

### Example 7

### Testing in Arabidopsis

A. Plant Expression Constructs for Arabidopsis Transformation

The genes encoding At.GLB2 (SEQ ID NO: 66) and Cg.PHE0007661_predicted ornithine cyclodeaminase (SEQ ID NO: 80) were amplified using primers specific to sequences upstream and downstream of the coding region. Transformation vectors were prepared to constitutively transcribe each DNA in sense orientation (for enhanced protein expression) under the control of an enhanced Cauliflower Mosaic Virus 35S promoter (U.S. Pat. No. 5,359,142). The transformation vectors also contained a bar gene as a selectable marker for resistance to glufosinate herbicide. The transformation of Arabidopsis plants was carried out using the vacuum infiltration method known in the art (Bethtold, e.g., Methods Mol. Biol. 82:259-66, 1998). Seeds harvested from the plants, named as T1 seeds, were subsequently grown in a glufosinate-containing selective medium to select for plants which were actually transformed and which produced T2 transgenic seed.

B. Early Plant Growth and Development (PP) Screen

A plate based phenotypic analysis platform was used for the rapid detection of phenotypes that are evident during the first two weeks of growth. This screen demonstrated the ability of At.GLB2 (SEQ ID NO:66) or Cg.PHE0007661_predicted ornithine cyclodeaminase (SEQ ID NO: 80) to confer advantages in the processes of germination, seedling vigor, root growth and root morphology under non-stressed growth conditions to plants. The transgenic plants with advantages in seedling growth and development were determined by the seedling weight and root length at day14 after seed planting.

T2 seeds were plated on glufosinate selection plates and grown under standard conditions (˜100 uE/m²/s, 16 h photoperiod, 22° C. at day, 20° C. at night). Seeds were stratified for 3 days at 4° C. Seedlings were grown vertically (at a temperature of 22° C. at day 20° C. at night). Observations were taken on day 10 and day 14. Both seedling weight and root length at day 14 were analyzed as quantitative responses according to example 1M.

As shown in table 6, transgenic Arabidopsis plants expressing At.GLB2 (SEQ ID NO: 66) demonstrated a significant increase in root length and transgenic Arabidopsis plants expressing Cg.PHE0007661_predicted ornithine cyclodeaminase (SEQ ID NO: 80) demonstrated a significant increase in seedling weight.

TABLE 6

| | | | Root | | Root | | Seedling | |
|---|---|---|---|---|---|---|---|---|
| | | | length at | | length at | | weight | |
| | | | day 10 | | day 14 | | at day 14 | |
| Nuc | PEP | Construct | Delta | | Delta | | Delta | |
| SEQ ID | SEQ ID | ID | mean | P-value | mean | P-value | mean | P-value |
| 22 | 66 | 80372 | 0.2015 | 0.0561 | 0.2544 | 0.0187 | 0.1617 | 0.2996 |
| 36 | 80 | 80267 | | | 0.1270 | 0.4899 | 0.5905 | 0.0373 |

C. Statistical Analyses

The measurements (M) of each plant were transformed by log₂ calculation. The Delta was calculated as log₂M(transgenic)-log₂M(reference). Two criteria were used to determine trait enhancement. The measurements (M) of each plant were transformed by log₂ calculation. The Delta was calculated as log₂M(transgenic)-log₂M (reference).

For the first criteria, the Deltas from multiple events expressing At.GLB2 were evaluated for statistical significance by t-test using SAS statistical software (SAS 9, SAS/STAT User's Guide, SAS Institute Inc, Cary, N.C., USA). A delta with a value greater than 0 indicates that the transgenic plants perform better than the reference. A delta with a value less than 0 indicates that the transgenic plants perform worse than the reference. The Delta with a value equal to 0 indicates that the performance of the transgenic plants and the reference don't show any difference. If p<0.05 and risk score mean >0, the transgenic plants showed statistically significant trait enhancement as compared to the reference. If p<0.2 and risk score mean >0, the transgenic plants showed a trend of trait enhancement as compared to the reference.

For the second criteria, the delta from each event was evaluated for statistical significance by t-test using SAS statistical software (SAS 9, SAS/STAT User's Guide, SAS Institute Inc., Cary, N.C., USA). The Delta with a value greater than 0 indicates that the transgenic plants from this event performs better than the reference. The Delta with a value less than 0 indicates that the transgenic plants from this event perform worse than the reference. The Delta with a value equal to 0 indicates that the performance of the transgenic plants from this event and the reference don't show any difference. If p<0.05 and delta mean >0, the transgenic plants from this event showed statistically significant trait improvement as compared to the reference. If p<0.2 and delta mean >0, the transgenic plants showed a trend of trait enhancement as compared to the reference. If two or more events of the transgene of interest showed enhancement in the same response, the transgene was deemed to show trait improvement.

What is claimed is:

1. A recombinant DNA construct comprising a promoter that is functional in a plant cell and that is operably linked to a polynucleotide that, when expressed in a plant cell:
(a) encodes a protein:
i) having an amino acid sequence selected from the group consisting of SEQ ID NO: 45-68, and 70-88;
ii) having an amino acid sequence having at least 90% identity over at least 90% of a reference sequence selected from the group consisting of 45-68, and 70-88 when said amino acid sequence is aligned to said reference sequence; or
iii) that is a homolog of a protein with an amino acid sequence selected from the group consisting of SEQ ID NO: 45-68, and 70-88;
or
(b) is transcribed into an RNA molecule that suppresses the level of an endogenous protein in said plant cell wherein said endogenous protein has an amino acid sequence of SEQ ID NO: 69 or is a homolog thereof;
wherein said construct is stably integrated into plant chromosomal DNA.

2. A transgenic plant cell comprising the recombinant DNA construct of claim 1 wherein said plant cell is in a plant selected by screening a population of transgenic plants that have been transformed with said construct for an enhanced trait as compared to control plants; and wherein said enhanced trait is enhanced water use efficiency, enhanced cold tolerance, increased yield, enhanced nitrogen use efficiency, enhanced seed protein or enhanced seed oil.

3. The plant cell of claim 2 further comprising DNA expressing a protein that provides tolerance from exposure to an herbicide comprising an agent applied at levels that are lethal to a wild type of said plant cell.

4. The plant cell of claim 3 wherein the agent of said herbicide is a glyphosate, dicamba, or glufosinate compound.

5. A transgenic plant comprising a plurality of plant cells of claim 2.

6. The transgenic plant of claim 5 which is homozygous for said recombinant DNA.

7. A transgenic seed comprising a plurality of plant cells of claim 2.

8. The transgenic seed of claim 7 from a corn, soybean, cotton, canola, alfalfa, wheat, rice, sugarcane, or sugar beet plant.

9. Grain comprising transgenic seed identifiable by the recombinant DNA construct of claim 1.

10. Seed meal produced from transgenic seed identifiable by the recombinant DNA construct of claim 1.

11. A transgenic pollen grain comprising a haploid derivative of a plant cell nucleus having a chromosome comprising the recombinant DNA construct of claim 1.

12. A method for manufacturing non-natural, transgenic seed that can be used to produced crop of transgenic plants with an enhanced trait resulting from expression of the stably-integrated, recombinant DNA construct of claim 1, said method comprising:
(a) screening a population of plants for said enhanced trait and said recombinant DNA, wherein individual plants in said population exhibit said trait at a level less than, essentially the same as or greater than the level that said trait is exhibited in control plants which do not contain said recombinant DNA, wherein said enhanced trait is selected from the group of enhanced traits consisting of enhanced water use efficiency, enhanced cold tolerance, increased yield, enhanced nitrogen use efficiency, enhanced seed protein and enhanced seed oil;
(b) selecting from said population one or more plants that exhibit said trait at a level greater than the level that said trait is exhibited in control plants, and
(c) collecting seed from selected plant from step b.

13. The method of claim 12 wherein said method for manufacturing said transgenic seed further comprises:
(a) verifying that said recombinant DNA is stably integrated in said selected plants, and
(b) analyzing tissue of said selected plant to determine the expression or suppression of a protein having the function of a protein having an amino acid sequence selected from the group consisting of one of SEQ ID NOs: 45-88.

14. The method of claim 13 wherein said seed is corn, soybean, cotton, canola, alfalfa, wheat, rice, sugarcane, or sugar beet seed.

15. A method of producing hybrid corn seed comprising:
(a) acquiring hybrid corn seed from an herbicide tolerant corn plant which also has the stably-integrated, recombinant DNA construct of claim 1;
(b) producing corn plants from said hybrid corn seed, wherein a fraction of the plants produced from said hybrid corn seed is homozygous for said recombinant DNA, a fraction of the plants produced from said hybrid corn seed is hemizygous for said recombinant DNA, and a fraction of the plants produced from said hybrid corn seed has none of said recombinant DNA;
(c) selecting corn plants which are homozygous and hemizygous for said recombinant DNA by treating with an herbicide;
(d) collecting seed from herbicide-treated-surviving corn plants and planting said seed to produce further progeny corn plants;
(e) repeating steps (c) and (d) at least once to produce an inbred corn line; and
(f) crossing said inbred corn line with a second corn line to produce hybrid seed.
